(19) United States
(12) Patent Application Publication
(10) Pub. No.: US 2006/0135483 A1
(43) Pub. Date: Jun. 22, 2006

(54) Oxygen/nitrogen heterocycle inhibitors of tyrosine phosphatases

(76) Inventors: Zacharia S. Cheruvallath, San Diego, CA (US); Joseph E. Semple, San Diego, CA (US); Jing Wang, San Diego, CA (US); Ruth F. Nutt, Santa Fe, CA (US); Shankari Mylvaganam, San Diego, CA (US)

Correspondence Address: JONES DAY, 222 EAST 41ST ST, NEW YORK, NY, 10017, US

(21) Appl. No.: 11/177,539

(22) Filed: Jul. 8, 2005

Related U.S. Application Data
(60) Provisional application No. 60/587,023, filed on Jul. 9, 2004.
(60) Provisional application No. 60/634,450, filed on Dec. 8, 2004.
(60) Provisional application No. 60/638,563, filed on Dec. 22, 2004.

(51) Int. Cl. A61K 31/675; C07F 9/653

(52) U.S. Cl. 514/92; 548/112

(57) ABSTRACT

Compounds, pharmaceutical compositions, and methods for treating, preventing, or ameliorating symptoms associated with diseases such as diabetes, cancer, neurodegenerative diseases, and obesity are provided. The compounds and compositions inhibit protein tyrosine phosphatase enzymes, e.g., PTP-1B.

### RELATED APPLICATIONS

Priority is claimed herein under 35 U.S.C. §119(e) to U.S. provisional patent application Nos. 60/587,023, filed Jul. 9, 2004; 60/634,450, filed Dec. 8, 2004; and 60/638,563, filed Dec. 22, 2004. The disclosures of the above-referenced applications are incorporated herein by reference in their entirety.

### TECHNICAL FIELD

Provided herein are methods of inhibiting the activity of tyrosine phosphatases that regulate signal transduction, and, more particularly, use of Oxygen/Nitrogen Heterocycle compounds and compositions as tyrosine phosphatase inhibitors for the treatment of conditions and diseases that respond to phosphatase inhibition.

### BACKGROUND

Cellular signal transduction is a fundamental mechanism whereby external stimuli that regulate cellular processes are relayed to the interior of cells. The biochemical pathways through which signals are transmitted within cells comprise a circuitry of directly or functionally connected interactive proteins. One of the key biochemical mechanisms of signal transduction involves the reversible phosphorylation of tyrosine residues on proteins. The phosphorylation state of a protein may affect its conformation and/or enzymatic activity as well as its cellular location. The phosphorylation state of a protein is modified through the reciprocal actions of protein tyrosine kinases (PTKs) and protein tyrosine phosphatases (PTPs) at various specific tyrosine residues.

A common mechanism by which receptors regulate cell function is through an inducible tyrosine kinase activity which is either endogenous to the receptor or is imparted by other proteins that become associated with the receptor (Darnell et al., 1994, Science 264:1415-1421; Heldin, 1995, Cell 80:213-223; Pawson, 1995, Nature 373:573-580).

Protein tyrosine kinases comprise a large family of transmembrane receptor and intracellular enzymes with multiple functional domains (Taylor et al., 1992 Ann. Rev. Cell Biol. 8:429-62). The binding of ligand allosterically transduces a signal across the cell membrane where the cytoplasmic portion of the PTKs initiates a cascade of molecular interactions that disseminate the signal throughout the cell and into the nucleus. Many receptor protein tyrosine kinase (RPTKs), such as epidermal growth factor receptor (EGFR) and platelet-derived growth factor receptor (PDGFR) undergo oligomerization upon ligand binding, and the receptors self-phosphorylate (via autophosphorylation or transphosphorylation) on specific tyrosine residues in the cytoplasmic portions of the receptor (Schlessinger and Ullrich, 1992, Neuron, 9:383-91, Heldin, 1995, Cell 80:213-223). Cytoplasmic protein tyrosine kinases (CPTKs), such as Janus kinases (e.g., JAK1, JAK2, TYK2) and Src kinases (e.g., src, lck, fyn), are associated with receptors for cytokines (e.g., IL-2, IL-3, IL-6, erythropoietin) and interferons, and antigen receptors. These receptors also undergo oligomerization and have tyrosine residues that become phosphorylated during activation, but the receptor polypeptides themselves do not possess kinase activity.

Like the PTKs, the protein tyrosine phosphatases (PTPs) comprise a family of transmembrane and cytoplasmic enzymes, possessing at least an approximately 230 amino acid catalytic domain containing a highly conserved active site with the consensus motif >I/V!HCXAGXXR>S/T!G. The substrates of PTPs may be PTKs which possess phosphotyrosine residues or the substrates of PTKs (Hunter, 1989, Cell 58:1013-16; Fischer et al., 1991, Science 253:401-6; Saito & Streuli, 1991, Cell Growth and Differentiation 2:59-65; Pot and Dixon, 1992, Biochem. Biophys. Acta 1136:35-43). Among these, Protein Tyrosine Phosphatase-1B (PTP-1B) is an intracellular protein found in various human tissues (Charbonneau et al., 1989, Proc. Natl. Acad. Sci. USA 86:5252-5256; Goldstein, 1993, Receptor 3:1-15).

Transmembrane or receptor-like PTPs (RPTPs) possess an extracellular domain, a single transmembrane domain, and one or two catalytic domains followed by a short cytoplasmic tail. The extracellular domains of these RPTPs are highly divergent, with small glycosylated segments (e.g., RPTPα, RPTPε), tandem repeats of immunoglobulin-like and/or fibronectin type III domains (e.g., LAR) or carbonic anhydrase like domains (e.g., RPTPγ, RPTPβ). These extracellular features might suggest that these RPTPs function as a receptor on the cell surface, and their enzymatic activity might be modulated by ligands. Intracellular or cytoplasmic PTPs (CPTPs), such as PTP1C, PTP1D, typically contain a single catalytic domain flanked by several types of modular conserved domains. For example, PTP1C, a hemopoietic cell CPTP is characterized by two Src-homology homology 2 (SH2) domains that recognize short peptide motifs bearing phosphotyrosine (pTyr).

In general, these modular conserved domains influence the intracellular localization of the protein. SH2-containing proteins are able to bind pTyr sites in activated receptors and cytoplasmic phosphoproteins. Another conserved domain known as SH3 binds to proteins with proline-rich regions. A third type known as pleckstrin-homology (PH) domain has also been identified. These modular domains have been found in both CPTKs and CPTPs as well as in non-catalytic adapter molecules, such as Grbs (Growth factor Receptor Bound), which mediate protein-protein interactions between components of the signal transduction pathway (Skolnik et al., 1991, Cell 65:83-90; Pawson, 1995, Nature 373:573-580).

Multiprotein signaling complexes comprising receptor subunits, kinases, phosphatases and adapter molecules are assembled in subcellular compartments through the specific and dynamic interactions between these domains with their binding motifs. Such signaling complexes integrate the extracellular signal from the ligand-bound receptor and relay the signal to other downstream signaling proteins or complexes in other locations inside the cell or in the nucleus (Koch et al., 1991, Science 252:668-674; Pawson, 1994, Nature 373:573-580; Mauro et al., 1994, Trends Biochem Sci 19:151-155; Cohen et al., 1995, Cell 80:237-248).

The levels of tyrosine phosphorylation required for normal cell growth and differentiation at any time are achieved through the coordinated action of PTKs and PTPS. Depending on the cellular context, these two types of enzymes may either antagonize or cooperate with each other during signal transduction. An imbalance between these enzymes may impair normal cell functions leading to metabolic disorders and cellular transformation.

For example, insulin binding to the insulin receptor, which is a PTK, triggers a variety of metabolic and growth promoting effects such as glucose transport, biosynthesis of glycogen and fats, DNA synthesis, cell division and differentiation. Diabetes mellitus, which is characterized by insufficient or a lack of insulin signal transduction, can be caused by any abnormality at any step along the insulin signaling pathway (Olefsky, 1988, in “Cecil Textbook of Medicine,” 18th Ed., 2:1360-81).

It is also well known, for example, that the overexpression of PTKs, such as HER2, can play a decisive role in the development of cancer (Slamon et al., 1987, Science 235:77-82) and that antibodies capable of blocking the activity of this enzyme can abrogate tumor growth (Drebin et al., 1988, Oncogene 2:387-394). Blocking the signal transduction capability of tyrosine kinases such as Flk-1 and the PDGF receptor have been shown to block tumor growth in animal models (Millauer et al., 1994, Nature 367:577; Ueno et al., Science 252:844-848).

Tyrosine phosphatases also play a role in signal transduction. For example, ectopic expression of RPTPα produces a transformed phenotype in embryonic fibroblasts (Zheng et al., Nature 359:336-339), and overexpression of RPTPα in embryonal carcinoma cells causes the cells to differentiate into a cell type with neuronal phenotype (den Hertog et al., EMBO J 12:3789-3798). The gene for human RPTPγ has been localized to chromosome 3p21 which is a segment frequently altered in renal and small lung carcinoma. Mutations may occur in the extracellular segment of RPTPγ, which result in RPTPs that no longer respond to external signals (LaForgia et al., Wary et al., 1993, Cancer Res 52:478-482). Mutations in the gene encoding PTP1C (also known as HCP, SHP) are the cause of the motheaten phenotype in mice which suffer severe immunodeficiency, and systemic autoimmune disease accompanied by hyperproliferation of macrophages (Schultz et al., 1993, Cell 73:1445-1454). PTP1D (also known as Syp or PTP2C) has been shown to bind through SH2 domains to sites of phosphorylation in PDGFR, EGFR and insulin receptor substrate 1 (IRS-1). Reducing the activity of PTP1D by microinjection of anti-PTPID antibody has been shown to block insulin or EGF-induced mitogenesis (Xiao et al., 1994, J Biol Chem 269:21244-21248).

Much effort has been devoted to determining which proteins are substrates of PTP-1B. One such identified substrate is the insulin receptor. The binding of insulin to its receptor results in autophosphorylation of the receptor, most notably on tyrosines 1146, 1150, and 1151 in the kinase catalytic domain (White & Kahn, 1994, J. Biol. Chem. 269:1-4). This activates the insulin receptor tyrosine kinase, and phosphorylates the insulin receptor substrate proteins that propagate the insulin-signaling event to mediate insulin's various biological effects.

A glutathione S-transferase (GST) fusion protein of PTP-1B that had a point mutation in the PTP-1B catalytic domain was constructed by Seely et al., 1996, Diabetes 45:1379-1385. Although catalytically inactive, this fusion protein was able to bind to the insulin receptor, as demonstrated by its ability to precipitate the insulin receptor from purified receptor preparations and from whole cell lysates derived from cells expressing the insulin receptor.

Recently, it was reported that PTP-1B is a negative regulator of the insulin signalling pathway (Kennedy et al., 1999, Science 283:1544-1548). It is also known that mice lacking PTP-1B are resistant to both diabetes and obesity. These data suggest that inhibitors of PTP-1B may be beneficial in the treatment of Type 2 diabetes.

Thus, inhibitors of PTP-1B improve insulin-sensitivity, and demonstrate utility in controlling or treating Type 1 and Type 2 diabetes, in improving insulin sensitivity, and in improving glucose tolerance. Such inhibitor compounds and compositions may also prove useful in treating or preventing cancer, neurodegenerative diseases and the like.

### SUMMARY

Provided herein are compounds, pharmaceutical compositions, and methods for the modulation of tyrosine phosphatase activity, and particularly PTP-1B activity. Such compounds, compositions and methods will find use in the treatment of conditions and diseases caused by dysfunctional signal transduction.

In one aspect, provided herein is a method for inhibiting protein tyrosine phosphatase activity which comprises administering to a mammal an effective amount of a compound having the formula: 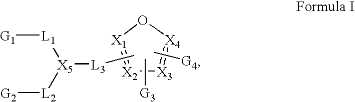
wherein:
X₁, X₂, X₃, and X₄ are each, independently, N or C, and
X₅ is CH, N, S(═O)ₙ (where n=0, 1 or 2), or O, with the following provisos:
a) at least 1, and at most 3 of the atoms X₁-X₄ must be N;
b) If there is a substituent G3 then the atom Xₙ to which it is attached must be carbon;
c) If there is a substituent G4 then the atom Xₙ to which it is attached must be carbon;
d) The scaffold X₁X₂X₃X₄O must be a stable heteroaromatic ring; and
e) If X₅ is S(═O)ₙ or O, then L₂ =no substituent and G₂ =no substituent;

G₁, G₂, G₃ and G₄ are substituent moieties as hereinafter more fully defined, including the following:

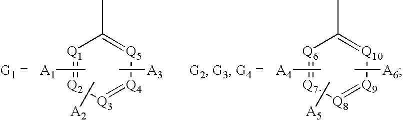

L₁, L₂, and L₃ are linkers as hereinafter more fully defined;

Q₁ through Q₁₇ are independently selected from no bond (direct link), C, N, S, and O, with the proviso that the resulting combination of atoms is a chemically stable cyclic and/or (hetero)aromatic ring system; and

appended A₁ through A₆ substituent groups can be combined to form stable mono- or bicyclic-fused alicyclic, heterocyclic and/or (hetero)aromatic rings.

In another aspect, a method for inhibiting protein tyrosine phosphatase activity is provided, which includes administering to a mammal a compound having the formula: 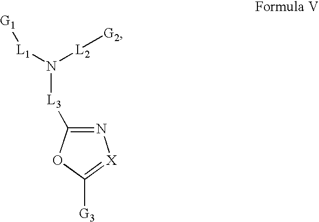
or a pharmaceutically acceptable salt thereof. Compounds according to Formula V can also find use in the treatment of various diseases such as obesity, diabetes, cancer, and neurodegenerative diseases.
In Formula V, L₁, L₂, and L₃ can be, independently, a bond or CH₂;
X is CR7 or N, where R7 is H or C1-C3 alkyl;
G₁ is H or a phenyl ring, where the phenyl ring is optionally substituted with one or more moieties selected from the group consisting of: phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester, where acyl is C₂-C₇ alkanoyl or C4-C7 cycloalkanoyl, phosphonodifluoromethyl mono-alkoxycarbonyloxymethyl ester, where alkoxy is C1-C6 or C3-C6 cycloalkoxy, 2-carboxyethenyl optionally substituted with 1-2 fluorines or methyl groups, carboxymethoxy, carboxy —C2-C4-alkyl optionally further substituted with 1-4 halogen atoms or 1-4 methyl groups, Cl, Br, F, CN, OH, CH₃, and ethynyl;
G₂ is H, C₁-C₃ alkyl, or a phenyl or pyridyl ring, where the phenyl or pyridyl ring is optionally and independently substituted with 1, 2, or 3 of the following moieties: Cl, F, Br, carboxy, methoxycarbonyl, OCF₃, OCHF₂, C₁-C₃ alkyl, and C₁-C₃-alkylsulfonyl;
G₃ is H, C₁-C₃ alkyl, or a phenyl or pyridyl ring, where the phenyl or pyridyl ring is optionally substituted with:
   (i) F, Cl, Br, CF₃, OR, methoxycarbonyl, carboxy, (CRR₁)ₙCO₂R, CF₂CO₂R, O(CRR₁)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR₁)CO₂R, SO₂NRR1, C₁-C₃-alkylsulfonyl, or CF₂P(═O)(OR)(OR1);
   (ii) phenyl, where the phenyl is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1);
   (iii) phenoxy, where the phenoxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1); or
   (iv) benzyloxy, where the benzyloxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1),
where m=0 to 6 and n=0 to 2; and
   where R and R1 are independently selected from hydrogen, an alkyl group of 1 to 6 carbon atoms, where the alkyl group is unsubstituted or mono-, di- or tri-substituted with 1 to 3 substituents selected from the group consisting of Y₁, Y₂, Y₃, an aryl group, —OC(R2R3)OC(═O)R4, and —OC(R2R3)OC(═O)OR4, or where R and R1 are joined to form a 4-8 membered cycloalkyl, cycloalkenyl, cycloalkynyl, or heterocyclic ring;
   where R2, R3 and R4 are independently selected from (i) and (ii) as follows:
   (i) H, C₁-C₇ alkyl, alkenyl of 2 to 6 carbon atoms, where the alkenyl group is unsubstituted or mono-, di- or tri-substituted with 1 to 3 substituents selected from the group consisting of Y₁, Y₂, and Y₃, alkynyl of 2 to 6 carbon atoms, where the alkynyl group is unsubstituted or mono-, di- or tri-substituted with 1 to 3 substituents selected from the group consisting of Y₁, Y₂, and Y₃, cycloalkyl of 3 to 8 carbon atoms, where the cycloalkyl group is unsubstituted or mono-, di- or tri-substituted with 1 to 3 substituents selected from the group consisting of Y₁, Y₂, and Y₃, aryl of 6 to 14 carbon atoms, where the aryl group is unsubstituted or mono-, di- or tri-substituted with 1 to 3 substituents selected from the group consisting of Y₁, Y₂, and Y₃, linked biaryl or heterobiaryl groups of 10 to 20 atoms featuring two aromatic or heteroaromatic ring systems linked through a single bond, with the ring atoms selected from carbon and heteroatoms, where the heteroatoms are selected from oxygen, nitrogen, and sulfur, and where the linked biaryl or heterobiaryl group is unsubstituted or mono-, di- or tri-substituted with 1 to 3 substituents selected from the group consisting of Y₁, Y₂, and Y₃, aralkyl of 7 to 16 carbon atoms, where the aralkyl is unsubstituted or mono-, di- or tri-substituted with 1 to 3 substituents selected from the group consisting of Y₁, Y₂, and Y₃, monocyclic-heteroaryl or bicyclic-heteroaryl having 5 to 14 ring atoms with the ring atoms selected from carbon and heteroatoms, where the heteroatoms are selected from oxygen, nitrogen, and sulfur, and where the monocyclic-heteroaryl or bicyclic heteroaryl group is unsubstituted or mono-, di- or tri-substituted with 1 to 3 substituents selected from the group consisting of Y₁, Y₂, and Y₃, and a heteroaralkyl group of 5 to 14 ring atoms with the ring atoms selected from carbon and heteroatoms, where the heteroatoms are selected from oxygen, nitrogen, and sulfur, where the heteroaralkyl is unsubstituted or substituted on the alkyl chain and which is unsubstituted on the ring or mono-, di- or tri-substituted on the ring with 1 to 3 substituents selected from the group consisting of Y₁, Y₂, and Y₃; or
   (ii) R2 and R3, and/or R3 and R4, and/or R2 and R4 are joined to form a 4-8-membered cycloalkyl, cycloalkenyl, cycloalkynyl, or heterocyclic ring, and the other of R2, R3, and R4, when not joined in a ring, is selected as in (i) above;

and wherein Y1, Y2, and Y3 are independently selected from (i) or (ii) as follows:
(i) R5, (CR5R6)ₙOR5, OH, (CR5R6)ₙNR5R6, C(═NR5)NR5R6, C(═NOR5)NR5R6, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5,C(═O)R5,—(CR5R6)ₙC(═O)R5, (CF₂)ₙC(═O)R5, (CFR5)ₙC(═O)R5, tetrazolyl (Tzl), (CR5R6)ₙTzl, (CF₂)ₙTzl, (CFR5)ₙTzl, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, (CR5R6)ₙC(═O)C(═O)OR5, (CR5R6)ₙCH(OR5)C(═O)OR5, C(R5)═C(R6), C(═O)OR5, C(R5)═C(R6)-Tzl, (CR5R6)ₙP(═O)(OH)₂, (CR5R6)ₙP(═O)(OR5)(OR6), P(═O)(OR5)[(OCR5R6)OC(═O)R5], P(═O)(OR5)[(OCR5R6)OC(═O)OR5], P(═O)[(OCR5R6)OC(═O)R5)][(OCR5R6)OC(═O)R5], P(═O)[(OCR5R6)OC(═O)OR5)][(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5═CR5—P(═O)(OR5)(OR6), CR5═CR5—P(═O)(Me)(OR5), CC—P(═O)(OR5)(OR6), (C═O)P(═O)(OR5)(OR6), (C═O)P(═O)(Me)(OR5), (C═O)P(═O)(CF₃)(OR5), (CR5OR6)ₙP(═O)(OR5)(OR6), (CR5OR6)ₙP(═O)(Me)(OR5), (CR5OR6)ₙP(═O)(CF₃)(OR5), O(CR5R6), C(═O)OR5, O(CF₂)ₙC(═O)OR5, OCH[C(═O)OR5]₂, O(CR5R6)ₙCH[C(═O)OR5]₂, OCF[C(═O)OR5]₂, O(CR5R6)ₙC(═O)C(═O)OR5, O(CF₂)ₙC(═O)C(═O)OR5, O(CR5R6)ₙTzl, O(CF₂)ₙTzl, OCH(Tzl)₂, O(CF₂)ₙP(═O)(OR5)(OR6), O(CF₂)ₙP(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF[P(═O)(Me)(OR5)]₂, SO₃H, —(CR5R6)ₙSO₃H, S(O)ₙR5, SCF₃, SCHF₂, SO₂CF₃, SO₂Ph, (CR5R6)ₙS(O)ₙR5, (CR5R6)ₙS(O)₂CF₃, (CR5R6)ₙSO₂NR5R6, (CR5R6)ₙSO₂NR5C(═O)(Me, CF₃), (CF₂)ₙSO₃H, (CFR5)ₙSO₃H, and (CF₂)ₙSO₂NR5R6, where n=0-2, and where R5 and R6 can be H, C1-C6 alkyl, C2-C6 alkenyl, C2-C6 alkynyl, a C3-C8 cycloalkyl ring, or a 5-7 membered heterocyclic ring; or
(ii) Y₁ and Y₂, and/or Y₁ and Y₃, and/or Y₂ and Y₃ are selected together to be (CR5R6)₂₋₆, —O[C(R8)(R9)]ᵣO— or —O[C(R8)(R9)]ᵣ₊₁—, where r is an integer from 1 to 4 and R8 and R9 are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroarylalkyl of 5 to 14 ring atoms, and the other of Y1, Y2, and Y3, when not selected as in (ii), is selected as in (i) above.

Also provided herein are compounds (e.g., according to Formula I and/or Formula V) and compositions useful for inhibiting protein tyrosine phosphatase activity, particularly PTP-1B activity, and for treating or preventing diseases such as obesity and diabetes.

All patents and patent applications cited in this specification are hereby incorporated by reference as if they had been specifically and individually indicated to be incorporated by reference.

### DETAILED DESCRIPTION

A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, protein tyrosine phosphatase (PTP) refers to an enzyme of the PTP class, including enzymes that are both tyrosine-specific and dual-specific in their phoshpatase activity. In one embodiment, such phosphatases encompass both transmembrane receptor-like PTPs (RPTPs) as well as soluble cytosolic proteins. RPTPs include small glycosylated segments (e.g., RPTPa, RPTPe), tandem repeats of immunoglobulin-like and/or fibronectin type III domains (e.g., LAR) or carbonic anhydrase like domains (e.g., RPTPg, RPTPb). Intracellular or cytoplasmic PTPs (CPTPs), include PTP1B or PTP-1B, PTP1C and PTP1D, and typically contain a single catalytic domain flanked by several types of modular conserved domains.

As used herein, protein tyrosine phosphatase 1B (PTP-1B) refers to a 37-kD protein comprised of a single domain, is topologically organized into 8 alpha helices and 12 beta sheets. See, e.g., Jia, Z., Barford, D., Flint, A. J., and N. K. Tonks (1995) Science 268:1754-1758; Pannifer A., Flint A., Tonks N., and Barford D. (1998) The Journal of Biological Chemistry 273:10454-10462; Charbonneau et al., 1989, Proc. Natl. Acad. Sci. USA 86:5252-5256; Goldstein, 1993, Receptor 3:1-15.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl and cycloalkyl. Pharmaceutically acceptable enol esters include, but are not limited to, derivatives of formula C═C(OC(O)R) where R is alkyl, alkenyl, alkynyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which a-synuclein fibril formation is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of ═-synuclein fibril formation, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392). Other prodrugs are described elsewhere herein.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi- cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fuised to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quatemized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO—in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein,“oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur orsubstituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fuised, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Q1.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —NHC(O)NH—. “Thioureido” refers to the divalent group —NHC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Compounds, Compositions and Methods

Provided herein are compounds, compositions, and methods for the inhibition of tyrosine phosphatase activity. Such compounds, compositions and methods will find use in the treatment of conditions and diseases caused by dysfunctional signal transduction.

The compounds provided herein are generally characterized as nitrogen-containing organooxygen compounds, e.g., according to Formula I or Formula V, as shown below, and their pharmaceutically acceptable salts. It should be noted that a compound provided herein may contain one or more asymmetric centers and thus can give rise to optical isomers and diastereomers. The scope of the present disclosure includes all possible isomers and diastereomers, as well as their racemic and resolved, enantiomerically pure forms. Certain of the present compounds contain olefinic double bonds and, unless specified to the contrary, the compounds provided herein include both the E and Z geometric isomeric forms.

1. Formula I

Accordingly, in one aspect, provided herein is a method for inhibiting protein tyrosine phosphatase activity, which comprises administering to a mammal an effective amount of a compound having the formula: 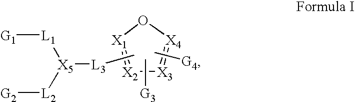
where:
X₁, X₂, X₃, and X₄ are each, independently, N or C, and
X₅ is CH, N, S(═O), (where n=0, 1 or 2), or O, with the following provisos:
a) at least 1, and at most 3 of the atoms X₁-X₄ must be N;
b) If there is a substituent G3 then the atom Xₙ to which it is attached must be carbon;
c) If there is a substituent G4 then the atom Xₙ to which it is attached must be carbon;
d) The scaffold X₁X₂X₃X₄O must be a stable heteroaromatic ring; and
e) If X₅ is S(═O)ₙ or O, then L₂=no substituent and G₂=no substituent;
where G₁, G₂, G₃ and G₄ are substituent moieties as hereinafter more fully defined, including the following:

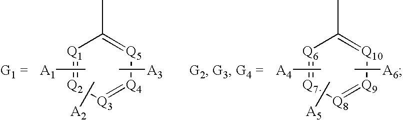

where L₁, L₂, and L₃ are linkers as hereinafter more fully defined;

where Q₁ through Q₁₇ are independently selected from no bond (direct link), C, N, S, and O, with the proviso that the resulting combination of atoms is a chemically stable cyclic and/or (hetero)aromatic ring system; and

where appended A₁ through A₆ substituent groups can be combined to form stable mono- or bicyclic-fused alicyclic, heterocyclic and/or (hetero)aromatic rings.

Also provided herein are compounds, e.g., according to Formula I, and compositions useful for inhibiting protein tyrosine phosphatase activity, particularly PTP-1B activity.

In the above Formula I, the defined linkers and groups will be in accordance with the following description, where bonds are shown only where required for clarity. All combinations of the following groups are within the scope of the present disclosure.

a. Linkers

L₁, L₂ and L₃ are independently selected from the following: no bond (i.e. direct link to G₁, G₂, or X₅), (CRR1)ₘ, CF₂, CF₂CF₂, C(═O), C(═O)C(═O), C(═O)(CRR1)ₘ, (CRR1)ₘC(═O)(CRR1)ₘ, C(═O)O(CRR1)ₘ, (CRR1)ₘC(═O)O, N(R), —C(═O)N(R)N(R1), N(R)SO₂N(R1), C(═O)N(R), N(R)C(═O)N(R1), O, OC(═O)N(R), P(═O)(OR), P(═O)(NR), P(═S)(OR), P(═S)(NR), SO₂, S(═O)ₙ(CRR1)ₘ, (CRR1)ₘS(═O)ₙ(CRR1)ₘ, where m=0-6 and n=0-2, S(═O)(═NR), S(═NR)(═NR1), SO₂NR, C₁-C₇ alkyl, C₂-C₇-alkenyl, C₂-C₇-alkynyl, C₃-C₇ cycloalkyl, aryl of from about 6 to about 10 carbon atoms, heteroaryl containing from about 5 to about 10 atoms (selected from C, N, O), with the proviso that the bonds from L₁ to X₅ and from L₂ to X₅ are chemically stable in aqueous solutions.

b. Groups

R and R1 are independently selected from hydrogen and alkyl of 1 to about 6 carbon atoms and which is unsubstituted or mono-, di- or tri-substituted with 1 to 3 substituents selected from the group consisting of Y₁, Y₂, and Y₃, —OC(R2R3)OC(═O)R4, and —OC(R2R3)OC(═O)OR4.

R2, R3 and R4 are independently selected from H, C₁-C₇ alkyl, R2, R3 and R4 can be combined to form a 5-7-membered ring, alkenyl of 2 to about 6 carbon atoms and which is unsubstituted or mono-, di- or tri-substituted with 1 to 3 substituents selected from the group consisting of Y₁, Y₂, and Y₃, alkynyl of 2 to about 6 carbon atoms and which is unsubstituted or mono-, di- or tri-substituted with 1 to 3 substituents selected from the group consisting of Y₁, Y₂, and Y₃, cycloalkyl of 3 to about 8 carbon atoms and which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, linked biaryl and heterobiaryl of about 10 to 20 atoms featuring two (hetero)aromatic ring systems linked through a single bond, with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, monocyclic-heteroaryl and bicyclic-heteroaryl, each of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃; R and R1 can be joined together to form an alicyclic or heterocyclic ring.

Examples of aryl groups include phenyl, naphthyl, tetrahydronaphthyl, indenyl, indanyl, anthracenyl and fluorenyl ring systems.

Examples of monocyclic heteroaryl, e.g. heteroaryl of about 5 to 6 ring atoms include furyl, thienyl, pyrrolyl, pyrazolyl, imidazolyl, triazolyl, oxazolyl, isoxazolyl, oxadiazolyl, thiazolyl, isothiazolyl, thiadiazolyl, pyridyl, pyridazinyl, pyrimidinyl, pyrazinyl, triazinyl (1,3,5- and 1,2,4-isomers) and tetrazinyl ring systems.

Examples of bicyclic heteroaryl, e.g. heteroaryl of about 8 to 10 ring atoms, include benzothienyl, benzofuranyl, indolyl, benzimidazoyl, indazolyl, benzotriazolyl, benzothiazolyl, isobenzothiazolyl, benzoxazolyl, isobenzoxazolyl, quinolinyl, isoquinolinyl, cinnolinyl, phthalazinyl, quinazolinyl, quinoxalinyl, and stable partially reduced congeners, such as, e.g., dihydrobenzofuranyl, indolinyl, dihydrobenzothienyl, dihydrobenzopyranyl (chromane), iso-dihydro-benzopyranyl (isochromane), dihydrobenzothiopyranyl (thiochroman), iso-dihydrobenzothiopyranyl (isothiochroman), tetrahydroquinolinyl, tetrahydroisoquinolinyl and similar ring systems.

Examples of linked biaryl and heterobiaryl include 2-phenylphenyl, 3-phenylphenyl, 4-phenylphenyl, phenylnaphthyl, bithienyl, thienyloxazolyl, phenylpyridyl, thiazolylpyridyl, phenylpyrimidinyl, phenyltriazinyl, phenylthienyl, naphthylfuranyl and heterocyclic analogs of these in which C is replaced by N, C═C is replaced by S, and/or C═C is replaced by O.

As indicated above, R and R1 are independently and optionally substituted with 1 to 3 substituents Y₁, Y₂, and Y₃ which can be selected from the group consisting of R5, (CR5R6)ₙOR5, OH, (CR5R6)ₙNR5R6, C(═NR5)NR5R6, C(═NOR5)NR5R6, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5, C(═O)R5,—(CR5R6)ₙC(═O)R5, (CF₂)ₙC(═O)R5, (CFR5)ₙC(═O)R5, tetrazolyl (Tzl), (CR5R6)ₙTzl, (CF₂)ₙTzl, (CFR5)ₙTzl, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, (CR5R6)ₙC(═O)C(═O)OR5, (CR5R6)ₙCH(OR5)C(═O)OR5, C(R5)═C(R6), C(═O)OR5, C(R5)═C(R6)-Tzl, (CR5R6)ₙP(═O)(OH)₂, (CR5R6)ₙP(═O)(OR5)(OR6), P(═O)(OR5)[(OCR5R6)OC(═O)R5], P(═O)(OR5)[(OCR5R6)OC(═O)OR5], P(═O)[(OCR5R6)OC(═O)R5)][(OCR5R6)OC(═O)R5], P(═O)[(OCR5R6)OC(═O)OR5)][(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5═CR5—P(═O)(OR5)(OR6), CR5═CR5—P(═O)(Me)(OR5), CC—P(═O)(OR5)(OR6), (C═O)P(═O)(OR5)(OR6), (C═O)P(═O)(Me)(OR5), (C═O)P(═O)(CF₃)(OR5), (CR5OR6)ₙP(═O)(OR5)(OR6), (CR5OR6)ₙP(═O)(Me)(OR5), (CR5OR6)ₙP(═O)(CF₃)(OR5), O(CR5R6)ₙC(═O)OR5, O(CF₂)ₙC(═O)OR5, OCH[C(═O)OR5]₂, O(CR5R6)ₙCH[C(═O)OR5]₂, OCF[C(═O)OR5]₂, O(CR5R6)ₙC(═O)C(═O)OR5, O(CF₂)ₙC(═O)C(═O)OR5, O(CR5R6)ₙTzl, O(CF₂)ₙTzl, OCH(Tzl)₂, O(CF₂)ₙP(═O)(OR5)(OR6), O(CF₂)ₙP(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF [P(═O)(Me)(OR5)]₂, SO₃H, —(CR5R6)ₙSO₃H, S(O)ₙR5, SCF₃, SCHF₂, SO₂CF₃, SO₂Ph, (CR5R6)ₙS(O)ₙR5, (CR5R6)ₙS(O)₂CF₃, (CR5R6)ₙSO₂NR5R6, (CR5R6)ₙSO₂NR5C(═O)(Me, CF₃), (CF₂)ₙSO₃H, (CFR5)ₙSO₃H, (CF₂)ₙSO₂NR5R6, wherein n=0-2, and R5 and R6 can be H, C1-C6 alkyl, C2-C6 alkenyl, C2-C6 alkynyl, a C3-C8 cycloalkyl ring, or a 5-7 membered heterocyclic ring, or Y₁, Y₂ and/or Y₃ may also be selected together to be (CR5R6)₂₋₆ and substituted variants thereof, —O[C(R8)(R9)]ᵣO— or —O[C(R8)(R9)]ᵣ₊₁—, wherein r is an integer from 1 to 4 and R8 and R9 are independently selected from the group consisting of hydrogen, alkyl of 1 to about 12 carbon atoms, aryl of about 6 to about 14 carbon atoms, heteroaryl of about 5 to about 14 ring atoms, aralkyl of about 7 to about 15 carbon atoms, and heteroarylalkyl of about 5 to about 14 ring atoms.

G₁, G₂ and G₃ are independently selected from the following:
(i) H, alkyl of 1 to about 6 carbon atoms and which is unsubstituted or mono-, di- or tri-substituted with 1 to 3 substituents selected from the group consisting of Y₁, Y₂, and Y₃, alkenyl of 2 to about 6 carbon atoms and which is unsubstituted or mono-, di- or tri-substituted with 1 to 3 substituents selected from the group consisting of Y₁, Y₂, and Y₃, alkynyl of 2 to about 6 carbon atoms and which is unsubstituted or mono-, di- or tri-substituted with 1 to 3 substituents selected from the group consisting of Y₁, Y₂, and Y₃, cycloalkyl of 3 to about 8 carbon atoms and which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃;
(ii) P(═O)(OR)(OR1), including P(═O)(OH)₂, P(═O)(OH)(OCH₃), P(═O)(OH)(OC₂H₅), P(═O)(OR)(OR1), P(═O)(OR)[(OCRR1)OC(═O)R], P(═O)(OR)[(OCRR1)OC(═O)OR], P(═O)[(OCRR1)OC(═O)R)][(OCRR1)OC(═O)R], P(═O)[(OCRR1)OC(═O)OR)][(OCRR1)OC(═O)OR], P(═O)(Me)(OR), P(═O)(CF₃)(OR), P(═O)(Me)(NHR), P(═O)(NHR)(OR), P(═O)(NHR)(NHR1), CR═CR-P(═O)(OR)(OR1), CR═CR-P(═O)(Me)(OR), CR═CR-P(═O)(CF₃)(OR), CR═CR-P(═O)(Me)(NHR), CR═CR-P(═O)(NHR)(OR), CR═CR-P(═O)(NHR)(NHR1), [CH(OH)]_{q}P(═O)(OR)(OR1), [CH(OH)]_{q}P(═O)(Me)(OR1), [CH(OH)]_{q}P(═O)(CF₃)(OR1), CC-P(═O)(OR)(OR1), CC-P(═O)(Me)(OR), CC-P(═O)(CF₃)(OR), CC-(CF₂)_{q}-P(═O)(OR)(OR1), CC-(CF₂)_{q}-P(═O)(Me)(OR1), CC-(CF₂)_{q}-P(═O)(CF₃)(OR1), [CH(OH)]_{q}CF₂P(═O)(OR)(OR1), [CH(OH)]_{q}(CF₂)_{q}P(═O)(Me)(OR1), [CH(OH)]_{q}(CF₂)_{q}P(═O)(CF₃)(OR1), (CF₂)_{q}P(═O)(OR)(OR1), (CF₂)_{q}P(═O)(Me)(OR), (CF₂)_{q}P(═O)(CF₃)(OR), (CF2)_{q} P(═O)(Me)NHR, (CF₂)_{q}P(═O)(NHR)(OR), (CFR)_{q}P(═O)(OR)(OR1), (CFR)_{q}P(═O)(Me)(OR), (CFR)_{q}P(═O)(CF₃)(OR), (CFR)_{q}P(═O)(Me)NHR, (CF₂)_{q}P(═O)(NHR)(OR), CF═CF-P(═O)(OR)(OR1), CF═CF-P(═O)(Me)(OR), CF═CF-P(═O)(CF₃)(OR), CF═CF-P(═O)(Me)(NHR), CF═CF-P(═O)(NHR)(OR), CH═C[P(═O)(OR)₂]₂, CF═C[P(═O)(OR)₂]₂, CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)₂]₂, CF[P(═O)(OR)(OR1)]₂, CF[P(═O)(Me)(OR)]₂, CF[P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, and C(OH)[P(═O)(NHR)(OR)]₂, wherein q=1 to 3;
(iii) SO₃H, SO₂NH₂, SO₂NHTzl, SO₂NHC(═O)(Me, CF₃), SO₂NHC(═O)NH₂, (CRR1)_{q}SO₃H, (CRR1)_{q} SO₂NH₂, (CRR1)_{q}SO₂NHTzl, (CRR1)_{q}SO₂NHC(═O)(Me, CF₃), (CRR1)_{q}SO₂NHC(═O)NH₂, SO₂NHCRR1C(═O)C(═O)OR, SO₂CF₃, CH(SO₂Me)₂, CH(SO₂CF₃)₂, SO₂CRR1C(═O)OR, SO₂CH[C(═O)OR]₂, (CRR1)_{q}SO₂NHCRR1C(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tzl, SO₂(CRR1)_{q}P(═O)(OR)₂, SO₂(CF₂)_{q}C(═O)OR, SO₂(CF₂)_{q}Tzl, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO2NHC(═O)(Me, CF₃), and C(═NSO₂CF₃)(NHSO₂CF₃);
(iv) NHC(═O)C(═O)OR, NHC(═O)C(═O)O(CRR1)OC(═O)R, NHC(═O)C(═O)O(CRR1)OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTzl, NHC(═O)Tzl, NHSO₂Tzl, NH(CF₂)_{q}Tzl, NHSO₂(CRR1)_{q}C(═O)OR, NHSO₂(CF₂)_{q}C(═O)OR, (CRR1)_{q}NO₂,(CF₂)_{q}NO₂, CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR]CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), and NH(CFR)_{q}P(═O)(CF₃)(OR);
(v) C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂ CH═C[C(═O)OR]₂, CF═C[C(═O)OR]₂, C(R4)═C(R5)(R6), (where R4, R5=H, Me, anionic groups, including OH, SO₃H, carboxyl, tetrazole, 3-hydroxy-isoxazol-5-yl, C(═O)NHSO₂(Me, CF₃), C(═O)NHC(═O)(Me, CF₃), SO₂NHC(═O)(Me, CF₃), R6═H, F), C(═O)C(═O)OR, C(═O)CH[C(═O)OR]₂, C(═O)CH(Tzl)₂, C(═O)CRR1C(═O)(Me, CF₃, Ph), C(═O)CRR1SO₂(Me, CF₃, Ph), (CRR1)_{q}C(═O)C(═O)OR, (CF2)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, (CHF)_{q}C(═O)CF₃, (CF₂)_{q}C(═O)CF₃, (CHF)_{q}C(OR)₂CF₃, (CF₂)_{q}C(OR)₂CF₃, CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C[C(═O)OH]═NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tzl)₂, C(═O)N[CRR1P(═O)(OR)₂]₂, and C(═O)NHC(CRR1OR)₃;
(vi) Tzl, CR(Tzl)₂, (CRR1)_{q}Tzl, (CF₂)_{q}Tzl, (CFR)_{q}Tzl, CF(Tzl)₂, (CF₂)_{q}CF(Tzl)₂, (CF₂)_{q}CR(Tzl)₂, CR═CR-Tzl, CF═CH-Tzl, CH═CF-Tzl, CF═CF-Tzl, CH═C(Tzl)₂, CF═C(Tzl)₂, C(H, F)═C(Tzl)[P(═O)(OR)(OR1), P(═O)(Me)(OR), P(═O)(CF₃)(OR), P(═O)(Me)(NHR), P(═O)(NHR)(OR), and C(═O)OR];
(vii) OH, OR, O(CRR1)_{q}C(═O)OR, O(CF₂)_{q}C(═O)OR, OCH[C(═O)OR]₂, O(CRR1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTzl, O(CRR1)_{q}Tzl, O(CF₂)_{q}Tzl, OCH(Tzl)₂, O(CF₂)_{q}CF(Tzl)₂, O(CF₂)_{q}CR(Tzl)₂, OCF(Tzl)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(NHR)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR1)_{q}P(═O)(NHR)(OR), O(CRR1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF[P(═O)(OR)(OR1)]₂, OCF[P(═O)(Me)(OR)]₂, OCF[P(═O)(CF₃)(NHR)]₂, OCF[P(═O)(NHR)(OR)]₂, O(CRR1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(NHR), O(CRR1)_{q}(CF₂)_{q}P(═O)(NHR)(OR) ON═CH—C(═O)OR, and ON═C [C(═O)OR]CRR1C(═O)OR;
(viii) Heteroaryl, squarate, and related derivatives, including: 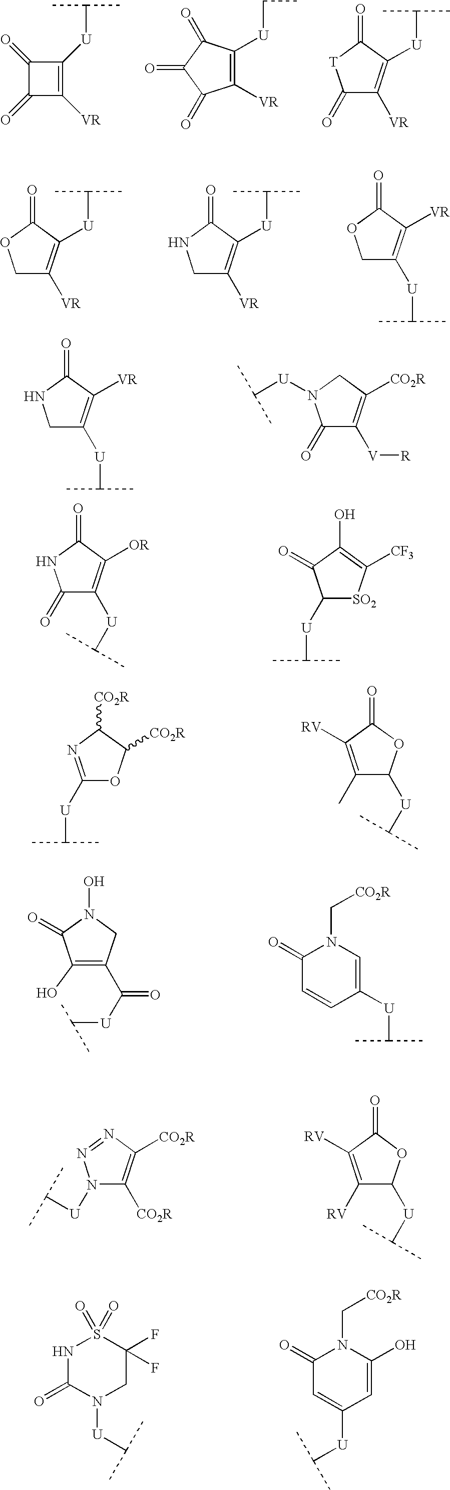 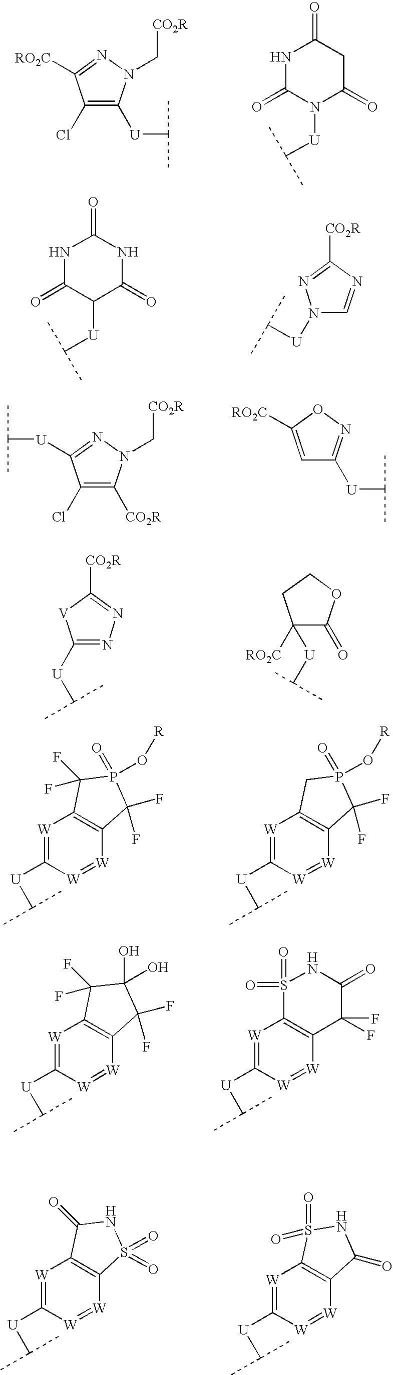
   wherein T═O, NR1, CR; U and V are chosen from direct link (CRR1)_{q},O, S, NR1; W═CR, N; and R and R1 are as defined above.

Certain compounds provided herein contain linkers, L₁ and L₂ each containing 1 to 2 atoms and G₁ groups with optionally substituted aromatic and heteroaromatic groups of the generic formulae:

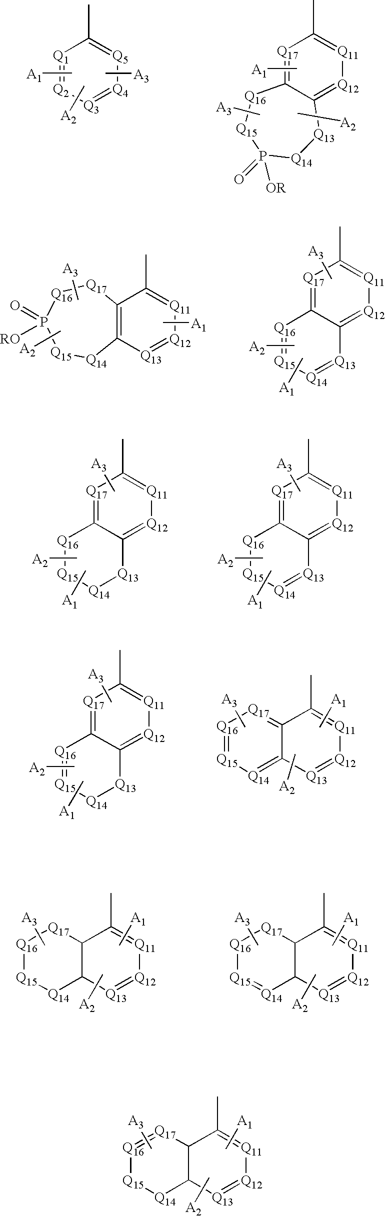

G₂ is selected from optionally substituted aromatic and heteroaromatic groups of the generic formulae: 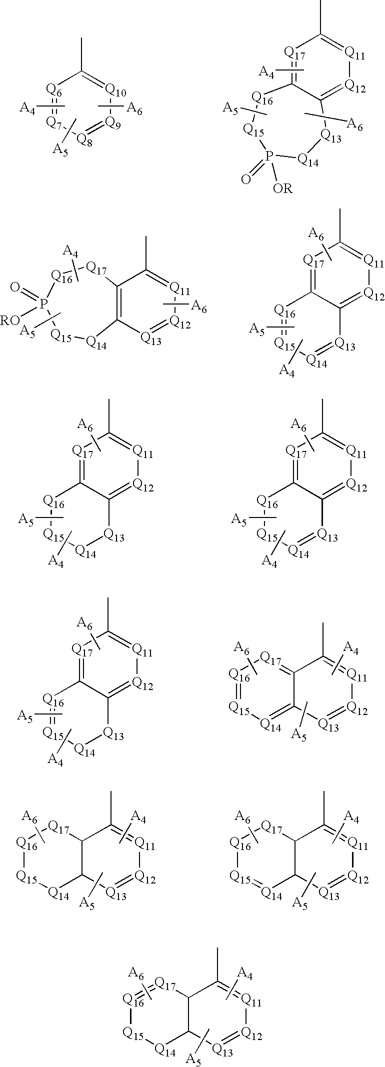
where A1-A6 are independently selected from:
(i) no substituent, H, F, Cl, Br, I, CF₃, CF₂CF₃, CH₂CF₃, CF₂CH₃, OH, OCF₃, OCHCl₂, CN, NO₂, C₁-C₆-alkyl which is unsubstituted or mono-, di- or tri-substituted with 1 to 3 substituents selected from the group consisting of Y₁, Y₂, and Y₃, C₂-C₆-alkenyl which is unsubstituted or mono-, di- or tri-substituted with 1 to 3 substituents selected from the group consisting of Y₁, Y₂, and Y₃, C₂-C₆-alkynyl which is unsubstituted or mono-, di- or tri-substituted with 1 to 3 substituents selected from the group consisting of Y₁, Y₂, and Y₃, C₁-C₆ alkoxy which is unsubstituted or mono-, di- or tri-substituted with 1 to 3 substituents selected from the group consisting of Y₁, Y₂, and Y₃, C₃-C₆ alkenyloxy which is unsubstituted or mono-, di- or tri-substituted with 1 to 3 substituents selected from the group consisting of Y₁, Y₂, and Y₃, C₃-C₆ alkynyloxy which is unsubstituted or mono-, di- or tri-substituted with 1 to 3 substituents selected from the group consisting of Y₁, Y₂, and Y₃, wherein Y₁, Y₂, and Y₃ are defined above, C₃-C₈-cycloalkyl which is unsubstituted or mono-, di- or tri-substituted with 1 to 3 substituents selected from the group consisting of Y₁, Y₂, and Y₃, aryl of about 6 to about 14 carbon atoms and which is unsubstituted or mono-, di- or tri-substituted with 1 to 3 substituents selected from the group consisting of Y₁, Y₂, and Y₃, aralkyl of about 7 to about 16 carbon atoms which is unsubstituted or mono-, di- or tri-substituted with 1 to 3 substituents selected from the group consisting of Y₁, Y₂, and Y₃, heteroaryl of about 5 to about 14 ring atoms with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃, and heteroaralkyl of about 5 to about 14 ring atoms with the ring atoms selected from carbon and heteroatoms, wherein the heteroatoms are selected from oxygen, nitrogen, and sulfur, which is unsubstituted or substituted on the alkyl chain and which is unsubstituted on the ring or mono-, di- or tri-substituted on the ring with 1 to 3 substituents selected from the group consisting of Y₁, Y₂, and Y₃; two adjacent A groups (e.g. A₁, A₂) may be joined together to form a fused alicyclic, heteroaromatic or aromatic ring. R, R1═H, C₁-C₆-alkyl, C₂-C₆-alkenyl, C₂-C₆-alkynyl; R, R1 may be joined together to form an alicyclic or heterocyclic ring; and one or more of A₁-A₆ may serve as a linking atom, such as O, S(O)₀₋₂, C(RR1), P(═O), P(═S), or N(R).

A₁-A₆ phosphorous-containing moieties include the following:
P(═O)(OR)(OR1), especially P(═O)(OH)₂, P(═O)(OH)(OCH₃), P(═O)(OH)(OC₂H₅), P(═O)(OR)[(OCRR1)OC(═O)R], P(═O)(OR)[(OCRR1)OC(═O)OR], P(═O)[(OCRR1)OC(═O)R)]₂, P(═O)[(OCRR1)OC(═O)OR)]₂, P(═O)(OR)(OR1), P(═O)(Me)(OR), P(═O)(CF₃)(OR), P(═O)(Me)(NHR), P(═O)(NHR)(OR), P(═O)(NHR)(NHR1), CR═CR-P(═O)(OR)(OR1), CR═CR-P(═O)(Me)(OR), CR═CR-P(═O)(CF₃)(OR), CR═CR-P(═O)(Me)(NHR), CR═CR-P(═O)(NHR)(OR), CR═CR-P(═O)(NHR)(NHR1), [CH(OH)]_{q}P(═O)(OR)(OR1), [CH(OH)]_{q}P(═O)(Me)(OR1), [CH(OH)]_{q} P(═O)(CF₃)(OR1), CC-P(═O)(OR)(OR1), CC-P(═O)(Me)(OR), CC-P(═O)(CF₃)(OR), CC-(CF₂)_{q}-P(═O)(OR)(OR1), CC-(CF₂)_{q}-P(═O)(Me)(OR1), CC-(CF₂)_{q}-P(═O)(CF₃)(OR1), [CH(OH)]_{q}CF₂P(═O)(OR)(OR1), [CH(OH)]_{q}(CF₂)_{q}P(═O)(Me)(OR1), [CH(OH)]_{q}(CF₂)_{q}P(═O)(CF₃)(OR1), (CF₂)_{q}P(═O)(OR)(OR1), (CF₂)_{q}P(═O)(Me)(OR), (CF₂)_{q}P(═O)(CF₃)(OR), (CF₂)_{q} P(═O)(Me)NHR, (CF₂)_{q}P(═O)(NHR)(OR), (CFR)_{q}P(═O)(OR)(OR1), (CFR)_{q}P(═O)(Me)(OR), -(CFR)_{q}P(═O)(CF₃)(OR), (CFR)_{q}P(═O)(Me)NHR, (CF₂)_{q}P(═O)(NHR)(OR), CF═CF-P(═O)(OR)(OR1), CF═CF-P(═O)(Me)(OR), CF═CF-P(═O)(CF₃)(OR), CF═CF-P(═O)(Me)(NHR), CF═CF-P(═O)(NHR)(OR), CH═C[P(═O)(OR)₂]₂, CF═C[P(═O)(OR)₂]₂,CH[P(═O)(OR)₂]₂, CH[P(═O)(OR)(OR1)]₂, CH[P(═O)(Me)(OR)]₂, CH[P(═O)(CF₃)(OR)]₂, CH[P(═O)(Me)NHR]₂, CH[P(═O)(NHR)(OR)]₂, CF[P(═O)(OR)₂]₂, CF[P(═O)(OR)(OR1)]₂, CF[P(═O)(Me)(OR)]₂, CF[P(═O)(CF₃)(OR)]₂, CF[P(═O)(Me)(NHR)]₂, CF[P(═O)(NHR)(OR)]₂, C(OH)[P(═O)(OR)(OR1)]₂, C(OH)[P(═O)(Me)(OR)]₂, C(OH)[P(═O)(CF₃)(OR)]₂, C(OH)[P(═O)(Me)NHR]₂, and C(OH)[P(═O)(NHR)(OR)]₂, wherein each q is, independently, 1 to 3 throughout.

A₁-A₆ sulfur-containing moieties include the following: SO₃H, SO₂NH₂, SO₂NHTzl, SO₂NHC(═O)(Me, CF₃), SO₂NHC(═O)NH₂, (CRR1)_{q}SO₃H, (CRR1)_{q} SO₂NH₂, (CRR1)_{q}SO₂NHTzl, (CRR1)_{q}SO₂NHC(═O)(Me, CF₃), (CRR1)_{q}SO₂NHC(═O)NH₂, SO₂NHCRR1C(═O)C(═O)OR, SO₂CF₃, CH(SO₂Me)₂, CH(SO₂CF₃)₂, SO₂CRR1C(═O)OR, SO₂CH[C(═O)OR]₂, (CRR1)_{q}SO₂NHCRRIC(═O)C(═O)OR, (CRR1)_{q}SO₂CF₃, (CRR1)_{q}CH(SO₂Me)₂, (CRR1)_{q}CH(SO₂CF₃)₂, (CRR1)_{q}SO₂CRR1C(═O)OR, (CRR1)_{q}SO₂CH[C(═O)OR]₂, SO₂(CRR1)_{q}C(═O)(Me, CF₃), SO₂(CRR1)_{q}SO₂(Me, CF₃), SO₂(CRR1)_{q}Tzl, SO₂(CRR1)_{q}P(═O)(OR)₂, SO₂(CF₂)_{q}C(═O)OR, SO₂(CF₂)_{q}Tzl, SO₂(CF₂)_{q}P(═O)(OR)₂, SO₂NHSO₂(CF₃, Me), (CF₂)_{q}SO₂(OH, NH₂), (CF₂)_{q}SO₂NHC(═O)(CF₃, Me), (CFR)_{q}SO₂(OH, NH₂), (CFR)_{q}SO₂NHC(═O)(CF₃, Me), CR═CRSO₂(OR, NHR), CR═CRSO₂NH₂, CR═CRSO₂NHC(═O)(Me, CF₃), and C(═NSO₂CF₃)(NHSO₂CF₃).

A₁-A₆ nitrogen-containing moieties include the following: NHC(═O)C(═O)OR, NHC(═O)C(═O)O(CRR1)OC(═O)R, NHC(═O)C(═O)O(CRR1)OC(═O)OR, NHC(═O)NRSO₂(Me, CF₃), NHSO₂(Me, CF₃), NHSO₂NRR1, NHSO₂NRC(═O)(Me, CF₃), NH(CRR1)_{q}C(═O)OR, NH(CF₂)_{q}C(═O)OR, NHTzl, NHC(═O)Tzl, NHSO₂Tzl, NH(CF₂)_{q}Tzl, NHSO₂(CRR1)_{q}C(═O)OR, NHSO₂(CF₂)_{q}C(═O)OR, (CRR1)_{q}NO₂,(CF₂)_{q}NO₂,CR═CRNO₂, CF═CFNO₂, (CRR1)_{q}NHSO₂(Me/CF₃), (CRR1)_{q}NHC(═O)(Me/CF₃), N(OCRR1C(═O)OR)CRR1C(═O)OR, NHCH[C(═O)OR] CH(OH)C(═O)OR, NHC(═O)[CH(OH)]_{q}C(═O)OR, NH(CRR1)_{q}P(═O)(OR)(OR1), NH(CRR1)_{q}P(═O)(Me)(OR), NH(CRR1)_{q}P(═O)(CF₃)(OR), NH(CF₂)_{q}P(═O)(OR)(OR1), NH(CF₂)_{q}P(═O)(Me)(OR), NH(CF₂)_{q}P(═O)(CF₃)(OR), NH(CFR)_{q}P(═O)(OR)(OR1), NH(CFR)_{q}P(═O)(Me)(OR), and NH(CFR)_{q}P(═O)(CF₃)(OR).

A₁-A6 carbonyl-containing moieties include the following: C(═O)OR, C(═O)O(CRR1)OC(═O)R, C(═O)O(CRR1)OC(═O)OR, C(═O)NHR, (CF₂)_{q}C(═O)OR, (CFR)_{q}C(═O)OR, CH[C(═O)OR]₂, CF[C(═O)OR]₂, CH═C[C(═O)OR]₂, CF═C[C(═O)OR]₂, C(R4)═C(R5)(R6), (where R4, R5 =H, Me, anionic groups, including OH, SO₃H, carboxyl, tetrazole, 3-hydroxy-isoxazol-5-yl, C(═O)NHSO₂(Me, CF₃), C(═O)NHC(═O)(Me, CF₃), SO₂NHC(═O)(Me, CF₃), R6 =H, F), C(═O)C(═O)OR, C(═O)CH[C(═O)OR]₂, C(═O)CH(Tzl)₂, C(═O)CRR1C(═O)(Me, CF₃, Ph), C(═O)CRR1SO₂(Me, CF₃, Ph), (CRR1)_{q}C(═O)C(═O)OR, (CF₂)_{q}C(═O)C(═O)OR, [CH(OR)]_{q}C(═O)OR, (CRR1)_{q}[CH(OR)]_{q}C(═O)OR, CR═CRCH(OR)C(═O)OR, C(OR)(CF₃)C(═O)OR, (CF₂)_{q}C(═O)CF₃, (CF₂)_{q}C(OH)₂CF₃, CHFC(═O)CF₃, CHFC(OR)₂CF₃, CH(OR)CH[C(═O)OR]₂, C(OR)[CRR1C(═O)OR]₂, (CF₂)_{q}C(OR)C(═O)OR, C(═O)C(═NOR)C(═O)(CH₃, OR), C(═O)CRR1C(═O)C(═O)OR, C(═NOR)C(═O)OR, CH═NOCRR1C(═O)OR, C[C(═O)OH]═NOCRR1C(═O)OR, CH(CN)NHC(═O)C(═O)OR, CH(NHCHO)C(═O)C(═O)OR, CH(NHCHO)C(OR)C(═O)OR, C(═O)N[CRR1C(═O)OR]OCRR1C(═O)OR, C(═O)N[CRR1C(═O)OR]₂, C(═O)N(CRR1Tzl)₂, C(═O)N[CRR1P(═O)(OR)₂]₂, and C(═O)NHC(CRRIOR)₃.

A₁-A6 tetrazole (Tzl)-containing moieties include the following: Tzl, CR(Tzl)₂, (CRR1)_{q}Tzl, (CF₂)_{q}Tzl, (CFR)_{q}Tzl, CF(Tzl)₂, (CF₂)_{q}CF(Tzl)₂, (CF₂)_{q}CR(Tzl)₂, CR═CR-Tzl, CF═CH-Tzl, CH═CF-Tzl, CF═CF-Tzl, CH═C(Tzl)₂, CF═C(Tzl)₂, C(H, F)═C(Tzl)[P(═O)(OR)(OR1), P(═O)(Me)(OR), P(═O)(CF₃)(OR), P(═O)(Me)(NHR), P(═O)(NHR)(OR), C(═O)OR].

A₁-A₆ oxygen-containing or oxygen-linked moieties include the following: OH, OR, O(CRR1)_{q}C(═O)OR, O(CF₂)_{q}C(═O)OR, OCH[C(═O)OR]₂, O(CRR1)_{q}CH[C(═O)OR]₂, OCF[C(═O)OR]₂, O(CRR1)_{q}CF[C(═O)OR]₂, O(CRR1)_{q}C(═O)C(═O)OR, O(CF₂)_{q}C(═O)C(═O)OR, O(CRR1)_{q}[CH(OR)]_{q}C(═O)OR, OCH[CRR1C(═O)OR]₂, OCF[CRR1C(═O)OR]₂, O(CF₂)_{q}CR(OR1)C(═O)OR, OTzl, O(CRR1)_{q}Tzl, O(CF₂)_{q}Tzl, OCH(Tzl)₂, O(CF₂)_{q}CF(Tzl)₂, O(CF₂)_{q}CR(Tzl)₂, OCF(Tzl)₂, O(CF₂)_{q}P(═O)(OR)(OR1), O(CF₂)_{q}P(═O)(Me)(OR), O(CF₂)_{q}P(═O)(CF₃)(OR), O(CF₂)_{q}P(═O)(Me)(NHR), O(CF₂)_{q}P(═O)(R)(OR), O(CF₂)_{q}P(═O)(NHR)(NHR1), O(CFR)_{q}P(═O)(OR)(OR1), O(CFR)_{q}P(═O)(Me)(OR), O(CFR)_{q}P(═O)(CF₃)(OR), O(CFR)_{q}P(═O)(Me)(NHR), O(CFR)_{q}P(═O)(NHR)(OR), O(CFR)_{q}P(═O)(NHR)(NHR1), O(CRR1)_{q}P(═O)(OR)(OR1), O(CRR1)_{q}P(═O)(Me)(OR), O(CRR1)_{q}P(═O)(CF₃)(OR), O(CRR1)_{q}P(═O)(Me)(NHR), O(CRR1)_{q}P(═O)(NHR)(OR), O(CRR1)_{q}P(═O)(Me)(OR), OCH[P(═O)(OR)(OR1)]₂, OCH[P(═O)(Me)(OR)]₂, OCH[P(═O)(Me)(OR)]₂, OCH[P(═O)(CF₃)(NHR)]₂, OCH[P(═O)(NHR)(OR)]₂, OCF[P(═O)(OR)(OR1)]₂, OCF[P(═O)(Me)(OR)1₂, OCF[P(═O)(CF₃)(NHR)]₂, OCF[P(═O)(NHR)(OR)]₂, O(CRR1)_{q}(CF₂)_{q}P(═O)(OR)(OR1), O(CRR1)_{q}(CF₂)_{q}P(═O)(Me)(OR), O(CRR1)_{q}(CF₂)_{q}P(═O)(CF₃)(OR), O(CRR1)_{q}(CF₂)_{q}P(═O)(Me)(HR), O(CRR1)_{q}(CF₂)_{q}P(═O)(NHR)(OR), ON═CH—C(═O)OR, and ON═C[C(═O)OR]CRR1C(═O)OR.

Other A₁-A₆ moieties contain the following heteroaryl, squarate, and related derivatives, including: 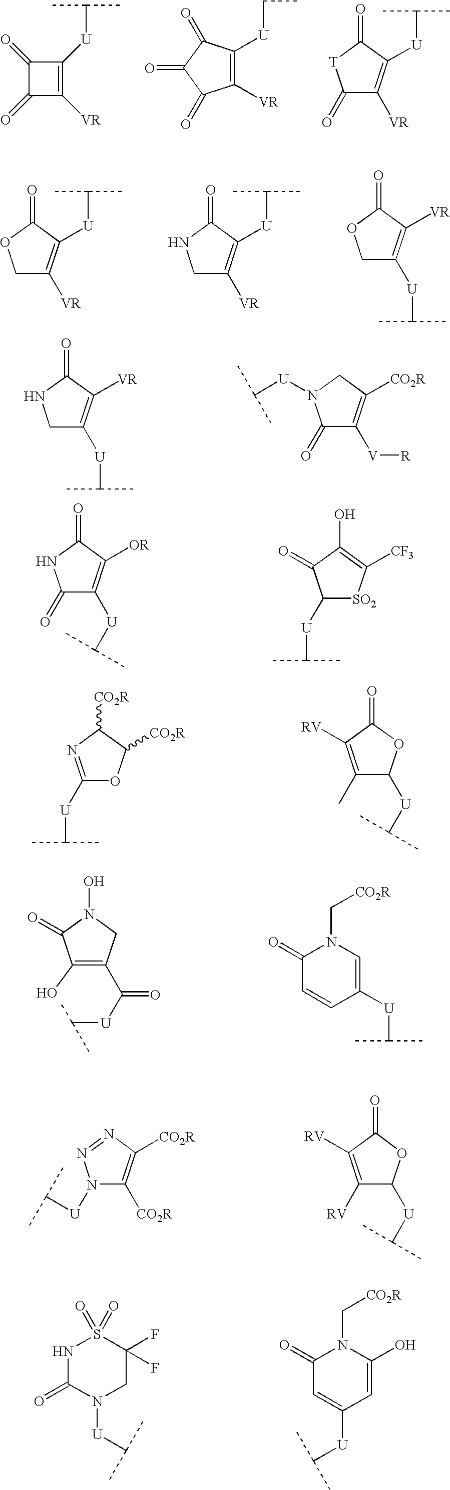 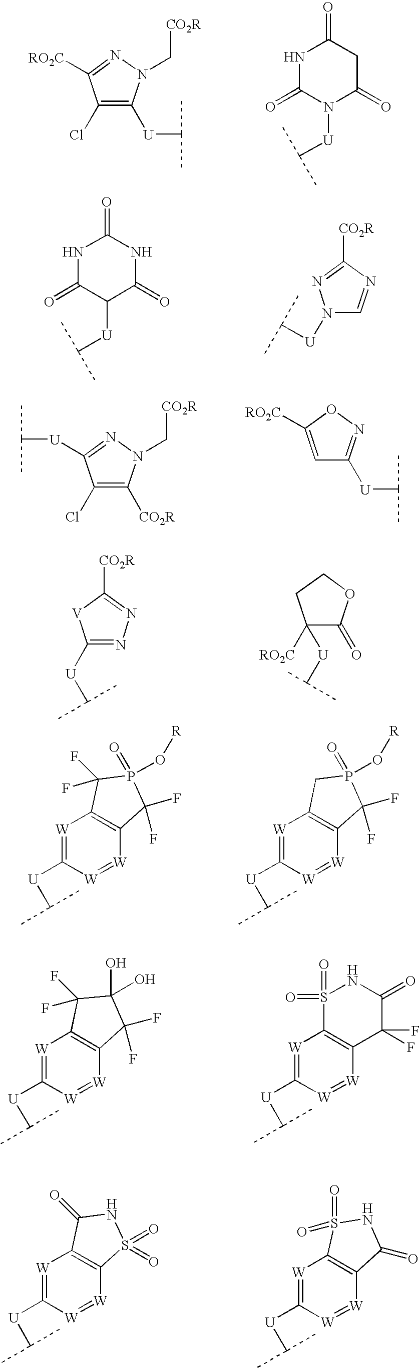
wherein T═O, NR1, CR; U and V are chosen from direct link, (CRR1)_{q}, O, S, NR1; W═CR, N.; and R and R1 are as defined above.

G₃ and G₄ can be independently selected from the group consisting of:
(1) alkyl of 1 to about 12 carbon atoms which is optionally unsubstituted or substituted with 1 to 3 substituents independently selected from the group consisting of Y₁, Y₂, and Y₃ as previously defined;
(2) alkyl of 1 to about 3 carbon atoms which is optionally substituted with cycloalkyl of about 3 to about 8 carbon atoms which is optionally substituted with 1 to 3 substituents independently selected from the group consisting of Y₁, Y₂, and Y₃;
(3) cycloalkyl of 3 to about 15 carbon atoms, which is unsubstituted or mono-, di-, or tri-substituted on the ring with 1 to 3 substituents selected from the group consisting of Y₁, Y₂, and Y₃;
(4) alkenyl of 2 to about 6 carbon atoms and which is optionally unsubstituted or mono-, di- or tri-substituted with 1 to 3 substituents selected from the group consisting of Y₁, Y₂, and Y₃;
(5) cycloalkenyl of 4 to about 8 carbon atoms and which is optionally unsubstituted or mono-, di- or tri-substituted with 1 to 3 substituents selected from the group consisting of Y₁, Y₂, and Y₃;
(6) alkyl of 1 to about 3 carbon atoms which is optionally substituted with cycloalkenyl of 4 to about 8 carbon atoms and which is optionally unsubstituted or mono-, di- or tri-substituted with 1 to 3 substituents selected from the group consisting of Y₁, Y₂, and Y₃;
(7) alkynyl of 2 to about 6 carbon atoms which is optionally unsubstituted or mono-, di- or tri-substituted with 1 to 3 substituents selected from the group consisting of Y₁, Y₂, and Y₃;
(8) alkynyl of 2 to about 6 carbon atoms which is optionally substituted with cycloalkyl of about 3 to about 8 carbon atoms, which is optionally substituted with 1 to 3 substituents independently selected from the group consisting of Y₁, Y₂, and Y₃;
(9) aryl of about 6 to about 14 carbon atoms which is optionally unsubstituted or mono-, di- or tri-substituted with 1 to 3 substituents selected from the group consisting of Y₁, Y₂, and Y₃;
(10) alkyl of 1 to about 3 carbon atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(11) alkenyl of 2 to about 6 carbon atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(12) alkynyl of 2 to about 6 carbon atoms which is optionally substituted with aryl of 6 to about 14 carbon atoms, which is optionally unsubstituted or mono-, di- or tri-substituted with 1 to 3 substituents selected from the group consisting of Y₁, Y₂, and Y₃;
(13) heteroaryl of about 5 to about 14 ring atoms with the ring atoms selected from carbon and heteroatoms, wherein the heteroatoms are selected from oxygen, nitrogen, and sulfur, and which is optionally unsubstituted or mono-, di- or tri-substituted with 1 to 3 substituents selected from the group consisting of Y₁, Y₂, and Y₃;
(14) alkyl of 1 to about 3 carbon atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(15) alkenyl of 2 to about 6 carbon atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(16) alkynyl of 2 to about 6 carbon atoms which is optionally substituted with heteroaryl of about 5 to about 14 ring atoms with the ring atoms selected from carbon and heteroatoms, wherein the heteroatoms are selected from oxygen, nitrogen, and sulfur, and which is which is optionally unsubstituted or mono-, di- or tri-substituted with 1 to 3 substituents selected from the group consisting of Y₁, Y₂, and Y₃;
(17) heterocyclo of 4 to about 10 ring atoms with the ring atoms selected from carbon and heteroatoms, wherein the heteroatoms are selected from the group consisting of oxygen, nitrogen, and S(O)ₘ, wherein m is 0, 1 or 2, which is unsubstituted or mono-, di-, or tri-substituted on the ring with 1 to 3 substituents selected from the group consisting of Y₁, Y₂, and Y₃;
(18) alkyl of 1 to about 3 carbon atoms which is optionally substituted with heterocyclo of 4 to about 10 ring atoms with the hetero-ring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(19) alkenyl of 2 to about 6 carbon atoms which is optionally substituted with heterocyclo of 4 to about 10 ring atoms with the hetero-ring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃;
(20) alkynyl of 2 to about 6 carbon atoms which is optionally substituted with heterocyclo of 4 to about 10 ring atoms with the hetero-ring atoms selected from carbon and heteroatoms, wherein the heteroatoms are selected from the group consisting of oxygen, nitrogen, and S(O)ₘ, wherein m i is 0, 1 or 2, which is unsubstituted or mono-, di-, or tri-substituted on the ring with 1 to 3 substituents selected from the group consisting of Y₁, Y₂, and Y₃; and
(21) biaryl and heterobiaryl of about 10 to 20 atoms featuring two (hetero)aromatic ring systems linked through a single, double , or triple bond, with the ring atoms selected from carbon and heteroatoms, wherein the heteroatoms are selected from oxygen, nitrogen, and sulfur, and which is unsubstituted or mono-, di- or tri-substituted with 1 to 3 substituents selected from the group consisting of Y₁, Y₂, and Y₃;
where Y₁, Y₂, and Y₃ are as previously defined.

### c. Specific Embodiments of Formula I

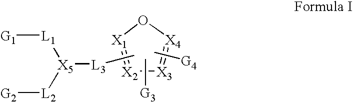

In certain cases, the following embodiments of Formula I are contemplated:
X₁=C;
X₂=N;
X₃=C or N;
X₄=C;
X₅=N;
L₁=bond or CH₂;
L₂=bond or CH₂;
L₃=bond or CH₂;
G₁=H or Phenyl ring, optionally substituted at the 3 or 4 position with phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester where acyl is C₂—C₇ alkanoyl or C4-C7 cycloalkanoyl, phosphonodifluoromethyl mono-alkoxycarbonyloxymethyl ester where alkoxy is C1-C6 or C3-C6 cycloalkoxy, 2-carboxyethenyl optionally substituted with 1-2 fluorines or methyl groups, carboxymethoxy, carboxyalkyl (C2-C4) optionally further substituted with 1-4 halogen atoms or 1-4 methyl groups. GI may also be optionally and independently substituted with Cl, Br, F, CN, OH, CH₃, or ethynyl; G₂=H, lower C₁-C₃ alkyl, or a phenyl or pyridyl ring, optionally substituted with 1-3 of the following substituents: Cl, F, Br, carboxy, methoxycarbonyl, OCF₃, OCHF₂, alkyl (CI-C3), and alkylsulfonyl (C₁-C₃); G₃ (attached to X4)=H, lower CI-C₃ alkyl, phenyl or pyridyl rings which are optionally substituted with F, Cl, Br, CF₃, OR, methoxycarbonyl, carboxy, (CRR₁)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, tetrazolyl (Tzl), SO₂NRR1, alkylsulfonyl (C₁-C₃), CF₂P(═O)(OR)(OR1), phenyl (optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, tetrazolyl (Tzl), SO2NRR1, alkylsulfonyl (CI-C₃), CF₂P(═O)(OR)(OR1) , phenoxy (optionally further substituted with F, Cl, Br, CF₃, OR, methoxycarbonyl, carboxy, (CRR1),CO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, tetrazolyl (Tzl), SO₂NRR1, alkylsulfonyl (CI-C3), CF₂P(═O)(OR)(OR1)), benzyloxy (optionally further optionally substituted with F, Cl, Br, CF₃, OR, methoxycarbonyl, carboxy, (CRR1),CO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, tetrazolyl (Tzl), SO₂NRR1, alkylsulfonyl (CI-C₃), CF₂P(═O)(OR)(OR1)); and G₄ (attached to X₃)=H, F, Cl, alkyl (CI-C₃), methoxy, methylthio, dimethylamino, (CRR1),CO₂R, (CRR1)ₙCONRR1, (CRR1)ₙCONR(2-pyridyl, 2-imidazolyl, 2-thiazolyl), C(RRI)ₙNRR1, C(RR1)ₙNRC(═O)CR(RR1), C(RRI),NRC(═O)NR, C(RR1)nN(R)C(═O)OR1, C(RR1)n NRC(═O)C(═O)OR1, CF₂CO₂R, C(═O)NRR1. In some embodiments, if X₃=N, then there is no G₄ substituent.

Specific examples according to Formula I include compounds where: XI=C, X₂ N, X₃=C or N, X₄=C, X₅=N, L₁ =CH₂, L₂ =bond or CH₂, and L₃ =bond. Thus, specific examples of G₁ include 4-(difluoro-phosphono-methyl)-3-bromo-benzyl, (4-{4-[(diethoxy-phosphoryl)-difluoro-methyl]-3-bromo-benzylsulfanylmethyl, (4-{4-[(Ethoxy-phoshoryl)-difluoro-methyl]-3-bromo-benzylsulfanylmethyl, 3-bromo-4-carboxymethoxy-benzyl, 3-bromo-4-(2-carboxyvinyl)-benzyl, 4-(Carboxy-difluoromethyl)-benzyl, 4-{[(2,2-dimethyl-propionyloxymethoxy)-hydroxy-phosphoryl]-difluoro-methyl-benzyl, 4-(difluoro-phosphono-methyl-benzyl, and 4-carboxybenzyl.

Specific examples of G₂ include 3,4-dichlorophenyl, 4-methoxycarbonyl-benzyl, and 4-carboxybenzyl. Specific examples of G₃ include phenyl, 4-methoxycarbonylphenyl, 4-carboxyphenyl, 4-methylsulfonylphenyl, 4-(4′-methoxycarbonyl-phenoxy)-phenyl, 3-(3′-methoxycarbonyl-phenoxy)-phenyl, 3-(2′-methoxycarbonyl-phenoxy)-phenyl, 4-(2′-methoxycarbonyl-phenyl)-phenyl, 4-(4′-carboxyphenoxy)phenyl, 3-(3′-carboxyphenoxy)phenyl, 3-(2′-carboxyphenoxy)phenyl, 3-(methoxycarbonyl-phenyl-methoxy)-phenyl, 3-(carboxy-phenyl-methoxy)- phenyl, 3-phenoxy-phenyl. A specific example of G₄ is no substituent.

### d. Provisos Related to Formula I

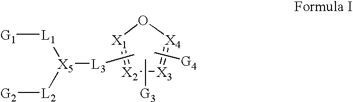 The following structures are excluded from Formula I as shown above, which might otherwise fall within the scope of Formula I:
(A) Any thioether, sulfoxide or sulfone in which G₁-L₁, G₂-L₂, G₁-L₁-X₅, G₁-L₁-X₅-L₃, G₃ and/or G₄ from Formula I (above) can be represented by Substructure II (below) linked to a carbon atom:

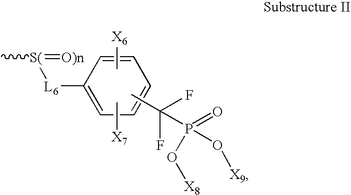

where n=0, 1 or 2, L₆ is a bond, a C1-6 alkylene group, or a C2-C6 alkenylene group, optionally substituted with one or more substituents chosen from (a) 1-12 halogen atoms and (b) OH, Oalkyl (C1-C4), in which the C1-C4 alkyl is optionally substituted with 1-9 halogen atoms, acyloxy groups, or alkoxycarbonyloxy groups;

X₆ and X₇ are each independently selected from the group: H, OH, halogen, CN, COOH, COOalkyl (C1-6), alkyl (C1-6), alkenyl (C2-6), alkynyl (C2-6), O-alkyl (C1-6), O-alkenyl (C2-6), C(═O)alkyl (C1-6), C(═O)alkenyl (C2-6), OC(═O)alkyl (C 1-6), OC(═O)alkenyl (C2-6), S(═O)ₓalkyl (C1-6, S(═O)ₓalkenyl (C2-6), SO2NY₁Y₂, C(═O)N Y₁Y₂, and N Y₁Y₂, where each alkyl, alkenyl, or alkynyl group in each substituent may optionally be substituted with one or more substituents independently selected from the following groups of substituents: (a) 1-13 halogen atoms, and (b) 1-2 substituents independently selected from Oalkyl (C1-3), C(═O)alkyl (C1-3), OC(═O)alkyl(C1-3), COOH, and C(═O)Oalkyl (C1-3);

Y₁ and Y₂ are each independently chosen from the following: H, alkyl (C1-4), where the alkyl groups are optionally substituted with 1-9 halogen atoms;

where each x is independently 0, 1 or 2;

and where X₈ and X₉ are each independently H, alkyl, aryl, or any other group attached through carbon, and pharmaceutically acceptable salts thereof.
(B) Formula I, in which G₁-L₁-X₅-L₃, G₃ and/or G₄ can be represented by Substructure III (below) linked to a carbon atom in the 5-membered heterocyclic ring:

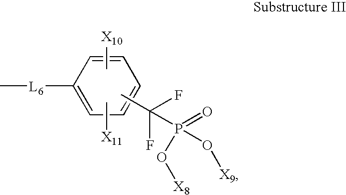

where L₆ is CH₂CH₂, optionally and independently substituted with COOY₃, aryl, alkyl, arylalkyl, S Y₃, SO Y₃, SO₂ Y₃, or any other group that can be linked by a single bond;

X₈ and X₉ are each independently H, alkyl, aryl, or any other group attached through carbon, and pharmaceutically acceptable salts thereof;

X₁₀ and X₁₁, are each independently selected from the group: H, OH, halogen, CN, COOH, COOalkyl (C1-6), alkyl (C1-6), alkenyl (C2-6), alkynyl (C2-6), O-alkyl (C1-6), O-alkenyl (C2-6)., C(═O)alkyl (C1-6), C(═O)alkenyl (C2-6)., OC(═O)alkyl (C1-6), OC(═O)alkenyl (C2-6)., S(═O)ₓalkyl (C1-6, S(═O)ₓalkenyl (C2-6), SO₂NY₄Y₅, C(═O)N Y₄Y₅, and N Y₄Y₅, where each alkyl, alkenyl, or alkynyl group in each substituent may optionally be substituted with one or more substituents independently selected from the following groups of substituents: (a) 1-13 halogen atoms, and (b) 1-2 substituents independently selected from Oalkyl (C1-3), C(═O)alkyl (C1-3), OC(═O)alkyl(C1-3), COOH, and C(═O)Oalkyl (C1-3);

Y₄ and Y₅ are each independently chosen from the following: H, alkyl (C1-4), where the alkyl groups are optionally and independently substituted with 1-9 halogen atoms, acyloxy groups, or alkyoxycarbonyloxy groups;

and where each x is independently 0, 1 or 2.

(C) Formula I, in which L₁ is (CH₂)ₙ, X₅ =N, L₂ =(CH₂)ₘ where n=0-2, m=0-2, optionally and independently substituted with COOY₅, aryl, alkyl, arylalkyl, SY₅, SOY₅, SO₂Y₅, or any other group that can be linked by a single bond;

G₂={CH(Z₁)(Z₂)}], where Z₁ and Z₂ are each independently any non-hydrogen substituent linked through a single bond, and in which G₁ is represented by Substructure IV:

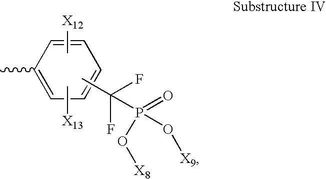

where X₈ and X₉ are each independently H, alkyl, aryl, or any other group attached through carbon, and pharmaceutically acceptable salts thereof;

where X₁₂ and X₁₃ are each independently selected from this group: H, OH, halogen, CN, COOH, COOalkyl (C1-6), alkyl (C1-6), alkenyl (C2-6), alkynyl (C2-6), O-alkyl (C1-6), O-alkenyl (C2-6), C(═O)alkyl (C1-6), C(═O)alkenyl (C2-6), OC(═O)alkyl (C1-6), OC(═O)alkenyl (C2-6), S(═O)ₓalkyl (C1-6, S(═O)ₓalkenyl (C2-6), SO₂NY₆Y₇, C(═O)N Y₆Y₇, and N Y₆Y₇, where each alkyl, alkenyl, or alkynyl group in each substituent may optionally be substituted with one or more substituents independently selected from the following groups of substituents: (a) 1-13 halogen atoms, and (b) 1-2 substituents independently selected from Oalkyl (C1-3), C(═O)alkyl (C1-3), OC(═O)alkyl(C1-3), COOH, and C(═O)Oalkyl (C1-3); and

where Y₆ and Y₇ are each independently chosen from the following: H, alkyl (C1-4), where the alkyl groups are optionally and independently substituted with 1-9 halogen atoms, acyloxy groups, or alkyoxycarbonyloxy groups, and where each x is independently 0, 1 or 2. and G₃=

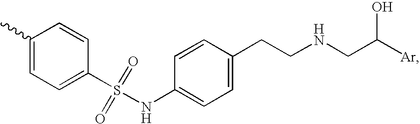

where Ar is any aryl or heteroaryl group as previously defined, optionally substituted with 1-3 substituents.

(E) Formula I, in which L₁=(CH2)ₘ; L₂ =bond; L₃ =bond, CO, or CONH; X₁=C, X₂=N, X₃=C, X₄=C, and X₅=CH or N, G₁=phenyl substituted with CO₂R7, X₁₃—A₁—CO₂R₇ [in which X₁₃ =O, NH, lower alkylamino, S(═O)_{q}, A₁=(CH2)ₘNH, (CH₂)ₘCONH, (CH₂)ₘCO, or (CH₂)ₘ, and R7=H or lower alkyl], or tetrazolyl, and optionally further substituted with one or more of the following: halogen, OH, lower cycloalkylalkyloxy, aralkyloxy, cyano, NO₂, lower alkoxy, or lower haloalkoxy; G₂=H; G₃ (attached to X₄)=an aromatic or heteroaromatic ring substituted with Y₈-A₂-Y₉ [where Y=O, S(═O)_{q}, NH, NHCO, NHSO₂, SO₂NH, CH₂, CO and A₂=lower alkylene and Y₉=lower cycloalkyl optionally substituted by phenyl, wherein Y₈, A₂, and Y₉ are optionally and independently further substituted with lower cycloalkyl, aromatic, heteroaromatic, piperazinyl, or indanyl], and optionally further substituted with halogen, lower alkyl, lower alkoxy, cyano, NO₂, lower cycloalkyl, S(═O)_{q}R₁₇ (wherein R₁₇=lower alkyl or aryl); G₄ (attached to X₃)=H, and where m=0 to 8, n=0 to 3, and q=0-2.

(F) Formula I, in which the oxa/aza heteroaromatic ring X₁X₂X₃X₄O is fused to another aromatic or heteroaromatic ring.

2. Formula V

In another aspect, compounds having the formula: 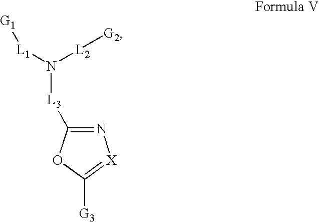
or pharmaceutically acceptable salts thereof, are provided. Compounds according to Formula V can be used to inhibit tyrosine phosphatase activity, e.g., PTP-1B activity, and thus find use in the treatment of various diseases such as obesity, diabetes, cancer, and neurodegenerative diseases.

In Formula V, L₁, L₂, and L₃ can be, independently, a bond or (CH₂)ₛ where s is 1-3, in one embodiment s is 1;
X is CR7 or N, where R7 is H or C1-C3 alkyl;
G₁ is H or a phenyl ring, where the phenyl ring is optionally substituted with one or more moieties selected from the group consisting of: phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester, where acyl is C₂-C₇ alkanoyl or C4-C7 cycloalkanoyl, phosphonodifluoromethyl mono-alkoxyalkyl ester, where alkoxy is CI 5-C22, phosphonodifluoromethyl mono-alkoxycarbonyloxymethyl ester, where alkoxy is C1-C6 or C3-C6 cycloalkoxy, 2-carboxyethenyl optionally substituted with 1-2 fluorines or methyl groups, carboxymethoxy, carboxy-C2-C4-alkyl optionally fuirther substituted with 1-4 halogen atoms or 1-4 methyl groups, Cl, Br, F, CN, OH, CH₃, and ethynyl;
G₂ is H, C₁-C₃ alkyl, or a phenyl or pyridyl ring, where the phenyl or pyridyl ring is optionally and independently substituted with 1, 2, or 3 of the following moieties: Cl, F, Br, carboxy, methoxycarbonyl, OCF₃, OCHF₂, C₁-C₃ alkyl, and C₁-C₃-alkylsulfonyl;
G₃ is H, C₁-C₃ alkyl, or a phenyl or pyridyl ring, where the phenyl or pyridyl ring is optionally substituted with:

(i) F, Cl, Br, CF₃, OR, methoxycarbonyl, carboxy, (CRR₁)ₙCO₂R, CF₂CO₂R, O(CRR₁)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR₁)CO₂R, SO₂NRR1, C₁-C₃-alkylsulfonyl, or CF₂P(═O)(OR)(OR1);

(ii) phenyl, where the phenyl is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1);

(iii) phenoxy, where the phenoxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1); and

(iv) benzyloxy, where the benzyloxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, SO₂NRR1, C₁-C₃-alkylsulfonyl, or CF₂P(═O)(OR)(OR1),
where m=0 to 6 and n=0 to 2; and

where R and R1 are independently selected from hydrogen, an alkyl group of 1 to 6 carbon atoms, where the alkyl group is unsubstituted or mono-, di- or tri-substituted with 1 to 3 substituents selected from the group consisting of Y₁, Y₂, Y₃, an aryl group, —OC(R2R3)OC(═O)R4, and —OC(R2R3)OC(═O)OR4, or where R and R1 are joined to form a 4-8 membered cycloalkyl, cycloalkenyl, cycloalkynyl, or heterocyclic ring;

where R2, R3 and R4 are independently selected from (i) and (ii) as follows:

(i) H, C₁-C₇ alkyl, alkenyl of 2 to 6 carbon atoms, where the alkenyl group is unsubstituted or mono-, di- or tri-substituted with 1 to 3 substituents selected from the group consisting of Y₁, Y₂, and Y₃, alkynyl of 2 to 6 carbon atoms, where the alkynyl group is unsubstituted or mono-, di- or tri-substituted with 1 to 3 substituents selected from the group consisting of Y₁, Y₂, and Y₃, cycloalkyl of 3 to 8 carbon atoms, where the cycloalkyl group is unsubstituted or mono-, di- or tri-substituted with 1 to 3 substituents selected from the group consisting of Y₁, Y₂, and Y₃, aryl of 6 to 14 carbon atoms, where the aryl group is unsubstituted or mono-, di- or tri-substituted with 1 to 3 substituents selected from the group consisting of Y₁, Y₂, and Y₃, linked biaryl or heterobiaryl groups of 10 to 20 atoms featuring two aromatic or heteroaromatic ring systems linked through a single bond, with the ring atoms selected from carbon and heteroatoms, where the heteroatoms are selected from oxygen, nitrogen, and sulfur, and where the linked biaryl or heterobiaryl group is unsubstituted or mono-, di- or tri-substituted with 1 to 3 substituents selected from the group consisting of Y₁, Y₂, and Y₃, aralkyl of 7 to 16 carbon atoms, where the aralkyl is unsubstituted or mono-, di- or tri-substituted with 1 to 3 substituents selected from the group consisting of Y₁, Y₂, and Y₃, monocyclic-heteroaryl or bicyclic-heteroaryl having 5 to 14 ring atoms with the ring atoms selected from carbon and heteroatoms, where the heteroatoms are selected from oxygen, nitrogen, and sulfur, and where the monocyclic-heteroaryl or bicyclic heteroaryl group is unsubstituted or mono-, di- or tri-substituted with 1 to 3 substituents selected from the group consisting of Y₁, Y₂, and Y₃, and a heteroaralkyl group of 5 to 14 ring atoms with the ring atoms selected from carbon and heteroatoms, where the heteroatoms are selected from oxygen, nitrogen, and sulfur, where the heteroaralkyl is unsubstituted or substituted on the alkyl chain and which is unsubstituted on the ring or mono-, di- or tri-substituted on the ring with 1 to 3 substituents selected from the group consisting of Y₁, Y₂, and Y₃; or

(ii) R2 and R3, and/or R3 and R4, and/or R2 and R4 are joined to form a 4-8-membered cycloalkyl, cycloalkenyl, cycloalkynyl, or heterocyclic ring, and the other of R2, R3, and R4, when not joined in a ring, is selected as in (i) above;

and wherein Y1, Y2, and Y3 are independently selected from (i) or (ii) as follows:

(i) R5, (CR5R6)ₙOR5, OH, (CR5R6)ₙNR5R6, C(═NR5)NR5R6, C(═NOR5)NR5R6, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5, C(═O)R5,—(CR5R6)ₙC(═O)R5, (CF₂)ₙC(═O)R5, (CFR5) nC(═O)R5, tetrazolyl (Tzl), (CR5R6)ₙTzl, (CF₂)ₙTzl, (CFR5)ₙTzl, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, (CR5R6)ₙC(═O)C(═O)OR5, (CR5R6)ₙCH(OR5)C(═O)OR5, C(R5)═C(R6), C(═O)OR5, C(R5)═C(R6)-Tzl, (CR5R6)ₙP(═O)(OH)₂, (CR5R6)ₙP(═O)(OR5)(OR6), P(═O)(OR5)[(OCR5R6)OC(═O)R5], P(═O)(OR5)[(OCR5R6)OC(═O)OR5], P(═O)[(OCR5R6)OC(═O)R5)][(OCR5R6)OC(═O)R5], P(═O)[(OCR5R6)OC(═O)OR5)][(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5═CR5-P(═O)(OR5)(OR6), CR5═CR5-P(═O)(Me)(OR5), CC-P(═O)(OR5)(OR6), (C═O)P(═O)(OR5)(OR6), (C═O)P(═O)(Me)(OR5), (C═O)P(═O)(CF₃)(OR5), (CR5OR6)ₙP(═O)(OR5)(OR6), (CR5OR6)ₙP(═O)(Me)(OR5), (CR5OR6)ₙP(═O)(CF₃)(OR5), O(CR5R6)ₙC(═O)OR5, O(CF₂)ₙC(═O)OR5, OCH[C(═O)OR5]₂, O(CR5R6)ₙCH[C(═O)OR5]₂, OCF[C(═O)OR5]₂, O(CR5R6)ₙC(═O)C(═O)OR5, O(CF₂)ₙC(═O)C(═O)OR5, O(CR5R6)ₙTzl, O(CF₂)ₙTzl, OCH(Tzl)₂, O(CF₂)ₙP(═O)(OR5)(OR6), O(CF₂)ₙP(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF[P(═O)(Me)(OR5)]₂, SO₃H, —(CR5R6)ₙSO₃H, S(O)ₙR5, SCF₃, SCHF₂, SO₂CF₃, SO₂Ph, (CR5R6)ₙS(O)ₙR5, (CR5R6)ₙS(O)₂CF₃, (CR5R6)ₙSO₂NR5R6, (CR5R6)ₙSO₂NR5C(═O)(Me, CF₃), (CF₂)ₙSO₃H, (CFR5)ₙSO₃H, and (CF₂)ₙSO₂NR5R6, where n=0-2, and where R5 and R6 are each independently H, C1-C6 alkyl, C2-C6 alkenyl, C2-C6 alkynyl, a C3-C8 cycloalkyl ring, or a 5-7 membered heterocyclic ring; or

(ii) Y₁ and Y₂, and/or Y₁ and Y₃, and/or Y₂ and Y₃ are selected together to be (CR5R6)₂₋₆, —O[C(R8)(R9)]ᵣO— or —O[C(R8)(R9)]ᵣ₊₁—, where r is an integer from 1 to 4 and R8 and R9 are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroarylalkyl of 5 to 14 ring atoms, and the other of Y1, Y2, and Y3, when not selected as in (ii), is selected as in (i) above.

In certain embodiments of compounds according to Formula V, X is CR7, and R7 is H. In other instances, L₁ is CH₂ and/or L₃ is a bond. G₁ can be an optionally substituted phenyl ring, such as a phenyl ring substituted at the 3 position, the 4 position, or at both the 3 and 4 positions. For example, G₁ can be a phenyl ring substituted with one or more of the following moieties: phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester, where acyl is C₂-C₇ alkanoyl or C4-C7 cycloalkanoyl, phosphonodifluoromethyl mono-alkoxycarbonyloxymethyl ester, where alkoxy is C1-C6 or C3-C6 cycloalkoxy, 2-carboxyethenyl, carboxymethoxy, carboxy-C2-C4-alkyl, Cl, Br, and F. In certain embodiments, G₁ is selected from the group consisting of: 4-(difluoro-phosphono-methyl)-3-bromo-benzyl, (4-{4-[(diethoxy-phosphoryl)-difluoro-methyl]-3-bromo-phenyl, 3-bromo-4-carboxymethoxy-benzyl, (4-{4-[(ethoxy-hydroxy-phosphoryl)-difluoro-methyl]-3-bromo-benzyl, 3-bromo-4-(2-carboxyvinyl)-benzyl, 4-(carboxy-difluoro-methyl)-benzyl, 4-{[(2,2-dimethyl-propionyloxymethoxy)-hydroxy-phosphoryl]-difluoro-methyl-3-bromo-benzyl, 4-(difluoro-(methoxy-hydroxy-phosphoryl)-methyl-3-bromo-benzyl, 4-(difluoro-phosphono-methyl)-benzyl, 4-carboxybenzyl, 4-(difluoro-(3-hexadecyloxy-propoxy)-hydroxy-phosphoryl)methyl)-3-bromobenzyl, 4-(difluoro-phosphono-methyl)-3-chloro-benzyl, and 4-(difluoro-((1-isopropoxycarbonyloxy)ethoxy-hydroxy-phosphoryl)methyl)-3-bromo-benzyl. In other embodiments, G₁ is selected from the group consisting of: 4-(difluoro-phosphono-methyl)-3-bromo-phenyl, (4-{4-[(diethoxy-phosphoryl)-difluoro-methyl]-3-bromo-phenyl, 3-bromo-4-carboxymethoxy-phenyl, (4-{4-[(ethoxy-phosphoryl)-difluoro-methyl]-3-bromo-phenyl, 3-bromo-4-(2-carboxyvinyl)-phenyl, 4-(carboxy-difluoro-methyl)-phenyl, 4-{[(2,2-dimethyl-propionyloxymethoxy)-hydroxy-phosphoryl]-difluoro-methyl-3-bromo-phenyl, 4-(difluoro-(methoxy-hydroxy-phosphoryl)-methyl-3-bromo-phenyl, 4-(difluoro-phosphono-methyl)-phenyl, 4-carboxyphenyl, 4-(difluoro-(3-hexadecyloxy-propoxy)-hydroxy-phosphoryl)methyl)-3-bromobenzyl, 4-(difluoro-phosphono-methyl)-3-chloro-phenyl and 4-(difluoro-((1-isopropoxycarbonyloxy)ethoxy-hydroxy-phosphoryl)methyl)-3-bromo-phenyl. In some cases, G₁ is a phenyl ring substituted with phosphonodifluoromethyl.

In certain embodiments, G₂ is a phenyl ring substituted at the 3 position, the 4 position, or at both the 3 and 4 positions. In one embodiment, G2 is selected from the group consisting of methyl, phenyl, 4-fluorophenyl, 4-chlorophenyl, 3,4-dichlorophenyl, 4-methoxycarbonyl-benzyl, and 4-carboxybenzyl.

In some embodiments, G₃ can be a substituted phenyl ring, e.g., a phenyl ring substituted at the 3 position, the 4 position, or at both the 3 and 4 positions. For example, G₃ is selected from the group consisting of phenyl, 4-methoxycarbonylphenyl, 4-carboxyphenyl, 4-aminocarbonylphenyl, 4-methylsulfonylphenyl, 4-(4′-methoxycarbonyl-phenoxy)-phenyl, 4-(4′-carboxyphenoxy)phenyl, 3-((α-methoxycarbonylbenzyloxy)phenyl, 3-(2′-methoxycarbonyl-phenoxy)-phenyl, 4-(2′-methoxycarbonyl-phenyl)-phenyl, 3-(α-carboxybenzyloxy)phenyl, 3-(2′-carboxyphenoxy)phenyl, 3-(2′-methoxycarbonyl-phenyl)-phenyl, 3-(2′-carboxy-phenyl)-phenyl, 3-phenoxy-phenyl, 3-(3′-carboxyphenoxy)phenyl, 3-(3′-methoxycarbonylphenoxy)phenyl, and 4-(2 ′-carboxyphenyl)phenyl.

3. Pro-Drug Compounds

A compound can be modified to act as a prodrug. It is a well-known phenomenon in drug discovery that compounds such as enzyme inhibitors can display potency and selectivity in in vitro assays, yet not readily manifest the same activity in vivo. This lack of “bioavailability” may be due to a number of factors, such as poor absorption in the gut, first-pass metabolism in the liver, and poor uptake in the cells. Although the factors determining bioavailability are not completely understood, there are many techniques known by those skilled in the art to modify compounds, which are potent and selective in biochemical assays but show low or no activity in vivo, into drugs that are biologically and therapeutically active.

It is considered to be within the scope of the present disclosure to modify any of the compounds provided herein (termed the ‘original compound’) by attaching chemical groups that will improve the bioavailability of the original compound. Examples of said modifications include changing of one or more carboxy groups to esters (for instance methyl esters, ethyl esters, acetoxymethyl esters or other acyloxy-methyl esters). Compounds provided herein so modified by attaching chemical groups are termed ‘modified compounds.’

Other examples of modified compounds are compounds that have been cyclized at specific positions (‘cyclic compounds’) which upon uptake in cells or mammals become hydrolyzed at the same specific position(s) in the molecule to yield the compounds provided herein, the original compounds, which are then said to be ‘non-cyclic’. For the avoidance of doubt, it is understood that the latter original compounds in most cases will contain other cyclic or heterocyclic structures that will not be hydrolyzed after uptake in cells or mammals.

Generally, said modified compounds will not show a behavior in biochemical assays similar to that of the original compound, i.e., the corresponding compounds provided herein without the attached chemical groups or said modifications. Said modified compounds may even be inactive in biochemical assays. However, after uptake in cells or mammals these attached chemical groups of the modified compounds may in turn be removed spontaneously or by endogenous enzymes or enzyme systems to yield compounds provided herein, original compounds. ‘Uptake’ is defined as any process that will lead to a substantial concentration of the compound inside cells or in mammals. After uptake in cells or mammals and after removal of said attached chemical group or hydrolysis of said cyclic compound, the compounds may have the same structure as the original compounds and thereby regain their activity and hence become active in cells and/or in vivo after uptake.

A number of techniques well known to those skilled in the art may be used to verify that the attached chemical groups have been removed or that the cyclic compound has been hydrolyzed after uptake in cells or mammals. One example of such techniques is as follows: A mammalian cell line, which can be obtained from the American Type Culture Collection (ATCC) or other similar governmental or commercial sources, is incubated with a modified compound. After incubation under appropriate conditions, the cells are washed, lysed and the lysate is isolated. A number of different procedures, well known to those skilled in the art, may in turn be used to extract and purify the modified compound (or a metabolite thereof) (the ‘purified compound’) from the lysate. The modified compound may or may not retain the attached chemical group or the cyclic compound may or may not have been hydrolyzed. Similarly, a number of different procedures may be used to structurally and chemically characterize the purified compound. Since the purified compound has been isolated from said cell lysate and hence has been taken up by said cell line, a comparison of the structurally and chemically characterized compound with that of the original compound (i.e. without the attached chemical group or other modification) will provide information on whether the attached chemical group as been removed in the cell or if the cyclic compound has been hydrolyzed.

As a further analysis, the purified compound may be subjected to enzyme kinetic analysis as described in detail in the present description. If the kinetic profile is similar to that of the original compound without the attached chemical group, but different from the modified compound, this result confirms that the chemical group has been removed or the cyclic compounds has been hydrolyzed. Similar techniques may be used to analyze compounds provided herein in whole animals and mammals.

One form of prodrug is to prepare acetoxymethyl esters of the compounds provided herein, which may be prepared by the general procedure reported by C. Schultz et al., J. Biol. Chem. 1993, 268:6316-6322:

A carboxylic acid (1 eq) is suspended in dry acetonitrile (2 mL/0.1 mmol). Diisopropyl amine (3.0 eq) is added followed by bromomethyl acetate (1.5 eq). The mixture is stirred under nitrogen overnight at room temperature. Acetonitrile is removed under reduced pressure to yield an oil, which is diluted in ethylacetate and washed with water (3×). The organic layer is dried over anhydrous magnesium sulfate. Filtration, followed by solvent removal under reduced pressure, affords a crude oil. The product is purified by column chromatography on silica gel, using an appropriate solvent system.

Other prodrugs can routinely be prepared from compounds provided herein by the procedures outlined in the following reports: Stankovic, et al., “The Role of 4-Phosphonodifluoromethyl- and 4-Phosphono-phenylalanine in the Selectivity and Cellular Uptake of SH2 Domain Ligands.” Bioorg. Med. Chem. Lett. 1997; 7(14):1909-14; Ortmann R et al., “Acyloxyalkyl ester prodrugs of FR900098 with improved in vivo anti-malarial activity.” Bioorg. Med. Chem. Lett. 2003; 13(13):2163-6; Hughes W T et al., “Single-dose pharnacokinetics and safety of the oral antiviral compound adefovir dipivoxil in children infected with human immunodeficiency virus type 1.” Antimicrob Agents Chemother. 2000; 44(4):1041-6; Starrett J E Jr et al., “Synthesis and in vitro evaluation of a phosphonate prodrug: bis(pivaloyloxymethyl) 9-(2-phosphonylmethoxyethyl)adenine.” Antiviral Res. 1992; 19(3):267-73.

Such prodrug preparations are routinely prepared, once a novel drug compound is identified, such as the novel PTP-1B inhibitors disclosed herein.

Other prodrugs of the compounds provided herein are prodrugs of difluoromethylphosphonic acids and have the formulae ArCF₂P(O)(OH)(OCH(H/Me)OC(═O)OiPr, ArCF₂P(O)[(OCH(H/Me)OC(═O)OiPr]₂, ArCF₂P(O)(OH)(OCH(H/Me)OC(═O)tBu, or ArCF₂P(O)[(OCH(H/Me)OC(═O)tBu]₂. Other prodrugs of the compounds provided herein have the formulae ROCH₂CHR′CH₂O-P(O)(OH)CF₂Ar or (ROCH₂CHR′CH₂O)₂-P(O)CF₂Ar, where R is C₁₄₋₂₀-n-alkyl and R′ is H, OH or OMe. Further prodrugs of the compounds provided herein are prodrugs as described in EP 0 350 287; EP 0 674 646; U.S. Pat. No. 6,599,887; U.S. Pat. No. 6,448,392; U.S. Pat. No. 6,752,981; U.S. Pat. No. 6,312,662; U.S. 2002/0173490; Friis et al. Eur. J Pharm. Sci. 4:49-59 (1996); Erion et al. J. Am. Chem. Soc. 126:5154-5163 (2004); WO 03/095665; Krise et al. Adv. Drug. Deliv. Rev. 19:287-310 (1996); and Ettmayer et al. J. Med. Chem. 47:2393-2404 (2004). The disclosures of these patents and publications are incorporated by reference herein in their entirety.

Examples of these prodrugs are shown in the table below.

| Structure | Example | Chemical Name |
|---|---|---|
| 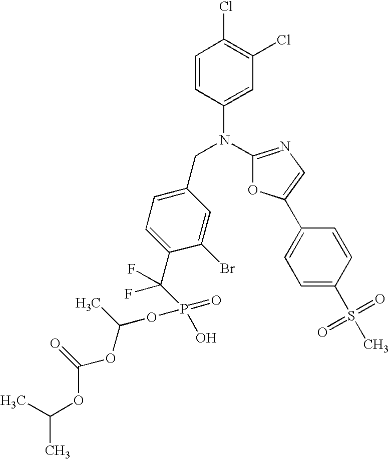 | 33 | {[2-Bromo-4-({(3,4-dichloro-phenyl)-[5-(4- methanesulfonyl-phenyl)-oxazol-2-yl]- amino}-methyl)-phenyl]-difluoro-methyl}- phosphonic acid mono-(1- isopropoxycarbonyloxy-ethyl) ester |
| 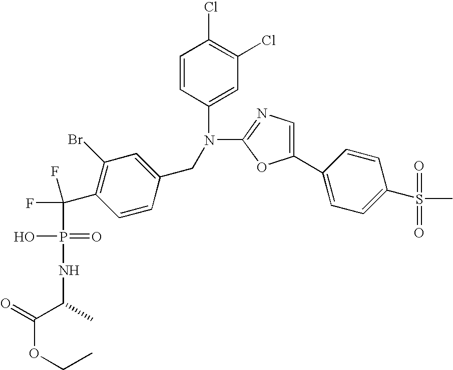 | 34 | 2-({[2-Bromo-4-({(3,4-dichloro-phenyl)-[5- (4-methanesulfonyl-phenyl)-oxazol-2-yl]- amino}-methyl)-phenyl]-difluoro-methyl}- hydroxy-phosphinoylamino)-propionic acid ethyl ester |
| 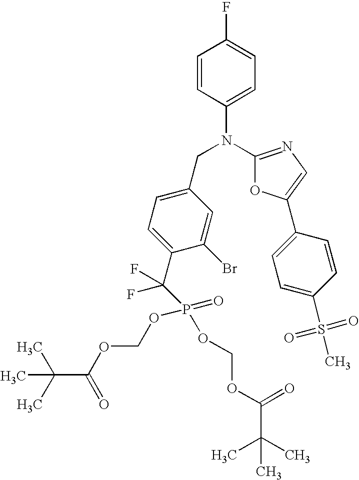 | 35 | 2,2-Dimethyl-propionic acid {[2-bromo-4- ({(4-fluoro-phenyl)-[5-(4-methanesulfonyl- phenyl)-oxazol-2-yl]-amino}-methyl)- phenyl]-difluoro-methyl-(2,2-dimethyl- propionyloxymethoxy)- phosphinoyloxymethyl ester |

4. Methods

The compounds provided herein inhibit tyrosine phosphatases, including PTP-1B, and thus improve insulin sensitivity, among other benefits. The compounds therefore will find use in preventing, treating, or ameliorating one or more symptoms associated with Type 1 and Type 2 diabetes (and associated complications such as hypertension, ischemic diseases of the large and small blood vessels, blindness, circulatory problems, kidney failure and atherosclerosis), syndrome X, metabolic syndrome, improving glucose tolerance, improving insulin sensitivity when there is insulin resistance, improving leptin sensitivity where there is leptin resistance, lowering body weight, and preventing or treating obesity. In addition, the compounds will be useful in preventing, treating, or ameliorating one or more of the symptoms associated with cancer, neurodegenerative diseases, and the like.

C. Procedures for the Synthesis of Compounds and Intermediates

Details concerning the preparation of these compounds are provided in the following experimental section. Other compounds within the scope of this disclosure may be prepared by minor modification of the syntheses shown herein using readily available starting materials. For a range of reaction conditions, reagents, solvents, catalysts and conditions that would be useful for preparing depicted intermediates and final targets, the following authoritative works are cited: R. C. Larock, Comprehensive Organic Transformations, 2^{nd} ed., Wiley-VCH, New York, N.Y., 1999; Comprehensive Organic Chemistry, ed. D. H. R. Barton and W. D. Ollis, Pergamon Press, Oxford, 1979; Comprehensive Organic Synthesis, ed. B. M. Trost and I. Fleming, Pergamon Press, Oxford, 1991. For relevant comprehensive reviews and references on protection/deprotection strategies used herein, see: Protective Groups in Organic Synthesis, 3rd ed., ed. T. W. Greene and P. G. M. Wuts, John Wiley and Sons, New York, N.Y., 1999; cf. Ch. 5 (carboxyl), Ch. 3 (phenols), Ch. 7 (amino groups), Ch. 9 (phosphate). Representative syntheses are shown in the scheme below. 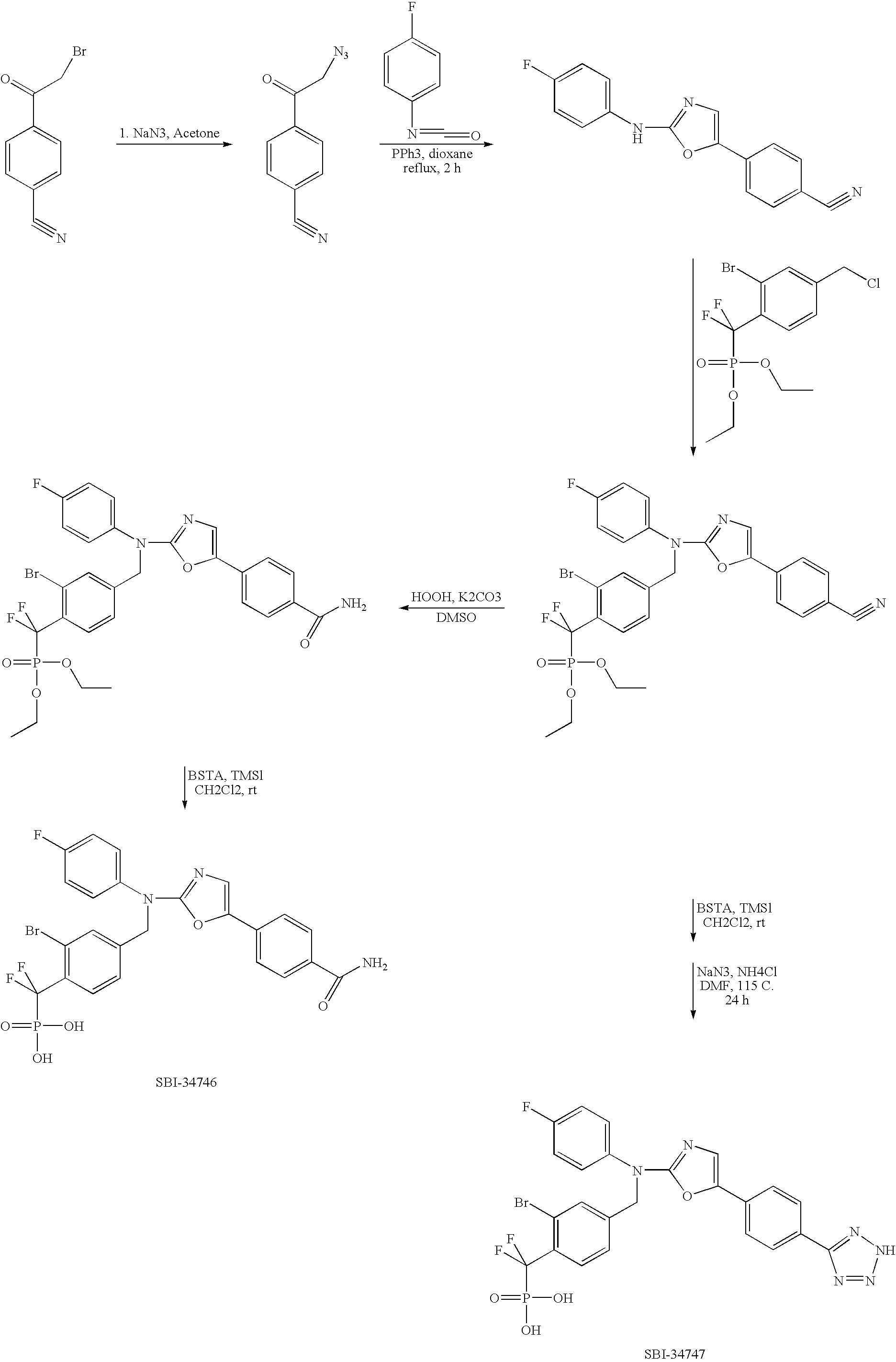
D. Methods for Treating, Preventing, or Ameliorating a Symptom of a Disease

The compounds described herein inhibit tyrosine phosphatases, including PTP-1B, and thus can improve insulin sensitivity, among other benefits. The compounds therefore can find use in preventing, treating, or ameliorating one or more symptoms of Type 1 and Type 2 diabetes, improving glucose tolerance, improving insulin sensitivity when there is insulin resistance, lowering body weight, and preventing or treating obesity. In addition, the compounds will be useful in preventing, treating, or ameliorating one or more of the symptoms of cancer, neurodegenerative diseases, and the like.

In any of the methods, a compound or pharmaceutical composition including a compound described herein can be administered to a mammal, e.g., a human. The compound or pharmaceutical composition can be administered in a therapeutically effective amount.

A pharmaceutical composition can include a compound described herein and a pharmaceutically acceptable carrier. As used herein, pharmaceutical composition and therapeutic preparation can be used interchangeably. For example, a compound can be provided together with physiologically tolerable (or pharmaceutically acceptable) liquid, gel or solid carriers, diluents, adjuvants and excipients. Such pharmaceutical compositions can be prepared as sprays (e.g. intranasal aerosols) for topical use. They may also be prepared either as liquid solutions or suspensions, or in solid forms including respirable and nonrespirable dry powders. Oral formulations (e.g. for gastrointestinal administration) usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. A pharmaceutical composition can take the form of a solution, suspension, tablet, pill, capsule, sustained release formulation, or powder, and typically contain 1%-95% of active ingredient (e.g., 2%-70%, 5%-50%, or 10-80%).

A compound can be mixed with diluents or excipients that are physiologically tolerable and compatible. Suitable diluents and excipients are, for example, water, saline, dextrose, glycerol, or the like, and combinations thereof. In addition, if desired, a composition may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

A pharmaceutical composition can be administered to a mammal (e.g., a human, mouse, rat, cat, monkey dog, horse, sheep, pig, or cow) at a therapeutically effective amount or dosage level. A therapeutically effective amount or dosage level of a compound can be a function of many variables, including the affinity of the inhibitor for the tyrosine phosphatase, any residual activity exhibited by competitive antagonists, the route of administration, the clinical condition of the patient, and whether the inhibitor is to be used for the prophylaxis or for the treatment of acute episodes.

Effective dosage levels can be determined experimentally, e.g., by initiating treatment at higher dosage levels and reducing the dosage level until relief from reaction is no longer obtained. Generally, therapeutic dosage levels will range from about 0.01-100 μg/kg of host body weight.

A compounds or pharmaceutical composition may also be administered in combination with one or more further pharmacologically active substances e.g., substances selected from antiobesity agents, antidiabetics, antihypertensive agents, agents for the treatment and/or prevention of complications resulting from or associated with diabetes, and agents for the treatment and/or prevention of complications and disorders resulting from or associated with obesity.

For example, a compound may be administered in combination with one or more antiobesity agents or appetite regulating agents. Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, B3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator activated receptor) modulators, RXR (retinoid X receptor) modulators and TR B agonists.

In one embodiment, the antiobesity agent is leptin. In other embodiments, the antiobesity agent is dexamphetamine or amphetamine, fenfluramine or dexfenfluramine, sibutramine, orlistat, mazindol or phentermine.

Suitable antidiabetics include insulin, GLP-1 (glucagons like peptide-1) derivatives such as those disclosed in WO 98/08871, which is incorporated herein by reference, as well as orally active hypoglycemic agents. Orally active hypoglycemic agents include sulphonylureas, biguanides, meglitinides, oxadiazolidinediones, thizolidinediones, glucosidase inhibitors, glucagons antagonists such as those disclosed in WO 99/01423, GLP-1 agonists, potassium channel openers such as those disclosed in WO 98/26265 and WO 99/03861, insulin sensitizers, DPP-IV (dipeptidyl peptidase-IV) inhibitors, inhibitors of hepatic enzymes involved in stimulation of gluconeogensis and/or glycogenolysis, glucose uptake modulators, compounds modifying the lipid metabolism such as antihyperlipidemic agents and antilipedimic agents as HMG CoA inhibitors (statins), compounds lowering food intake, PPAR and RXR agonists, and agents acting on the ATP-dependent potassium channel of the B-cells.

In another embodiment, a compound can be administered in combination with insulin. In other embodiments, a compound can be administered in combination with a sulphonylurea (e.g., tolbutamide, glibenclamide, glipizide or glicazide), a biguanide (e.g. metformin), a meglitinide (e.g., repaglinide), a thizolidinedione (e.g., troglitazone, ciglitazone, pioglitazone, rosiglitazone) or compounds disclosed in WO 97/41097 such as 5-[[4-[3-Methyl-4-oxo-3,4-dihydro-2-quinazolinyl]methoxy]phenyl-methyl]thiazolidine-2,4-dione, or a pharmaceutically acceptable salt of any of the foregoing, such as a potassium salt.

In an additional aspect, a compound may be administered in combination with an insulin sensitizer as disclosed in WO 99/19313, such as (−)3-[4-[2-Phenoxazin-10-yl)ethoxy]phenyl]-2-ethoxypropanoic acid or a pharmaceutically acceptable salt thereof, i.e. the arginine salt.

In further embodiments, a compound can be administered in combination with an a-glucosidase inhibitor (e.g. miglitol or acarbose), an agent acting on the ATP-dependent potassium channel of the B-cells (e.g. tolbutamide, glibenclamide, glipizide, glicazide or repaglinide), nateglinide, an antihyperlipidemic agent or antilipidemic agent (e.g., cholestyramine, colestipol, clofibrate, gemfibrozil, lovastatin, pravastatin, simvastatin, probucol or dextrothyroxine).

In other embodiments, a compound can be administered in combination with more than one of the above-mentioned compounds (e.g., in combination with a sulphonylurea and metformin, a sulphonylurea and acarbose, repaglinide and metformin, insulin and a sulphonylurea, insulin and metformin, insulin, insulin and lovastatin, etc.).

In another aspect, a compound can be administered in combination with one or more antihypertensive agents. Examples of antihypertensive agents are B-blockers such as alprenolol, atenolol, timolot, pindolol, propranolol and metoprolol, ACE (angiotensin converting enzyme) inhibitors such as benazepril, captopril, analapril, fosinopril, lisinopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of a compound with one or more of the above-mentioned agents and optionally one or more further pharmacologically active substances is considered to be within the scope of the present disclosure. When a compound is used with one or more other agents, in certain cases these other agents may be employed in lesser dosages than when used alone.

Where combinations are contemplated, it is not intended that the present disclosure be limited by the particular nature of the combination. The present discisoure contemplates combinations as simple mixtures as well as chemical hybrids. One example of the latter is where the present compound is covalently linked to a pharmaceutical compound, or where two or more compounds are joined. For example, covalent binding of the distinct chemical moieties can be accomplished by any one of many commercially available cross-linking compounds.

In view of the therapeutic urgency attendant acute episodes, a compound may be intravenously infused or introduced immediately upon the development of symptoms. Prophylaxis can be suitably accomplished, in certain cases, by intramuscular or subcutaneous administration. In this regard, the compositions can be prepared as injectables, either as liquid solutions or suspensions; solid forms suitable for solution in, or suspension in, liquid prior to injection may also be prepared.

E. Determination of Inhibitory Activity

The compounds provided herein are evaluated for biological activity as inhibitors of PTP-1B using, for example, a pNPP assay. Such an assay can be used to screen compounds for tyrosine phosphatase inhibitory activity as shown in Example 39.

Compounds which demonstrate inhibitory activity against tyrosine phosphatases can have application in the treatment of various diseases. For example, compounds which demonstrate inhibitory activity against PTP-1B can find use in the treatment of diabetes. Compounds which demonstrate such activity against CD45 can find use in the treatment of autoimmune diseases, inflammation, transplantation rejection reactions, and other diseases including arthritis, systemic lupus, Crohn's disease, inflammatory bowel disease, and other autoimmune disorders known to those skilled in the art. Compounds which demonstrate such activity against TC-PTP can find use in the treatment of cancer, typically as antiangiogenic agents.

In the case of compounds which demonstrate inhibitory activity against PTP-1B, one can test the compounds for blood glucose lowering effects in diabetic obese female ob/ob mice as follows: The mice will be of similar age and body weights and randomized into groups of ten mice. They have free access to food and water during the experiment. 5 The compounds are administered by either gavage, subcutaneous, intravenous or intraperitoneal injections. Examples of typical dose ranges for such evaluations are 0.1, 0.3, 1.0, 3.0, 10, 30, 100 mg per kg body weight. The blood glucose levels are measured twice before administration of the compounds provided herein. After administration of the compound, the blood glucose levels are measured at the following time points: 1, 2, 4, 6, and 8 hours. A positive response is defined either as (i) a more than 25 percent reduction in blood glucose levels in the group receiving the compound provided herein compared to the group receiving the vehicle at any time point or (ii) statistically significant (i.e., p<0.05) reduction in the area under the blood glucose curve during the whole period (i.e. 8 hrs) in the group treated with the compounds provided herein compared to controls. Compounds that show positive response can be used as development candidates for treatment of human diseases such as diabetes and obesity.

The following detailed examples are provided for illustration and are not to be considered as limiting the scope of the present disclosure. The structures of various of the 20 disclosed compounds will be found depicted in Table 1 and Table 2, below.

TABLE 1

| Structure | Example | Chemical Name | IC50 |
|---|---|---|---|
| 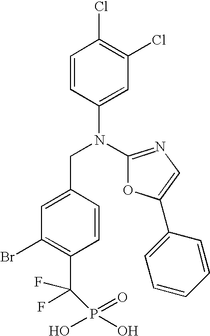 | 1 | [(2-Bromo-4-{[(3,4-dichloro- phenyl)-(5-phenyl-oxazol-2-yl)- amino]-methyl}-phenyl)- difluoro-methyl]-phosphonic acid | ++ |
| 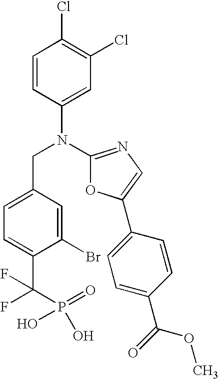 | 2 | 4-{2-[[3-Bromo-4-(difluoro- phosphono-methyl)-benzyl]-(3,4- dichloro-phenyl)-amino]-oxazol- 5-yl}-benzoic acid methyl ester | +++ |
| 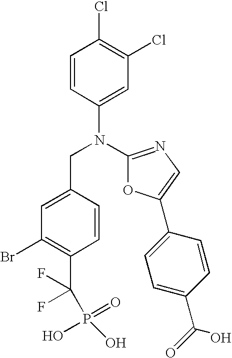 | 3 | 4-{2-[[3-Bromo-4-(difluoro- phosphono-methyl)-benzyl]-(3,4- dichloro-phenyl)-amino]-oxazol- 5-yl}-benzoic acid | +++++ |
| 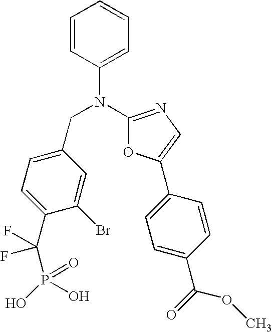 | 4 | 4-(2-{[3-Bromo-4-(difluoro- phosphono-methyl)-benzyl]- phenyl-amino}-oxazol-5-yl)- benzoic acid methyl ester | ++ |
| 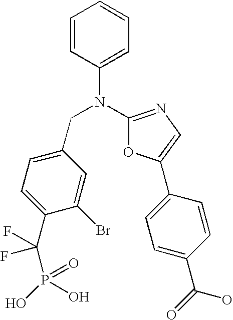 | 5 | 4-(2-{[3-Bromo-4-(difluoro- phosphono-methyl)-benzyl]- phenyl-amino}-oxazol-5-yl)- benzoic acid | ++++ |
| 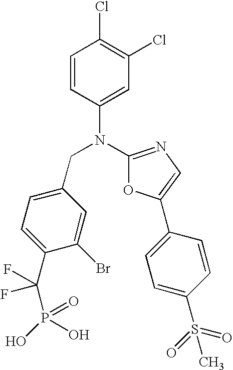 | 6 | {[2-Bromo-4-({(3,4-dichloro- phenyl)-[5-(4-methanesulfonyl- phenyl)-oxazol-2-yl]-amino}- methyl)-phenyl]-difluoro- methyl}-phosphonic acid | +++++ |
| 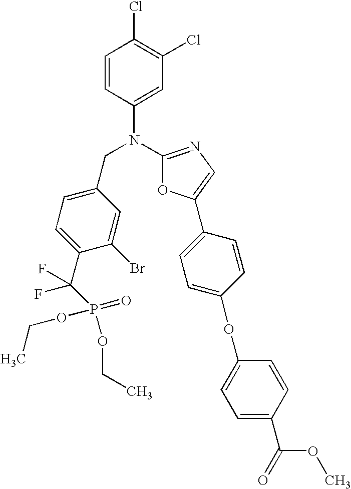 | 7 | 4-(4-{2-[{3-Bromo-4- [(diethoxy-phosphoryl)-difluoro- methyl]-benzyl}-(3,4-dichloro- phenyl)-amino]-oxazol-5-yl}- phenoxy)-benzoic acid methyl ester | + |
| 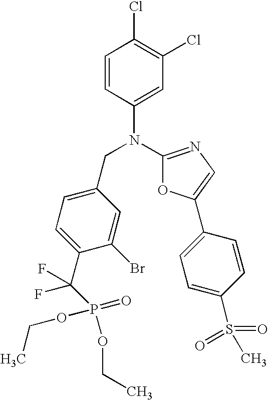 | 8 | {[2-Bromo-4-({(3,4-dichloro- phenyl)-[5-(4-methanesulfonyl- phenyl)-oxazol-2-yl]-amino}- methyl)-phenyl]-difluoro- methyl}-phosphonic acid diethyl ester | + |
| 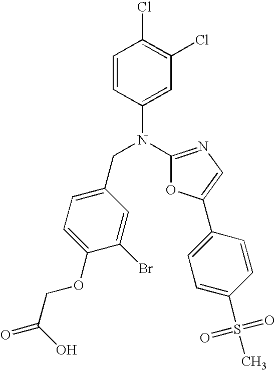 | | [2-Bromo-4-({(3,4-dichloro- phenyl)-[5-(4- methanesulfonyl-phenyl)- oxazol-2-yl]-amino}-methyl)- phenoxy]-acetic acid | + |
| 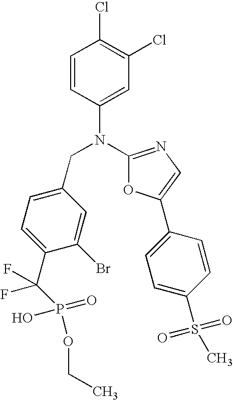 | 9 | {[2-Bromo-4-({(3,4-dichloro- phenyl)-[5-(4-methanesulfonyl- phenyl)-oxazol-2-yl]-amino}- methyl)-phenyl]-difluoro- methyl}-phosphonic acid monoethyl ester | + |
| 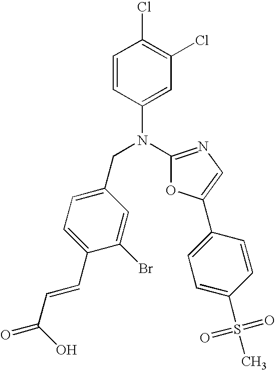 | | 3-[2-Bromo-4-({(3,4-dichloro- phenyl)-(5-(4- methanesulfonyl-phenyl)- oxazol-2-yl]-amino}-methyl)- phenyl]-acrylic acid | + |
| 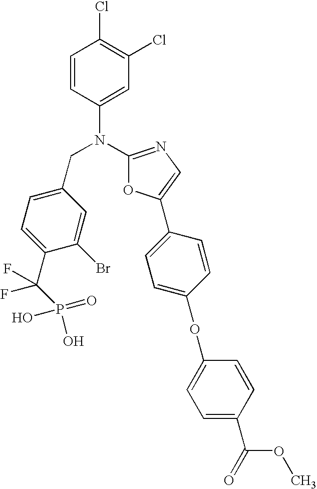 | 10 | 4-(4-{2-[[3-Bromo-4-(difluoro- phosphono-methyl)-benzyl]-(3,4- dichloro-phenyl)-amino]-oxazol- 5-yl}-phenoxy)-benzoic acid methyl ester | ++ |
| 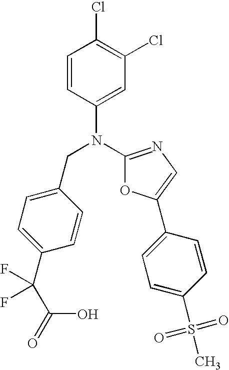 | | [4-({(3,4-Dichloro-phenyl)-[5- (4-methanesulfonyl-phenyl)- oxazol-2-yl]-amino}-methyl)- phenyl]-difluoro-acetic acid | + |
| 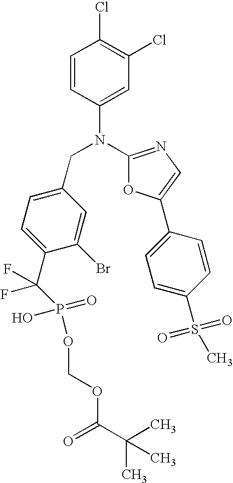 | 11 | 2,2-Dimethyl-propionic acid {[2- bromo-4-({(3,4-dichloro- phenyl)-[5-(4-methanesulfonyl- phenyl)-oxazol-2-yl]-amino}- methyl)-phenyl]-difluoro- methyl}-hydroxy- phosphinoyloxymethyl ester | + |
| 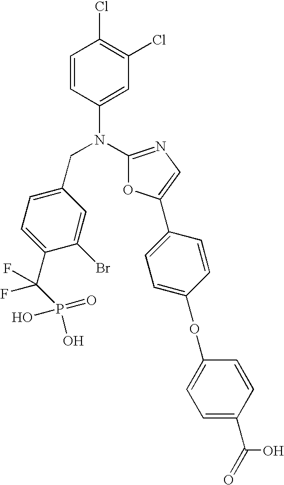 | 12 | 4-(4-{2-[[3-Bromo-4-(difluoro- phosphono-methyl)-benzyl]-(3,4- dichloro-phenyl)-amino]-oxazol- 5-yl}-phenoxy)-benzoic acid | ++++ |
| 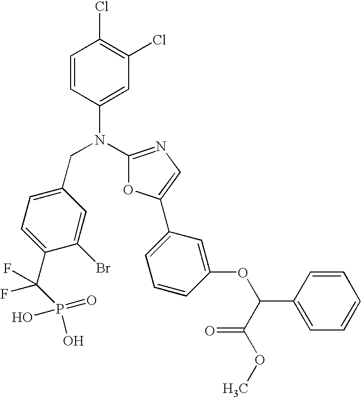 | 13 | (3-{2-[[3-Bromo-4-(difluoro- phosphono-methyl)-benzyl]-(3,4- dichloro-phenyl)-amino]-oxazol- 5-yl}-phenoxy)-phenyl-acetic acid methyl ester | ++ |
| 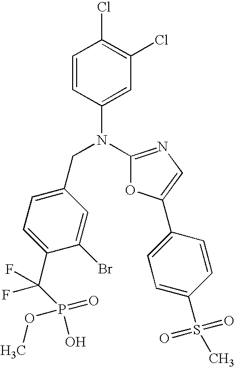 | | {[2-Bromo-4-({(3,4-dichloro- phenyl)-[5-(4- methanesulfonyl-phenyl)- oxazol-2-yl]-amino}-methyl)- phenyl]-difluoro-methyl]- phosphonic acid monomethyl ester | + |
| 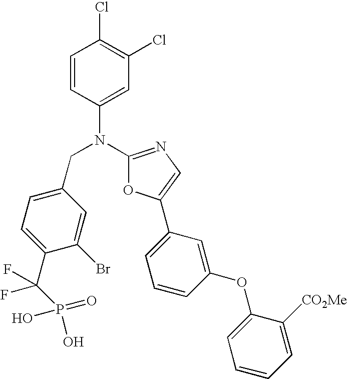 | 14 | 2-(3-{2-[[3-Bromo-4-(difluoro- phosphono-methyl)-benzyl]- (3,4-dichloro-phenyl)-amino]- oxazol-5-yl}-phenoxy)-benzoic acid methyl ester | ++ |
| 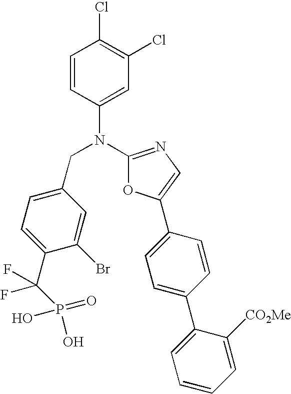 | 15 | 4′-{2-[[3-Bromo-4-(difluoro- phosphono-methyl)-benzyl]- (3,4-dichloro-phenyl)-amino]- oxazol-5-yl}-biphenyl-2- carboxylic acid methyl ester | ++ |
| 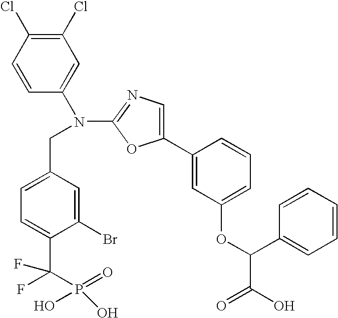 | 16 | (3-{2-[[3-Bromo-4-(difluoro- phosphono-methyl)-benzyl]- (3,4-dichloro-phenyl)-amino]- oxazol-5-yl}-phenoxy)-phenyl- acetic acid | +++++ |
| 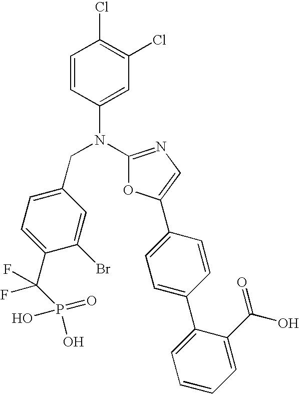 | 17 | 4′-{2-[[3-Bromo-4-(difluoro- phosphono-methyl)-benzyl]- (3,4-dichloro-phenyl)-amino]- oxazol-5-yl}-biphenyl-2- carboxylic acid | ++++ |
| 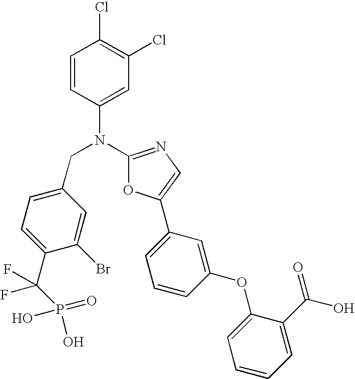 | 18 | 2-(3-{2-[[3-Bromo-4-(difluoro- phosphono-methyl)-benzyl]- (3,4-dichloro-phenyl)-amino]- oxazol-5-yl}-phenoxy)-benzoic acid | ++++ |
| 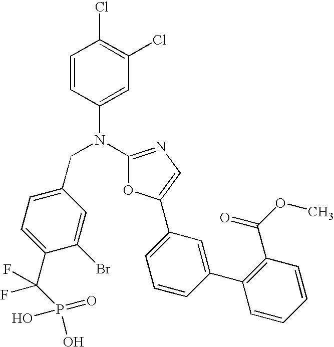 | 19 | 3′-{2-[[3-Bromo-4-(difluoro- phosphono-methyl)-benzyl]- (3,4-dichloro-phenyl)-amino]- oxazol-5-yl}-biphenyl-2- carboxylic acid methyl ester | ++ |
| 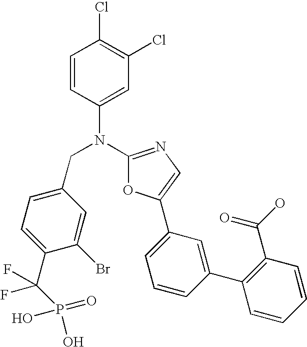 | 20 | 3′-{2-[[3-Bromo-4-(difluoro- phosphono-methyl)-benzyl]- (3,4-dichloro-phenyl)-amino]- oxazol-5-yl}-biphenyl-2- carboxylic acid | ++++ |
| 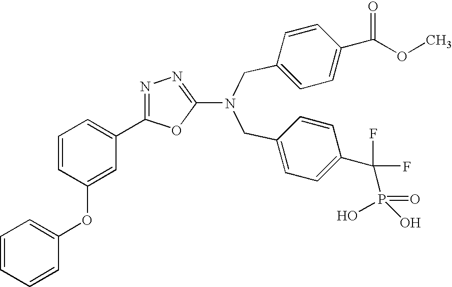 | | 4-({[4-(Difluoro-phosphono- methyl)-benzyl]-[5-(3- phenoxy-phenyl)- [1,3,4]oxadiazol-2-yl]-amino}- methyl)-benzoic acid methyl ester | + |
| 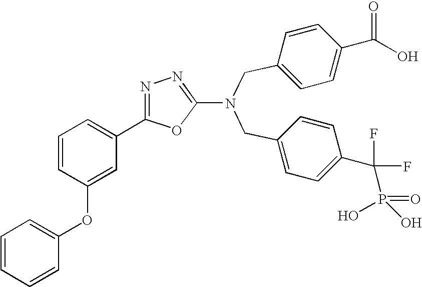 | | 4-({[4-(Difluoro-phosphono- methyl)-benzyl]-[5-(3- phenoxy-phenyl)- [1,3,4]oxadiazol-2-yl]-amino}- methyl)-benzoic acid | ++ |
| 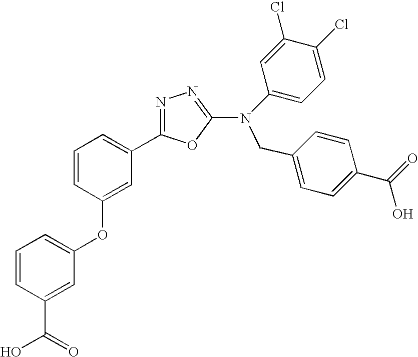 | 21 | 3-(3-{5-[[3-benzyl]-(3,4- dichloro-phenyl)-amino]-[1,3,4]oxadiazol-2-yl}-phenoxy)- benzoic acid | + |
| 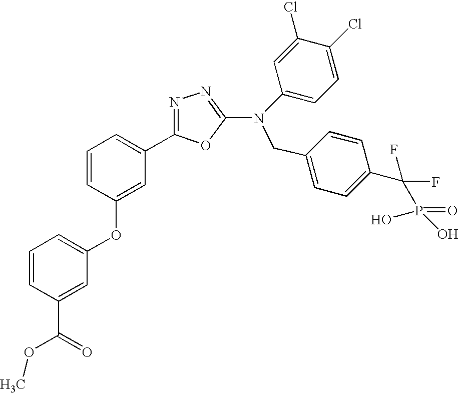 | 22 | 3-[3-(5-{(3,4-Dichloro-phenyl)- [4-(difluoro-phosphono-methyl)- benzyl]-amino}- [1,3,4]oxadiazol-2-yl)-phenoxy]- benzoic acid methyl ester | ++ |
| 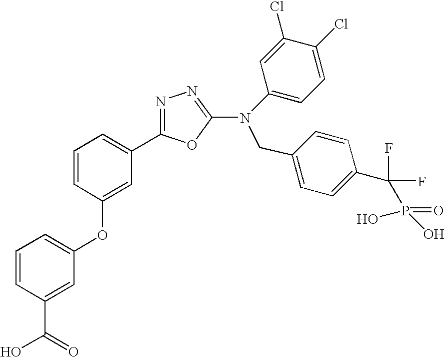 | 23 | 3-[3-(5-{(3,4-Dichloro-phenyl)- [4-(difluoro-phosphono-methyl)- benzyl]-amino}- [1,3,4]oxadiazol-2-yl)-phenoxy]- benzoic acid | ++ |
| 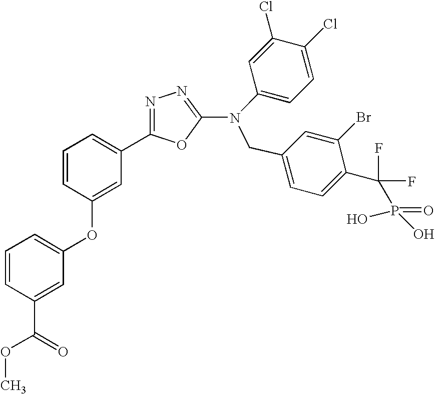 | 24 | 3-(3-{5-[[3-Bromo-4-(difluoro- phosphono-methyl)-benzyl]-(3,4- dichloro-phenyl)-amino]- [1,3,4]oxadiazol-2-yl}-phenoxy)- benzoic acid methyl ester | +++ |
| 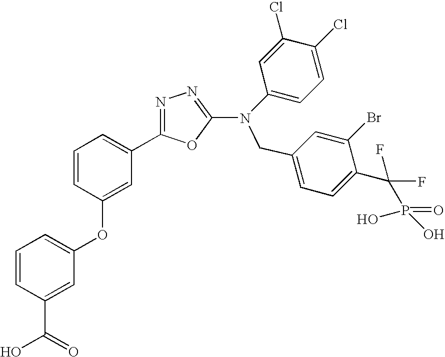 | 25 | 3-(3-{5-[[3-Bromo-4-(difluoro- phosphono-methyl)-benzyl]-(3,4- dichloro-phenyl)-amino]- [1,3,4]oxadiazol-2-yl}-phenoxy)- benzoic acid | ++++ |

TABLE 2
Table 2 depicts additional selected compounds provided herein.

| Structure | Example | Chemical Name | IC₅₀ |
|---|---|---|---|
| 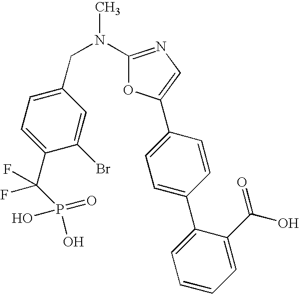 | 26 | 4′-{2-[[3-Bromo-4- (difluoro-phosphono- methyl)-benzyl]-methyl- amino]-oxazol-5-yl}- biphenyl-2-carboxylic acid | +++ |
| 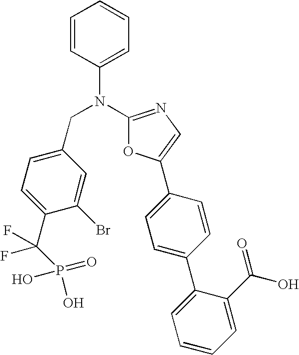 | 27 | 4′-{2-[[3-Bromo-4- (difluoro-phosphono- methyl)-benzyl]-phenyl- amino]-oxazol-5-yl}- biphenyl-2-carboxylic acid | ++++ |
| 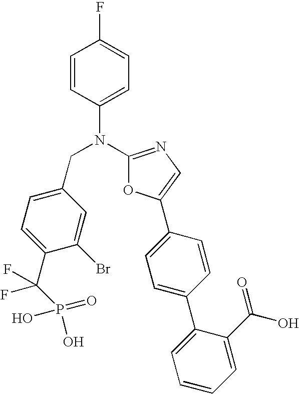 | 28 | 4′-{2-[[3-Bromo-4- (difluoro-phosphono- methyl)-benzyl]-(4- fluorophenyl)-amino]- oxazol-5-yl}-biphenyl-2- carboxylic acid | ++++ |
| 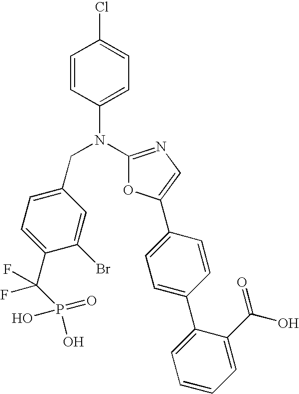 | 29 | 4′-{2-[[3-Bromo-4- (difluoro-phosphono- methyl)-benzyl]-(4- chlorophenyl)-amino]- oxazol-5-yl}-biphenyl-2- carboxylic acid | ++++ |
| 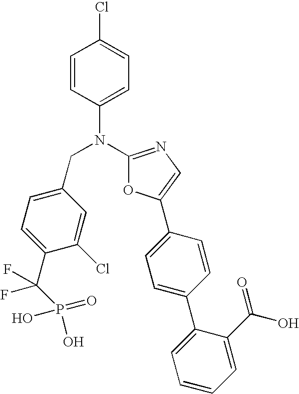 | 30 | 4′-{2-[[3-Chloro-4- (difluoro-phosphono- methyl)-benzyl]-(4- chlorophenyl)-amino]- oxazol-5-yl}-biphenyl-2- carboxylic acid | ++++ |
| 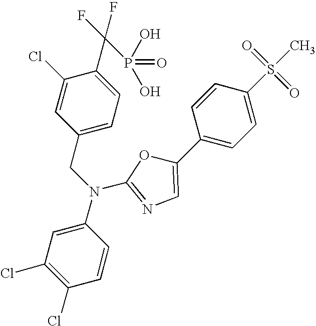 | 31 | {[2-Chloro-4-({(3,4- dichloro-phenyl)-[5-(4- methanesulfonyl- phenyl)-oxazol-2-yl]- amino}-methyl)-phenyl]- difluoro-methyl]- phosphonic acid | ++++ |
| 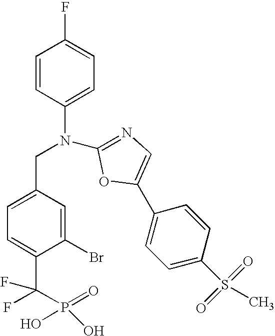 | 32 | {[2-Bromo-4-({(4-fluoro- phenyl)-[5-(4- methanesulfonyl- phenyl)-oxazol-2-yl]- amino}-methyl)-phenyl]- difluoro-methyl}- phosphonic acid | +++++ |
| 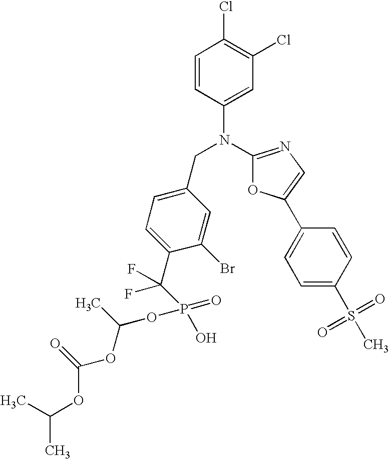 | 33 | {[2-Bromo-4-({(3,4- dichloro-phenyl)-[5-(4- methanesulfonyl- phenyl)-oxazol-2-yl]- amino}-methyl)-phenyl]- difluoro-methyl}- phosphonic acid mono- (1-isopropoxycarbonyloxy- ethyl) ester | + |
| 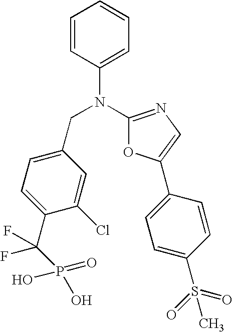 | — | {[2-Chloro-4-({(5-(4- methanesulfonyl- phenyl)-oxazol-2-yl]- phenyl-amino}- methyl)-phenyl]- difluoro-methyl}- phosphonic acid | ++++ |
| 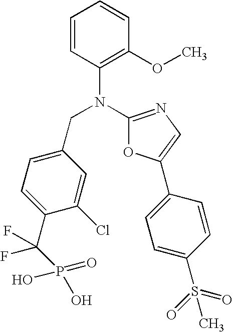 | — | [(2-Chloro-4-{[[5-(4- methanesulfonyl- phenyl)-oxazol-2-yl]-(2- methoxy-phenyl)- amino]-methyl}- phenyl)-difluoro- methyl]-phosphonic acid | +++ |
| 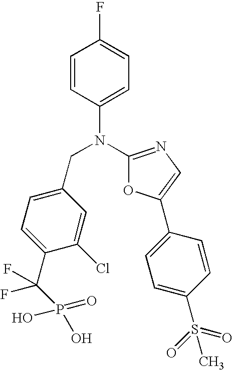 | — | {[2-Chloro-4-({(4-fluoro- phenyl)-[5- (4-methanesulfonyl- phenyl)-oxazol- 2-yl]-amino}-methyl)- phenyl]-difluoro- methyl}-phosphonic acid | ++++ |
| 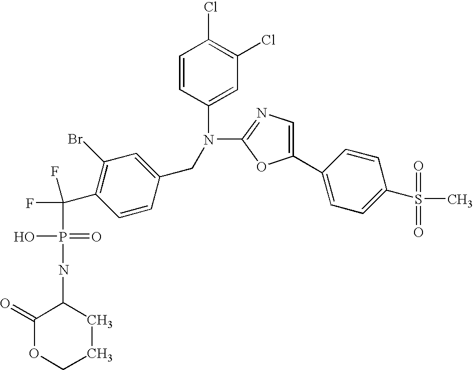 | 34 | {[2-Bromo-4-({(3,4- dichloro-phenyl)- [5-(4-methanesulfonyl- phenyl)-oxazol- 2-yl]-amino}-methyl)- phenyl]-difluoro- methyl}-phosphonic acid mono-(L-alanyl) amidate | ++++ |
| 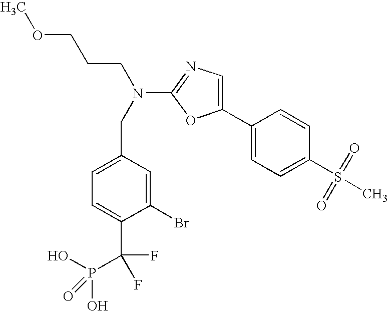 | — | [(2-Bromo-4-{[[5-(4- methanesulfonyl- phenyl)-oxazol-2-yl]-(3- methoxy-propyl)-amino]- methyl}-phenyl)- difluoro-methyl]-phosphonic acid | ++++ |
| 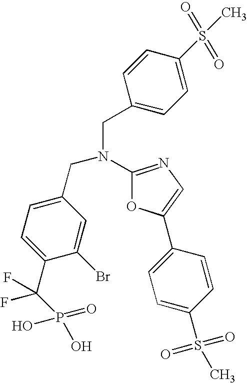 | — | {[2-Bromo-4-({(4- methanesulfonyl-benzyl)- [5-(4-methanesulfonyl-phenyl)- oxazol-2-yl]-amino}- methyl)-phenyl]-difluoro- methyl}-phosphonic acid | +++ |
| 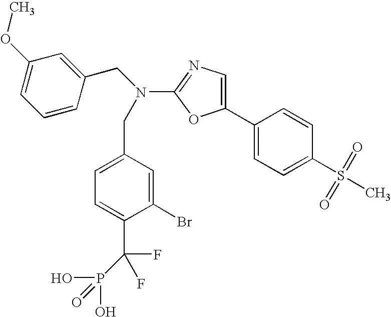 | — | [(2-Bromo-4-{[[5-(4- methanesulfonyl- phenyl)-oxazol-2-yl]-(3- methoxy-benzyl)-amino]- methyl}-phenyl)-difluoro- methyl]-phosphonic acid | ++++ |
| 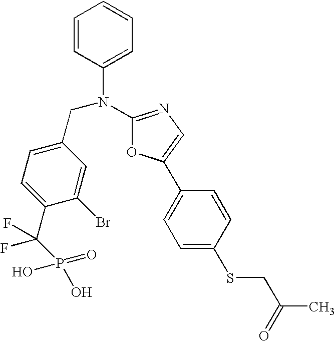 | — | {[2-Bromo-4-({[5-(4- carbamoylmethyl sulfanyl-phenyl)-oxazol- 2-yl]-phenyl-amino}- methyl)-phenyl]-difluoro- methyl}-phosphonic acid | +++++ |
| 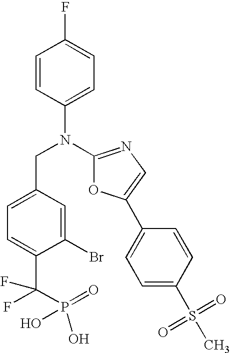 | 36 | {[2-Bromo-4-({(4-fluoro- phenyl)-[5-(4- methanesulfonyl- phenyl)-oxazol-2-yl]- amino}-methyl)-phenyl]- difluoro-methyl}- phosphonic acid | +++++ |
| 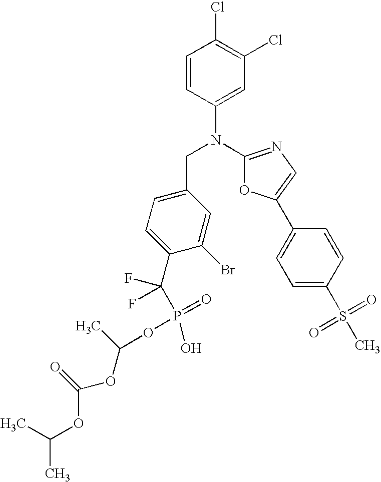 | — | {[2-Bromo-4-({(3,4- dichloro-phenyl)-[5-(4- methanesulfonyl- phenyl)-oxazol-2-yl]- amino]-methyl)-phenyl]- difluoro-methyl}- phosphonic acid mono-(1- isopropoxycarbonyloxy- ethyl) ester | + |
| 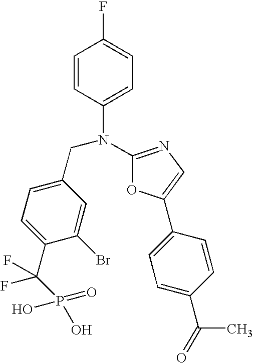 | 37 | [(2-Bromo-4-{[[5-(4- carbamoyl-phenyl)- oxazol-2-yl]-(4-fluoro- phenyl)-amino]-methyl}- phenyl)-difluoro-methyl]- phosphonic acid | ++++ |
| 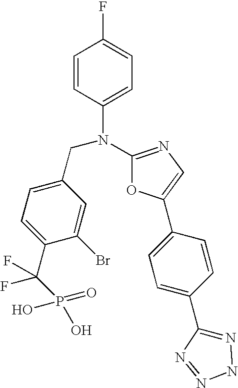 | 38 | ({2-Bromo-4-[((4-fluoro- phenyl)-{5-[4-(2H- tetrazol-5-yl)-phenyl]- oxazol-2-yl}-amimo)- methyl]-phenyl}-difluoro- methyl)-phosphonic acid | +++++ |
| 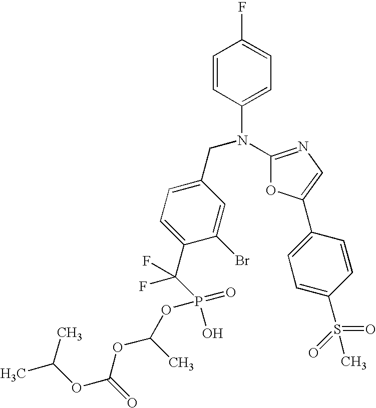 | — | {[2-Bromo-4-({(4-fluoro- phenyl)-[5-(4- methanesulfonyl- phenyl)-oxazol-2-yl]- amino}-methyl)-phenyl]- difluoro-methyl}- phosphonic acid mono-(1- isopropoxycarbonyloxy- ethyl) ester | + |
| 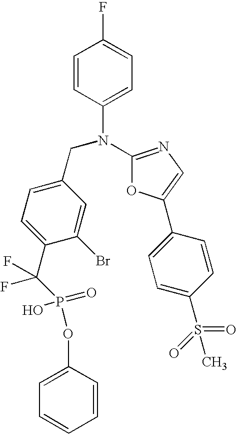 | — | {[2-Bromo-4-({(4-fluoro- phenyl)-[5-(4- methanesulfonyl- phenyl)-oxazol-2-yl]- amino}-methyl)-phenyl]- difluoro-methyl}- phosphonic acid monophenyl ester | + |
| 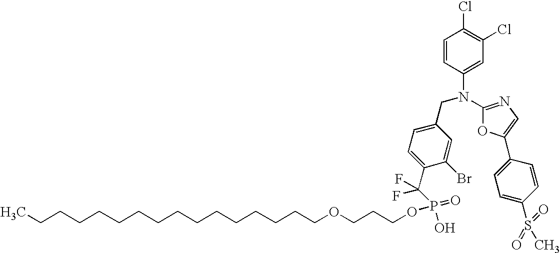 | — | {[2-Bromo-4-({(3,4- dichloro-phenyl)-[5-(4- methanesulfonyl- phenyl)-oxazol-2-yl]- amino}-methyl)-phenyl]- difluoro-methyl}- phosphonic acid mono- (3-hexadecyloxy-propyl) ester | ++ |
| 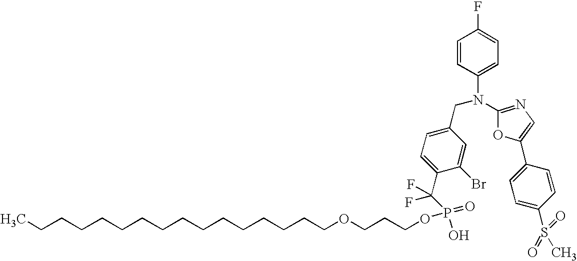 | — | {[2-Bromo-4-({(4-fluoro- phenyl)-[5-(4- methanesulfonyl- phenyl)-oxazol-2-yl]- amino}-methyl)-phenyl]- difluoro-methyl}- phosphonic acid mono- (3-hexadecyloxy-propyl) ester | ++ |
| 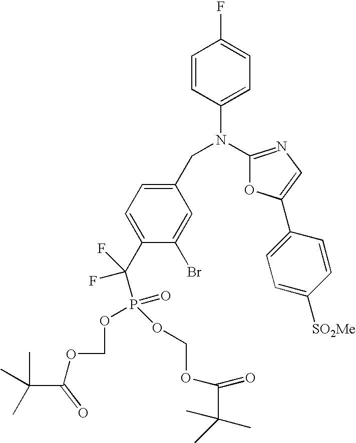 | 35 | 2,2-Dimethyl-propionic acid {[2-bromo-4-({(4- fluoro-phenyl)-[5-(4- methanesulfonylphenyl)- oxazol-2-yl]-amino}- methyl)-phenyl]-difluoro- methyl}-(2,2-dimethyl- propionyloxymethoxy)- phosphinoyloxymethyl ester | + |

+ 10-100 micromolar
++ 1-10 micromolar
+++ 0.5-1 micromolar
++++ <0.1-0.5 micromolar
+++++ <0.1 micromolar

Mass spectral data for certain of the compounds provided herein is shown in the Table below.

| Structure | m/z for [M + H]+ | m/z for [M − H]− |
|---|---|---|
| 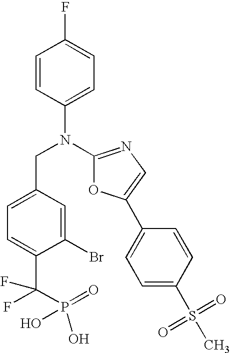 | | 630.7 |
| 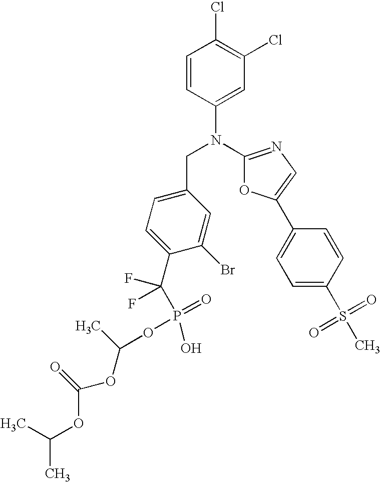 | | 811.7 |
| 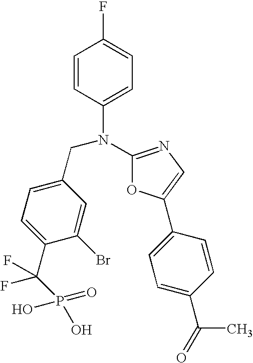 | | 595.8 |
| 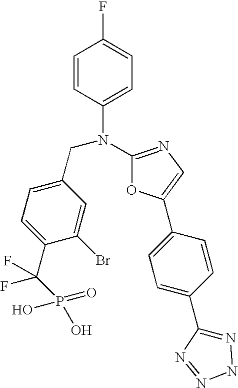 | | 620.8 |
| 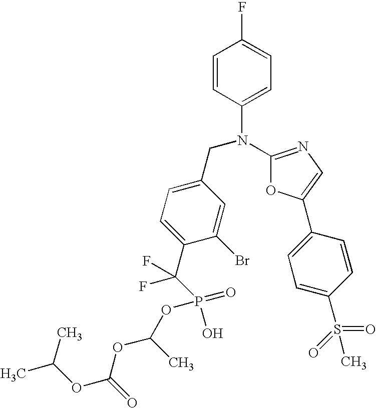 | 763 | 761 |
| 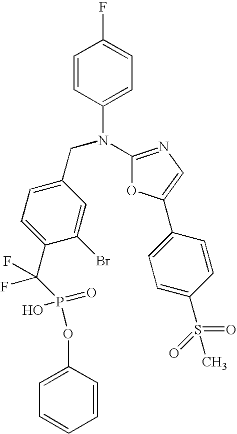 | 707 | 705 |
| 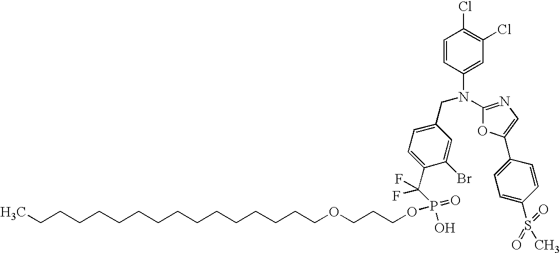 | | 962.8 |
| 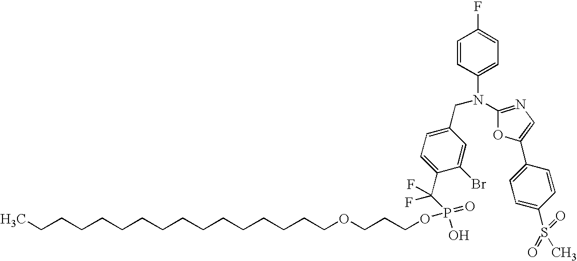 | | 912.8 |

### EXAMPLES

In the experimental disclosure which follows, all weights are given in grams (g), milligrams (mg), micrograms (μg), nanograms (ng), or picograms (pg), all amounts are given in moles (mol), millimoles (mmol), micromoles (μmol), nanomoles (nmol), picomoles (pmol), or femtomoles (fmol), all concentrations are given as percent by volume (%), proportion by volume (v:v), molar (M), millimolar (mM), micromolar (μM), nanomolar (nM), picomolar (pM), femtomolar (fM), or normal (N), all volumes are given in liters (L), milliliters (mL), or microliters (μL), and linear measurements are given in millimeters (mm), micrometers (pm), or nanometers (nm) and mp is melting point, unless otherwise indicated.

Procedure A

4-(2-Bromo-acetyl)-methyl benzoate: To a solution of methyl-4-acetyl benzoate (5 g, 28 mmol) in 50 mL of CHCl₃ was added bromine (4.48 g, 28 mmol) in 15 mL of CHCl₃. When the reaction was complete, water was added carefully to the reaction mixture and the organic layer was washed with sat. NaHCO₃ and brine. The organic layer was dried over MgSO₄, and concentrated in vacuo to yield 4-(2-Bromo-acetyl)-methyl benzoate, which was used in the next step without purification.

¹H NMR (300 MHz, CDCl₃)δ 8.40 (s, 1H), 8.13 (d, J=8.0 Hz, 2H), 8.02 (d, J=7.8 Hz, 2H), 4.46 (s, 2H); 3.95 (s, 3H).

Procedure B

2-Azido-1-phenyl-ethanone: To a solution of 2-bromo-1-phenyl-ethanone (1.0 g, 5.02 mmol) in acetone (6 mL) and water (3 mL) was added sodium azide and the reaction mixture was heated at 50° C. for 20 min. The reaction mixture was concentrated under reduced pressure, azeotroped with toluene (2×10 mL) and taken on to the next step without any further purification.

Procedure C

(3,4-Dichloro-phenyl)-(5-phenyl-oxazol-2-yl)-amine: To a solution of 2-azido-1-phenyl-ethanone in anhydrous dioxane (10 mL) was added the 1,2-dichloro-4-isothiocyanato-benzene (0.85 g, 4.18 mmol) and triphenylphosphine (1.31 g, 5.02 mmol) and the reaction mixture was heated at 90° C.-100° C. for 25 min. The reaction mixture was allowed to cool to room temperature and concentrated under vacuum and partitioned between ethyl acetate and water. The solid that precipitated out was washed with water and cold ethyl acetate to yield 0.9 g (71%) of an off-white solid.

¹H NMR (300 MHz, DMSO-d₆)δ 8.08 (s, 1H), 7.67-7.45(m, 10H), 7.30 (s, 1H); LCMS m/z 304 [M⁻]

Procedure D

[(2-Bromo-4-{[(3,4-dichloro-phenyl)-(5-phenyl-oxazol-2-yl)-amino]-methyl}-phenyl)-difluoro-methyl]-phosphonic acid diethyl ester: To (3,4 -Dichloro-phenyl)-(5-phenyl-oxazol-2-yl)-amine (0.20 g, 0.655 mmol) in DMF (5 mL) was added K₂CO₃ (0.45 g, 3.2 mmol). After 0.1 hour, [(2-Bromo-4-bromomethyl-phenyl)-difluoro-methyl]-phosphonic acid diethyl ester (0.287 g, 0.655 mmol) was added and the resulting mixture was stirred at room temperature overnight. The reaction was partitioned between ethyl acetate and H₂O, after which the organic layer was dried over NaSO₄ and concentrated in vacuo. The resulting material was purified via column chromatography (1/1 hexanes/ethyl acetate) to yield 0.270 g (63%) of a clear, colorless oil.

¹H NMR (300 MHz, CDCl₃) δ 7.65-7.13 (m, 12 H), 5.16 (s, 2 H), 4.21 (m, 4 H), 1.31 (t, J=7.2 Hz, 6 H).

Procedure E

[(2-Bromo-4-{[(3,4-dichloro-phenyl)-(5-phenyl-oxazol-2-yl)-amino]-methyl}-phenyl)-difluoro-methyl]-phosphonic acid: To [(2-Bromo-4-{[(3,4-dichloro-phenyl)-(5-phenyl-oxazol-2-yl)-amino]-methyl}-phenyl)-difluoro-methyl]-phosphonic acid diethyl ester (0.110 g, 0.166 mmol) in CH₂Cl₂ (3 mL) was added bistrimethylsilyltrifluoroacetamide (0.429 g, 1.66 mmol) and reaction mixture was stirred at room temperature for 1 hour after which the reaction mixture is cooled to 20° C. and iodotrimethylsilane (0.332 g, 1.66 mmol) is added drop wise. The resulting mixture was stirred at room temperature for 1.5 hours, after which it was concentrated in vacuo. The resulting material was stirred in CH₃ CN (4 mL), H₂O (0.5 mL), and TFA (0.5 mL) for 0.5 hours, after which it was concentrated in vacuo and partitioned between ethyl acetate and acidic Na₂S₂O₄. The organic layer was dried over MgSO₄ and concentrated in vacuo to yield 0.090 g (90%) of white foam.

¹H NMR (300 MHz, DMSO-d₆ ) δ 7.99-7.28 (m, 12 H), 5.33 (s, 2 H); LCMS m/z 604 [M⁺¹]

Procedure F

(4-{2-[[3-Bromo-4-(difluoro-phosphono-methyl)-benzyl]-(3,4-dichloro-phenyl)-amino]-thiazol-4-yl}-benzoic acid: To 4-{2-[[3-Bromo-4-(difluoro-phosphono-methyl)-benzyl]-(3,4-dichloro-phenyl)-amino]-thiazol-4-yl}-methyl benzoate (0.99 g, 0.146 mmol) in 3 mL each of THF and methanol was added a 2.5N solution of NaOH (5 equiv) and the reaction was stirred at room temperature overnight and concentrated in vacuo. The residue was washed with ethyl acetate, and then 15% HCl aqueous solution was added until the pH was 2. The water layer was extracted with ethyl acetate and washed with brine. Concentration in vacuo gave the title compound, (4-{2-[[3-Bromo-4-(difluoro-phosphono-methyl)-benzyl]-(3,4-dichloro-phenyl)-amino]-thiazol-4-yl}-benzoic acid in 90% yield.

Procedure G

A mixture of 4-bromobenzaldehyde (1.000 g; 5.4 mmol), methyl 4-hydroxybenzoate (987 mg; 6.5 mmol) and potassium carbonate (1.494 g; 10.8 mmol) in dry pyridine (8 mL) is stirred under argon at RT. Copper (II) oxide (860 mg; 10.8 mmol) is added and the reaction mixture is refluxed for 12 hours. After cooling to RT, CH₂Cl₂ (50 mL) is added and the mixture is filtered through celite. The filter cake is washed with fresh CH₂Cl₂ (50 mL). The combined organics are concentrated in vacuo. The residue is purified by flash chromatography (ethyl acetate/hexanes, 1:10 to 1:4 ) to yield methyl 3-(3-carbonylphenoxy)benzoate (776 mg; 56%) as a yellow oil.

¹H NMR (300 MHz, CDCl₃): δ 9.97 (1H, s), 7.85 (1H, d, J=6.9 Hz), 7.69-7.24 (7H, m), 3.91 (3H, s).

Procedure H

[(2-Bromo-4-methyl-phenyl)-difluoromethyl]-phosphonic acid diethyl ester: To activated zinc (1.2 g, 19 mmol) in DMA (7 mL) was added bromodifluoromethyldiethyl-phosphonate (5.0 g, 19 mmol) in DMA (7 mL). The resulting mixture was stirred at 45° C. for 3 hours, after which copper (I) bromide (2.7 g, 19 mmol) was added and stirring was continued for 0.5 hours at room temperature. 3-Bromo-4-iodotoluene (2.8 g, 9.4 mmol) was then added and the mixture was sonicated at room temperature for 12 hours. The reaction mixture was partitioned between ether and H₂O, filtered through Celite, and the organic layer was dried over MgSO₄ and concentrated in vacuo to yield 2.1 g (63%) of a clear, colorless oil.

¹H NMR (300 MHz, CDCl₃) δ 7.51 (d, J=6 Hz, 1 H), 7.27 (s, 1 H), 7.20 (d, J=7.5 Hz, 1 H), 4.27 (m, 4 H), 1.36 (t, J=8.1 Hz).

Procedure I

[(2-Bromo-4-bromomethyl-phenyl)-difluoro-methyl]-phosphonic acid diethyl ester. To [(2-Bromo-4-methyl-phenyl)-difluoromethyl]-phosphonic acid diethyl ester (2.1 g, 5.8 mmol) in benzene (50 mL) was added N-bromosuccinimide (1.2 g, 6.8 mmol) and AIBN (0.050 g). The resulting mixture was stirred for 12 hours at room temperature in front of a 100 W bulb. It was then washed with H₂O, sat. NaHCO₃ , and brine, and the organic layer was dried over MgSO4, and concentrated in vacuo. The resulting material was purified via column chromatography (4/1 hexanes/ethyl acetate) to yield 1.7 g (66%) of clear, colorless oil.

¹H NMR (300 MHz, CDCl₃) δ 7.71 (s, 1 H), 7.61 (d, J=8.1 Hz, 2 H), 7.41 (d, J=8.1 Hz, 1 H), 4.41 (s, 2 H), 4.27 (m, 4 H), 1.36 (t, J=8.1 Hz).

Procedure J

3-(3-{5-[[3-benzyl]-(3,4-dichloro-phenyl)-amino]-[1,3,4 ]oxadiazol-2-yl}-phenoxy)-benzoic acid: To 3-{3-[5-(3,4-Dichloro-phenylamino)-[1,3,4 ]oxadiazol-2-yl]-phenoxy}-benzoic acid methyl ester (0.297 g, 0.651 mmol) in DMF (2.5 ml) was added K₂CO₃ (0.454 g, 3.28 mmol.) followed by 4-Bromomethyl-benzoic acid methyl ester (0.151 g, 0.660 mmol.). The reaction mixture was stirred for 14 hours at room temperature, after which it was partitioned between water and ethyl acetate. The aqueous layer was extracted with 3×10 ml ethyl acetate and the combined organic layers were washed with brine, dried over Na₂ SO₄ and concentrated in vacuo to yield a yellow oil which was purified by column chromatography (ethyl acetate:hexanes, 1:8 to 1:2). Two isomers were obtained as white solids. Normal isomer (0.354 g, 90%) (1:2 ethyl acetate:hexanes, R_{f}=0.08).

¹H NMR (300 MHz, DMSO-d₆): δ 7.97-7.96 (m, 1H), 7.92-7.89 (m, 2H), 7.80-7.78 (m, 1H), 7.67-7.49 (m, 8H), 7.41-7.40 (m, 2H), 7.27-7.25 (m, 1H), 5.38 (s, 2H), 3.84 (s, 3H), 3.83 (s, 3H). Iso compound (19.0 mg, 5.0%) (1:2 ethyl acetate:hexanes, R_{f}=0.25); ¹H NMR (300 MHz, DMSO-d₆): δ 8.05-8.03 (m, 2H), 7.85-7.83 (m, 1H), 7.66 (s, 1H), 7.57-7.42 (m, 6H), 7.34-7.31 (m, 2H), 7.26-7.22 (m, 1H), 7.13-7.05 (m, 2H), 5.09 (s, 2H), 3.90 (s, 6H).

Procedure K

2-(3-{2-[[3-Bromo-4-(difluoro-phosphono-methyl)-benzyl]-(3,4-dichloro-phenyl)amino]-oxazol-5-yl}-phenoxy)-benzoic acid methyl ester: 3-hydroxyacetophenone (5.00 g, 36.72 mmol) and methyl-2-bromo-benzoate (6.58 g, 30.60 mmol) were dissolved in 15 mL dry pyridine. Copper (II) oxide (4.87 g, 61.21 mmol) and potassium carbonate (8.46 g, 61.21 mmol) were added and the black suspension refluxed under nitrogen for 16 hours. The mixture was diluted with dichloromethane, filtered through celite, and concentrated in vacuo. Purification by silica gel flash chromatography yielded 2-(3-Acetyl-phenoxy)-benzoic acid methyl ester as an orange oil (4.45 g, 54%).

¹H NMR (CDCl₃, 600 MHz): δ 2.57 (s, 3H), 3.80 (s, 3H), 7.01 (d, J=8.4 Hz, 1H), 7.16 (dd, J=7.8 Hz, 1.8 Hz, 1H), 7.24 (t, J=6.6 Hz, 1H), 7.42 (t, J=7.8 Hz, 1H), 7.51 (m, 2H), 7.67 (d, J=7.8 Hz, 1H), 7.96 (dd, J=7.8 Hz, 1.8 Hz, 1H); LCMS m/z 271 (M+1).

Procedure L

4′-Acetyl-biphenyl-2-carboxylic acid methyl ester: (4-Acetyl)-phenylboronic acid (7.62 g, 46.50 mmol), 2-bromobenzoic acid methyl ester (10.0 g, 46.50 mmol), dichlorobis-(triphenylphosphine)palladium (450 mg, 0.64 mmol), and sodium carbonate (solution in 20 mL water) were combined in 50 mL 4:1 dimethoxyethane/ethanol. The heterogeneous mixture was heated in a microwave at 140° C. for 25 min. The mixture was diluted with water then extracted twice with ethyl acetate. The combined organic layers were dried (MgSO₄) then concentrated in vacuo. Purification by silica gel flash chromatography yielded the title compound as yellow oil, 9.3 g (79%).

¹H NMR (CDCl₃, 600 MHz): δ 2.64 (s, 3H), 3.66 (s, 3H), 7.36 (d, J=7.8 Hz, 1H), 7.40 (d, J=7.2 Hz, 2H), 7.46 (t, J=7.8 Hz, 1H), 7.57 (t, J=7.8 Hz, 1H), 7.90 (d, J=7.8 Hz, 1H), 8.00 (d, J=7.8 Hz, 2H); MS (ESI): m/z 255 (M+1).

### EXAMPLE 1

### [(2-Bromo-4-{[(3,4-dichloro-phenyl)-(5-phenyl-oxazol-2-yl)-amino]-methyl}-phenyl)-difluoro-methyl]-phosphonic acid

As described in procedure B, 2-Azido-1-phenyl-ethanone was prepared. As described in procedure C, (3,4-Dichloro-phenyl)-(5-phenyl-oxazol-2-yl)-amine was prepared. [(2-Bromo-4-{[(3,4-dichloro-phenyl)-(5-phenyl-oxazol-2-yl)-amino]-methyl}-phenyl)-difluoro-methyl]-phosphonic acid diethyl ester was prepared as described in procedure D. The title compound was prepared as described in procedure E. ¹H NMR (300 MHz, DMSO-d₆) δ 7.99-7.28 (m, 12 H), 5.33 (s, 2H); LCMS m/z 604 [M⁺¹]

### EXAMPLE 2

### 4-{2-[[3-Bromo-4-(difluoro-phosphono-methyl)-benzyl]-(3,4-dichloro-phenyl)-amino]-oxazol-5-yl}-benzoic acid methyl ester

The title compound, 4-{2-[[3-Bromo-4-(difluoro-phosphono-methyl)-benzyl]-(3,4-dichloro-phenyl)-amino]-oxazol-5-yl}-benzoic acid methyl ester was synthesized as described for the compound of Example 1 and using procedure A (for the synthesis of 4-(2-Bromo-acetyl)-methyl benzoate). ¹H NMR (300 MHz, DMSO-d₆) δ 7.97-7.40 (m, 11 H), 5.33 (s, 2H), 3.88 (s, 3H); LCMS m/z 328.2 [M/2-1].

### EXAMPLE 3

### 4-{2-[[3-Bromo-4-(difluoro-phosphono-methyl)-benzyl]-(3,4-dichloro-phenyl)-amino]-oxazol-5-yl}-benzoic acid

The title compound 4-{2-[[3-Bromo-4-(difluoro-phosphono-methyl)-benzyl]-(3,4-dichloro-phenyl)-amino]-oxazol-5-yl}-benzoic acid, was synthesized as described in procedure F. ¹H NMR (300 MHz, DMSO-d₆) δ 7.97-7.40 (m, 11 H), 5.33 (s, 2H); LCMS m/z 322 [M/2-1].

### EXAMPLE 4

### 4-(2-{[3-Bromo-4-(difluoro-phosphono-methyl)-benzyl]-phenyl-amino}-oxazol-5-yl)-benzoic acid methyl ester

The title compound, 4-(2-{[3-Bromo-4-(difluoro-phosphono-methyl)-benzyl]-phenyl-amino}-oxazol-5-yl)-benzoic acid methyl ester was synthesized as described for the compound of Example 2.

¹H NMR (300 MHz, DMSO-d₆) δ 7.95-7.18 (m, 13 H), 5.28 (s, 2H), 3.83 (s, 3H); LCMS m/z 594 [M+1]

### EXAMPLE 5

### 4-(2-{[3-Bromo-4-(difluoro-phosphono-methyl)-benzyl]-phenyl-amino}-oxazol-5-yl)-benzoic acid

The title compound 4-(2-{[3-Bromo-4-(difluoro-phosphono-methyl)-benzyl]-phenyl-amino}-oxazol-5-yl)-benzoic acid, was synthesized as described for the compound of Example 3.

¹H NMR (300 MHz, DMSO-d₆) δ 7.94-7.19 (m, 13 H), 5.28 (s, 2H); LCMS m/z 580 [M+1].

### EXAMPLE 6

### {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid

The title compound {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid was synthesized as described for the compound of Example 1.

¹H NMR (300 MHz, DMSO-d₆) δ 7.98-7.39 (m, 11 H), 5.33 (s, 2H), 3.19 (s, 3H); LCMS m/z 683 [M+1]

### EXAMPLE 7

### 4-(4-{2-[{3-Bromo-4-[(diethoxy-phosphoryl)-difluoro-methyl]-benzyl}-(3,4-dichloro-phenyl)-amino]-oxazol-5-yl}-phenoxy)-benzoic acid methyl ester

The title compound was synthesized using procedure K and procedure A-C.

¹H NMR (300 MHz, CDCl₃): δ 8.01 (d, 2H, J=4.2 Hz), 7.64-6.99 (m, 14 H), 5.15 (s, 2H), 4.26-4.11 (m, 4 H), 3.90 (s, 3H), 1.33-1.24 (t, 6H, J=3.3 Hz); LCMS m/z 811 [M+1].

### EXAMPLE 8

### {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid diethyl ester

The title compound was synthesized using procedures B-E.

¹H NMR (300 MHz, CDCl₃): δ 7.91 (d, 2H, J=4.2 Hz), 7.64-7.20 (m, 9 H), 5.16 (s, 2H), 4.25-4.20 (m, 4 H), 3.05 (s, 3H), 1.33-1.31 (t, 6H, J=3.3 Hz); LCMS m/z 739 [M+1].

### EXAMPLE 9

### {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monoethyl ester

To (0.076 g, 0.102 mmol) of the compound of Example 8 in 3 mL each of THF and methanol was added a 2.5N solution of LiOH (5 equiv) and the reaction was stirred at room temperature overnight and concentrated in vacuo. The residue was washed with ethyl acetate, and then 15% HCl aqueous solution was added until the pH was 2. The water layer was extracted with ethyl acetate and washed with brine. Concentration in vacuo gave the title compound, {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monoethyl ester in 90% yield.

¹H NMR (300 MHz, DMSO- d₆): δ 7.97-7.41 (m, 11 H), 5.34 (s, 2H), 3.88 (m, 2H), 3.20 (s, 3H), 1.33-1.10 (m, 3H); LCMS m/z 710 [M+1].

### EXAMPLE 10

### 4-(4-{2-[[3-Bromo-4-(difluoro-phosphono-methyl)-benzyl]-(3,4-dichloro-phenyl)-amino]-oxazol-5-yl}-phenoxy)-benzoic acid methyl ester

The title compound 4-(4-{2-[[3-Bromo-4-(difluoro-phosphono-methyl)-benzyl]-(3,4-dichloro-phenyl)-amino]-oxazol-5-yl}-phenoxy)-benzoic acid methyl ester was synthesized from the compound of Example 7 using procedure E.

¹H NMR (300 MHz DMSO- d₆): δ 7.97-7.07 (m, 15 H), 5.33 (s, 2H), 3.83 (s, 3H); LCMS m/z 753 [M+1]

### EXAMPLE 11

### 2,2-Dimethyl-propionic acid {[2-bromo-4-({(3,4-dichloro-phenyl)-[5-(4-methanesulfonyl-phenyl)-oxazol-2-yl]-amino}-methyl)-phenyl]-difluoro-methyl}-hydroxy-phosphinoyloxymethyl ester

To (0.059 mg, 0.086 mmol) of the compound of Example 6 in 6 mL of DMF was added (0.029 mL, 0.172 mmol) of DIEA and (0.025 mL, 0.172 mmol) of pivaloyloxymethyl chloride and the solution is heated at 60° C. overnight. The reaction mixture was partitioned in EtOAc (150 mL) and organic layer was washed with H₂O, and brine. The organic phase was dried over Na₂SO₄ and concentrated in vacuo to yield a yellow solid which was purified by triturating in hexanes to afford the desired product (0.053 g, 78%) as an off-white solid.

¹H NMR (300 MHz, DMSO- d₆): δ 7.97-7.32 (m, 11 H), 5.36 (d, 2H, J=5.1 Hz), 5.32 (s, 2H), 3.20 (s, 3H), 1.07 (s, 9 H); LCMS m/z 795 [M−1].

### EXAMPLE 12

### 4-(4-{2-[[3-Bromo-4-(difluoro-phosphono-methyl)-benzyl]-(3,4-dichloro-phenyl)-amino]-oxazol-5-yl}-phenoxy)-benzoic acid

The title compound 4-(4-{2-[[3-Bromo-4-(difluoro-phosphono-methyl)-benzyl]-(3,4-dichloro-phenyl)-amino]-oxazol-5-yl}-phenoxy)-benzoic acid was synthesized from the compound of Example 10 using procedure F.

¹H NMR (300 MHz, D₂0): δ 7.68-6.74 (m, 15 H), 4.94 (s, 2H); LCMS m/z 740 [M+1].

### EXAMPLE 13

### (3-{2-[[3-Bromo-4-(difluoro-phosphono-methyl)-benzyl]-(3,4-dichloro-phenyl)-amino]-oxazol-5-yl}-phenoxy)-phenyl-acetic acid methyl ester

To 3-hydroxy acetophenone (5 g, 36.72 mmol) in 100 mL of acetone was added K₂CO₃ (6.09 g, 44.06 mmol) followed by the addition of methyl α-bromo phenyl acetate (10 g, 44.06 mmol) and the reaction was stirred at room temperature overnight. The reaction mixture was filtered, concentrated and purified via column chromatography (5/1 hexanes/ethyl acetate) to yield 8.15 g (84%) of (3-acetyl-phenoxy)-phenyl-acetic acid methyl ester as a clear, colorless oil.

¹H NMR (600 MHz, CDCl₃) δ δ 7.60-7.17 (m, 9 H), 5.74 (s, 1H), 3.76 (s, 3H), 2.59 (s,3 H).

[3-(2-Bromo-acetyl)-phenoxy]-phenyl-acetic acid methyl ester was synthesized from (3-acetyl-phenoxy)-phenyl-acetic acid methyl ester and bromine as described in procedure A.

{3-[2-(3,4-Dichloro-phenylamino)-oxazol-5-yl]-phenoxy}-phenyl-acetic acid methyl ester was prepared as per procedure B and C, using 1,2-dichloro-4-isothiocyanato-benzene and [3-(2-Bromo-acetyl)-phenoxy]-phenyl-acetic acid methyl ester. (3-{2-[[3-Bromo-4-(difluoro-phosphono-methyl)-benzyl]-(3,4-dichloro-phenyl)-amino]-oxazol-5-yl}-phenoxy)-phenyl-acetic acid methyl ester was synthesized as per procedure D and E.

¹H NMR (300 MHz, DMSO- d₆): δ 7.97-6.90 (m, 16 H), 6.10 (s, 1H), 5.35 (s, 2H), 3.65 (s, 3H); LCMS m/z 769 [M+1].

### EXAMPLE 14

### 2-(3-{2-[[3-Bromo-4-(difluoro-phosphono-methyl)-benzyl]-(3,4-dichloro-phenyl)amino]-oxazol-5-yl}-phenoxy)-benzoic acid methyl ester

2-(3-Acetyl-phenoxy)-benzoic acid methyl ester as an orange oil was prepared as described in procedure K.

2-{3-[2-(3,4-Dichloro-phenylamino)-oxazol-5-yl]-phenoxy}-benzoic acid methyl ester was prepared as described in procedures A-C.

¹H NMR (DMSO-d₆, 600 MHz): δ 3.73 (s, 3H), 6.85 (dd, J=7.2 Hz, 1.8 Hz, 1H), 7.13 (m, 2H), 7.33-7.65 (m, 7 H), 7.87 (d, J=7.2 Hz, 1H), 8.01 (d, J=1.8 Hz, 1H), 10.72 (s, 1H); LCMS: m/z 455 (M+1).

2-(3-{2-[{3-Bromo-4-[(diethoxy-phosphoryl)-difluoro-methyl]-benzyl}-(3,4-dichloro-phenyl)-amino]-oxazol-5-yl}-phenoxy)-benzoic acid methyl ester was prepared as per procedure D.

The title compound 2-(3-{2-[[3-Bromo-4-(difluoro-phosphono-methyl)-benzyl]-(3,4-dichloro-phenyl)amino]-oxazol-5-yl}-phenoxy)-benzoic acid methyl ester was prepared as described in procedure E.

¹H NMR (DMSO-d₆, 600 MHz): δ 3.71 (s, 3H), 5.29 (s, 2H), 6.76 (d, J=6.6 Hz, 1H), 7.07 (d, J=7.8 Hz, 1H), 7.10 (s, 1H), 7.27-7.62 (m, 10H), 7.85 (d, J=7.2 Hz, 1H), 7.93 (d, J=2.4 Hz, 1H); LCMS: m/z 754 (M+1).

### EXAMPLE 15

### 4′-{2-[[3-Bromo-4-(difluoro-phosphono-methyl)-benzyl]-(3,4-dichloro-phenyl)-amino]-oxazol-5-yl}-biphenyl-2-carboxylic acid methyl ester

4′-Acetyl-biphenyl-2-carboxylic acid methyl ester was prepared as described in procedure L.

4′-[2-(3,4-Dichloro-phenylamino)-oxazol-5-yl]-biphenyl-2-carboxylic acid methyl ester was prepared as described in procedure A-C as a yellow solid (3.62 g, 53% yield).

¹H NMR (CDCl₃, 600 MHz): δ 3.70 (s, 3H), 7.36-7.60 (m, 10H), 7.70 (d, J=1.8 Hz, 1H), 7.94 (d, J=6.6 Hz, 1H), 12.25 (bs, 1H); LCMS: m/z 440 (M+1).

Sodium hydride (0.21 g, 8.79 mmol) was suspended in 10 mL dry DMF. A solution of 4′-[2-(3,4-dichloro-phenylamino)-oxazol-5-yl]-biphenyl-2-carboxylic acid methyl ester in 10 mL DMF was added dropwise resulting in vigorous gas evolution. After stirring at room temperature for 15 min, [(2-bromo-4-bromomethyl-phenyl)-difluoro-methyl]-phosphonic acid diethyl ester (solution in 5 mL DMF) was added dropwise. After stirring at room temperature for 2 hours, the solvent was removed in vacuo. Saturated aqueous ammonium chloride was added and the mixture was extracted thrice with dichloromethane. The combined organic extracts were dried (MgSO₄) then concentrated in vacuo. Purification by silica gel flash chromatography gave 4′-{2-[{3-Bromo-4-[(diethoxy-phosphoryl)-difluoro-methyl]-benzyl}-(3,4-dichloro-phenyl)-amino]-oxazol-5-yl}-biphenyl-2-carboxylic acid methyl ester as a yellow solid (1.75 g, 38%).

¹H NMR (CDCl₃, 600 MHz): δ 1.32 (t, J=7.2 Hz, 6H), 3.68 (s, 3H), 4.23 (m, 4 H), 5.18 (s, 2H), 7.17-7.85 (m, 15H); LCMS: m/z 795 (M+1).

The title compound 4′-{2-[[3-Bromo-4-(difluoro-phosphono-methyl)-benzyl]-(3,4-dichloro-phenyl)-amino]-oxazol-5-yl}-biphenyl-2-carboxylic acid methyl ester was prepared as described in procedure E.

¹H NMR (DMSO-d₆, 600 MHz): δ 3.61 (s, 3H), 5.35 (s, 2H), 7.33 (d, J=8.4 Hz, 2H), 7.43-7.75 (m, 12H), 7.98 (d, J=2.4 Hz, 1H); LCMS: m/z 737 (M+1).

### EXAMPLE 16

### (3-{2-[[3-Bromo-4-(difluoro-phosphono-methyl)-benzyl]-(3,4-dichloro-phenyl)-amino]-oxazol-5-yl}-phenoxy)-phenyl-acetic acid

The title compound (3-{2-[[3-Bromo-4-(difluoro-phosphono-methyl)-benzyl]-(3,4-dichloro-phenyl)-amino]-oxazol-5-yl}-phenoxy)-phenyl-acetic acid was prepared from the compound of Example 13 using procedure F.

¹H NMR (600 MHz, CDCl₃) δ 7.97-6.90 (m, 16 H), 6.10 (s, 1H), 5.35 (s, 2H); LCMS m/z 755 [M+1].

### EXAMPLE 17

### 4′-{2-[[3-Bromo-4-(difluoro-phosphono-methyl)-benzyl]-(3,4-dichloro-phenyl)-amino]-oxazol-5-yl}-biphenyl-2-carboxylic acid

The title compound 4′-{2-[[3-Bromo-4-(difluoro-phosphono-methyl)-benzyl]-(3,4-dichloro-phenyl)-amino]-oxazol-5-yl}-biphenyl-2-carboxylic acid was prepared from the compound of Example 15 using procedure F.

¹H NMR (DMSO-d₆, 600 MHz): δ 5.35 (s, 2H), 7.36-7.74 (m, 14H), 7.98 (d, J=2.4 Hz, 1H); LCMS: m/z 723 (M−H)⁻, 361 (m−2H)²⁻.

### EXAMPLE 18

### 2-(3-{2-[[3-Bromo-4-(difluoro-phosphono-methyl)-benzyl]-(3,4-dichloro-phenyl)-amino]-oxazol-5-yl}-phenoxy)-benzoic acid

The title compound 2-(3-{2-[[3-Bromo-4-(difluoro-phosphono-methyl)-benzyl]-(3,4-dichloro-phenyl)-amino]-oxazol-5-yl}-phenoxy)-benzoic acid was prepared from the compound of Example 14 using procedure F.

¹H NMR (of the trilithium salt, D₂O, 600 MHz): δ 5.07 (s, 2H), 6.90-7.67 (m, 15H); LCMS: m/z 739 (M−H)⁻,369 (m−2H)²⁻.

### EXAMPLE 19

### 3′-{2-[[3-Bromo-4-(difluoro-phosphono-methyl)-benzyl]-(3,4-dichloro-phenyl)-amino]-oxazol-5-yl}-biphenyl-2-carboxylic acid methyl ester

The title compound was prepared in a manner analogous to that of the compound of Example 15 in 99% yield as a white solid.

¹H NMR (DMSO-d₆, 600 MHz): δ 3.52 (s, 3H), 5.29 (s, 2H), 7.13-7.92 (m, 15H); LCMS: m/z 737 (M+H)⁺.

### EXAMPLE 20

### 3′-{2-[[3-Bromo-4-(difluoro-phosphono-methyl)-benzyl]-(3,4-dichloro-phenyl)-amino]-oxazol-5-yl}-biphenyl-2-carboxylic acid

The title compound was prepared as described for the compound of Example 17 as an off-white solid in 71% yield.

¹H NMR (DMSO-d₆, 600 MHz): δ 5.32 (s, 2H), 7.19-7.99 (m, 15H); LCMS: m/z 723 (M−1).

### EXAMPLE 21

### 3-(3-{5-[[3- benzyl]-(3,4-dichloro-phenyl)-amino]-[1,3,4 ] oxadiazol-2-yl}-phenoxy)-benzoic acid

A mixture of 3-bromomethylbenzaldehyde (1.00 g, 5.40 mmol), methyl 3-hydroxybenzoate (0.987 g, 6.50 mmol) and potassium carbonate (1.49 g, 10.8 mmol) in dry pyridine (8.0 mL) is stirred under argon at room temperature. Copper (II) oxide (0.860 g, 10.8 mmol) is added and the reaction mixture is refluxed for 12 hours. After cooling to room temperature, CH₂Cl₂ (50 mL) is added and the mixture is filtered through celite. The filter cake is washed with fresh CH₂Cl₂ (50 mL). The combined organics are concentrated in vacuo. The residue is purified by flash chromatography (ethyl acetate/hexanes, 1:10 to 1:4) to yield methyl 3-(3-carbonylphenoxy) benzoate (0.776 g, 56%) as a yellow oil.

¹H NMR (300 MHz, CDCl₃): δ 9.97 (s, 1H), 7.85 (d, 1H, J=6.9 Hz), 7.69-7.24 (m, 7 H), 3.91 (s, 3H).

“Jones Reagent” was first prepared by adding concentrated H₂SO₄ (3.29 ml) dropwise to a solution of CrO₃ (3.84 g, 38.4 mmol) in water (11 mL) at 0° C. “Jones Reagent” was then added dropwise to a solution of 3-(3-Formyl-phenoxy)-benzoic acid methyl ester (8.86 g, 34.8 mmol) in acetone (36 ml) at 0° C. The reaction mixture was warmed to room temperature and stirred for 2.5 hours, after which isopropanol (21 mL) was added and the reaction mixture was stirred for an additional 12 hours. The reaction mixture was then filtered through celite and the filter cake was washed with fresh ethyl acetate (50 mL). The organic layers were concentrated in vacuo and the residue was dissolved in ethyl acetate (50 mL) and washed with 1× water (50 mL) then 1× brine (50 mL). The organic phase was dried over Na₂SO₄ and concentrated in vacuo to yield a yellow solid which was purified by triturating in hexanes to afford the desired product (7.86 g, 83%) as an off-white solid;

¹H NMR (300 MHz, DMSO-d₆): δ 13.18 (bs, 1H), 7.79-7.75 (m, 2H), 7.61-7.48 (m, 4H), 7.40-7.34 (m, 2H), 3,83 (s, 3H).

To 3-(3-methyl benzoate-phenoxy)-benzoic acid (2.00 g, 7.36 mmol.) in CH₂Cl₂ (30 mL) was added oxalyl chloride (1.28 ml, 14.7 mmol.) and DMF (0.10 mL, 1.3 mmol.). The resulting mixture was stirred at room temperature for 2 hours, after which it was concentrated in vacuo to give a yellow solid. With no further purification the crude solid was dissolved in CH₂Cl₂ (3.0 mL) and was added dropwise to a mixture of [(3,4-dichlorophenyl)amino]hydrazinomethane-1-thione (1.82 g, 7.73 mmol.) in pyridine (10 mL). The resulting reaction mixture was stirred for 19 hours at room temperature, after which it was concentrated in vacuo to a brown residue. The residue was dissolved in toluene (10 mL) and concentrated again in vacuo. With no further purification the resulting residue was dissolved in a benzene (50 mL) and acetone (2.0 mL) mixture and to the mixture was added EDC (1.40 g, 7.32 mmol). The reaction mixture was stirred for 6 hours at 90° C., after which it was partitioned between ethyl acetate and 1 N HCl. The aqueous layer was extracted with 3×100 mL ethyl acetate and the combined organic layers were washed with brine, dried over Na₂SO₄ and concentrated in vacuo to give an orange syrup which was purified by column chromatography (ethyl acetate:hexanes, 1:8 10 2:1) to yield a yellow solid. Further purification was performed by washing the solid in toluene and then 1:4 ethyl acetate:hexanes to yield the desired product, 3-{3-[5-(3,4-Dichloro-phenylamino)-[1,3,4]oxadiazol-2-yl]-phenoxy}-benzoic acid methyl ester (0.81 g, 24%) as a white solid.

¹H NMR (300 MHz, DMSO-d₆): δ 11.08 (s, 1H), 7.92-7.91 (m, 1H), 7.82-7.80 (m, 1H), 7.72-7.41 (m, 8H), 7.32-7.29 (m, 1H), 3.84 (s, 3H).

The title compound 3-(3-{5-[[3- benzyl]-(3,4-dichloro-phenyl)-amino]-[1,3,4]oxadiazol-2-yl}-phenoxy)-benzoic acid was prepared as described in procedure J and F.

¹H NMR (300 MHz, DMSO-d₆) δ 13.04 (bs, 2H), 7.972-7.966 (m, 1H), 7.90-7.87 (m, 2H), 7.78-7.77 (m, 1H), 7.69-7.43 (m, 9 H), 7.39-7.37 (m, 1H), 7.27-7.25 (m, 1H), 5.37 (s, 2H); LCMS m/z 574 [M⁻].

### EXAMPLE 22

### 3-[3-(5-{(3,4-Dichloro-phenyl)-[4-(difluoro-phosphono-methyl)-benzyl]-amino}-[1,3,4 ]oxadiazol-2-yl)-phenoxy]-benzoic acid methyl ester

3-{3-[5-((3,4-Dichloro-phenyl)-{4-[(diethoxy-phosphoryl)-difluoro-methyl]-benzyl}-amino)-[1,3,4]oxadiazol-2-yl]-phenoxy}-benzoic acid methyl ester was prepared as described for the compound of Example 21. Normal isomer (0.53 g, 77%) (1:2 ethyl acetate:hexanes, R_{f}=0.06).

¹H NMR (300 MHz, CDCl₃): δ 7.84-7.82 (m, 1H), 7.66 (s, 1H), 7.61-7.57 (m, 3H), 7.48-7.39 (m, 6H), 7.24-7.15 (m, 2H), 7.11-7.08 (m, 1H), 5.20 (s, 2H), 4.20-4.09 (m, 4H), 3.90 (s, 3H), 1.30-1.24 (m, 6H).

Iso compound (49.2 mg, 7.0%) (1:2 ethyl acetate:hexanes, R_{f}=0.15);

¹H NMR (300 MHz, CDCl₃): δ 7.85-7.83 (m, 1H), 7.67-7.52 (m, 7 H), 7.48-7.42 (m, 3H), 7.34-7.31 (m, 2H), 7.25-7.23 (m, 1H), 7.12-7.05 (m, 2H), 5.08 (s, 2H), 4.29-4.15 (m, 4 H), 3.90 (s, 3H), 1.42-1.26 (m, 6H).

The title compound 3-[3-(5-{(3,4-Dichloro-phenyl)-[4-(difluoro-phosphono-methyl)-benzyl]-amino}-[1,3,4]oxadiazol-2-yl)-phenoxy]-benzoic acid methyl ester was prepared as described in procedure E.

¹H NMR (300 MHz, DMSO-d₆) δ 8.00-7.99 (m, 1H), 7.78-7.76 (m, 1H), 7.65-7.37 (m, 12H), 7.27-7.25 (m, 1H), 5.35 (s, 2H).

### EXAMPLE 23

### 3-[3-(5-{(3,4-Dichloro-phenyl)-[4-(difluoro-phosphono-methyl)-benzyl]-amino}-[1,3,4]oxadiazol-2-yl)-pbenoxy]-benzoic acid

The title compound 3-[3-(5-{(3,4-Dichloro-phenyl)-[4-(difluoro-phosphono-methyl)-benzyl]-amino}-[1,3,4]oxadiazol-2-yl)-phenoxy]-benzoic acid was prepared as described in procedure F.

¹H NMR (300 MHz, DMSO-d₆) δ 8.00-7.99 (m, 1H), 7.78-7.76 (m, 1H), 7.65-7.37 (m, 12H), 7.27-7.25 (m, 1H), 5.35 (s, 2H).

### EXAMPLE 24

### 3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid methyl ester

The title compound 3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid methyl ester was prepared as described for the compound of Example 22.

¹H NMR (300 MHz, DMSO-d₆) δ 8.012-8.005 (m, 1H), 7.80-7.52 (m, 9 H), 7.45-7.40 (m, 3H), 7.27-7.25 (m, 1H), 5.33 (s, 2H), 3.84 (s, 3H).

### EXAMPLE 25

### 3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid

The title compound 3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid was synthesized as described for the compound of Example 23.

¹H NMR (300 MHz, DMSO-d₆) δ 8.02-8.01 (m, 1H), 7.78-7.74 (m, 3H), 7.67-7.54 (m, 6H), 7.50-7.44 (m, 2H), 7.39-7.37 (m, 1H), 7.28-7.25 (m, 1H), 5.34 (s, 2H).

### EXAMPLE 26

### 4′-{2-[[3-Bromo-4-(difluoro-phosphono-methyl)-benzyl]-methyl-amino]-oxazol-5-yl}-biphenyl-2-carboxylic acid

4′-{2-[{3-Bromo-4-[(diethoxy-phosphoryl)-difluoro-methyl]-benzyl}-methyl-amino]-oxazol-5-yl}-biphenyl-2-carboxylic acid (210 mg, 72%) was prepared as described in Example 17. ¹H NMR of the trisodium salt (D₂O, 600 MHz): δ 3.06 (s, 3H), 4.63 (s, 2H), 7.15 (s, 1H), 7.36-7.55 (m, 9H), 7.65 (s, 1H), 7.74 (d, J=7.8 Hz, 1H); MS (ESI): m/z 592 (M−H)⁻.

### EXAMPLE 27

### 4′-{2-[[3-Bromo-4-(difluoro-phosphono-methyl)-benzyl]-phenyl-amino]-oxazol-5-yl}-biphenyl-2-carboxylic acid

The title compound was prepared as described in Example 17 (371 mg, 60%). ¹H NMR of the trisodium salt (D₂O, 600 MHz): δ 5.08 (s, 2H), 7.13 (s, 1H), 7.26-7.48 (m, 14H), 7.63 (s, 2H); MS (ESI): m/z 654 (M−H)⁻, 326 (m−2H)² .

### EXAMPLE 28

### 4′-{2-[[3-Bromo-4-(difluoro-phosphono-methyl)-benzyl]-(4-fluorophenyl)-amino]-oxazol-5-yl}-biphenyl-2-carboxylic acid

The title compound was prepared as described in Example 17. (238 mg, 35%). ¹H NMR of the trisodium salt (D₂O, 600 MHz): δ 5.08 (s, 2H), 7.11-7.15 (m, 3H), 7.37-7.65 (m, 13H); MS (ESI): m/z 672 (M−H)⁻, 335 (m−2H)²⁻.

### EXAMPLE 29

### 4′-{2-[[3-Bromo-4-(difluoro-phosphono-methyl)-benzyl]-(4-chlorophenyl)-amino]-oxazol-5-yl}-biphenyl-2-carboxylic acid

The title compound was prepared as described in Example 17 (379 mg, 63%). ¹H NMR of the trisodium salt (D₂O, 600 MHz): δ 5.07 (s, 2H), 7.14 (s, 1H), 7.33-7.61 (m, 15 H); MS (ESI): m/z 688 (M−H)⁻, 343 (m−2H)²⁻.

### EXAMPLE 30

### 4′-{2-[[3-Chloro-4-(difluoro-phosphono-methyl)-benzyl]-(4-chlorophenyl)-amino]-oxazol-5-yl}-biphenyl-2-carboxylic acid

The title compound was prepared as described in Example 17 (40 mg, 22%). ¹H NMR of the trisodium salt (D₂O, 600 MHz): δ 5.17 (s, 2H), 7.22 (s, 1H), 7.34-7.67 (m, 15H); MS (ESI): m/z 644 (M−H)⁻, 321 (m−2H)²⁻.

### EXAMPLE 31

### {[2-Chloro-4-({(3,4-dichloro-phenyl)-[5-(4-methanesulfonyl-phenyl)-oxazol-2-yl]-amino}-methyl)-phenyl]-difluoro-methyl}-phosphonic acid

The title compound was prepared as described in Example 3: mp: 141-143° C.; ¹H NMR (600 MHz, DMSO-d₆) δ 7.99 (s, 1H), 7.91 (d, 2H), 7.74-7.77 (overlapping, 4H), 7.63 (d, 1H), 7.53 (d, 1H), 7.37 (s, 1H), 7.26 (d, 1H), 7.19 (bs, 2H), 5.32 (s, 2H), 3.21 (s, 3H); Mass: 637, 639 (M+1), 635, 637 (M−1).

### EXAMPLE 32

### {[2-Bromo-4-({(4-fluoro-phenyl)-[5-(4-methanesulfonyl-phenyl)-oxazol-2-yl]-amino}-methyl)-phenyl]-difluoro-methyl}-phosphonic acid

The title compound was prepared as described in Example 3. ¹H NMR (CDCl₃, 600 MHz): δ 3.19 (s, 3H), 5.25 (s, 2H), 7.26-7.90 (m, 12 H); MS (ESI): m/z 630 (M−H)⁺.

### EXAMPLE 33

### {[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid mono-(1-isopropoxycarbonyloxy-ethyl) ester

To a solution of {[2-Bromo-4-({(3,4-dichloro-phenyl)-[5-(4-methanesulfonyl-phenyl) oxazol-2-yl]-amino}-methyl)-phenyl]-difluoro-methyl}-phosphonic acid (341 mg, 0.5 mmol) in 4 mL N,N-dimethylformamide under nitrogen atmosphere was added diisoprop yl ethylamine (0.174 mL, 1 mmol) was added followed by 1-chloroethyl isopropyl carbonate (prepared according to the procedure in EP 0 682 023). The mixture was stirred at 40° C. for 14 hours. Tetra-n-butyl ammonium iodide (19 mg, 10 mol %) was added to the mixture and continued heating at 40° C. for another 72 hours. Solvent was evaporated under reduced pressure. The residue was dissolved in 30 mL dichloromethane and washed with 20 Ml water followed by 20 mL brine, dried over anhydrous sodium sulphate, filtered and evaporated under reduced pressure. The residue was purified on silica gel column using hexane (400 mL), 1:1 hexane: ethyl acetate (500 mL), dichloromethane (400 mL) and 20:1 dichloromethane: ethanol (600 mL) and 10:1 dichloromethane: ethanol (400 mL) to elute the compound. Fractions containing pure compound were combined and evaporated to obtain 112 mg (27%) of title compound.

¹H NMR (DMSO-d₆, 600 MHz): δ 1.18 (s, 6H), 1.27 (s, 3H), 3.20 (s, 3H), 4.71 (q, J=6 Hz, 1H), 5.32 (s, 2H), 6.16 (s, 1H), 7.33-7.98 (m, 12H); MS (ESI): m/z 811 (M−H)⁺.

The remaining compounds shown in Tables 1 and 2 were prepared using the procedure as for the compounds shown above, using appropriate starting materials.

### EXAMPLE 34

### {[2-Bromo-4-({(3,4-dichloro-phenyl)-[5-(4-methanesulfonyl-phenyl)-oxaz ol-2-yl]-amino}-methyl)-phenyl]-difluoro-methyl}-phosphonic acid mono-(L-alanyl ethyl ester) amidate

To a solution of {[2-Bromo-4-({(3,4-dichloro-phenyl)-[5-(4-methanesulfonylphenyl) oxazol-2-yl]-amino}-methyl)-phenyl]-difluoro-methyl}-phosphonic acid (852 mg, 1.25 mmol) in 15 mL anhydrous N,N-dimethylformamide was added diisopropyl ethyl amine (0.695 mL, 4 mmol) followed by dicyclohexyl carbodiimide (387 mg, 1.87 mmol). To this solution was added L-alanine ethyl ester hydrochloride (230 mg, 1.5 mmol) and the mixture was stirred at 40° C. for 14 hours. The mixture was cooled to room temperature and filtered. The solvent was removed under reduced pressure. The product was purified on a reverse phase (C18) column using acetonitrile/water (gradient 10%, 20% and 30%). The fractions were analyzed, pooled and solvent was removed under reduced pressure. The product obtained was dissolved in 10 mL water and acidified to pH 2. The precipitate formed was extracted into ethyl acetate (30 mL). The ethyl acetate layer was washed with water (10 mL), brine (10 mL), dried over anhydrous sodium sulphate, filtered and evaporated to get 110 mg (11.3%).

¹H NMR (DMSO-d₆, 600 MHz): δ (ppm) 1.15 (m, 6H), 3.20 (s, 3H), 3.67 (m, 1H), 4.02 (q, 2H), 5.35 (s, 2H), 7.39-7.98 (m, 11H); MS (ESI): m/z 780 (M−H)⁺.

### EXAMPLE 35

### 2,2-Dimethyl-propionic acid {[2-bromo-4-({(4-fluoro-phenyl)-[5-(4-methanesulfonylphenyl)-oxazol-2-yl]-amino}-methyl)-phenyl]-difluoro-methyl}-(2,2-dimethyl-propionyloxymethoxy)-phosphinoyloxymethyl ester

{[2-Bromo-4-({(4-fluoro-phenyl)-[5-(4-methanesulfonyl-phenyl)-oxazol-2-yl]-amino}-methyl)-phenyl]-difluoro-methyl}-phosphonic acid (100 mg, 0.159 mmol) was dissolved in 3.19 mL 0.0996M NaOH (0.318 mmol, 2.0 eq.). To this solution was added silver(I) nitrate (67 mg, 0.397 mmol) as a solution in 0.5 mL water. The resulting fine precipitate was collected by filtration then rinsed with water, ethanol, then ether and dried in vacuo to give 106 mg (79%) of the bis-silver salt.

The silver salt was suspended in 1 mL dry toluene. lodomethyl pivalate (91 mg, 0.38 mmol) was added (exothermic). The suspension was stirred at room temperature for 4.5 h then loaded directly onto a silica gel column and eluted with 50% EtOAc/hexanes to give the title compound as a white solid (56 mg, 52% from silver salt). ¹H NMR (CDCl₃, 600 MHz): δ (ppm) 1.22 (s, 18H), 3.05 (s, 3H), 5.12 (s, 2H), 5.69 (dd, J=12.0 Hz, 4.8 Hz, 2H), 5.75 (dd, J=12.0 Hz, 4.8 Hz, 2H), 7.10 (m, 2H), 7.28-7.36 (m, 4 H), 7.53 (d, J=9.0 Hz, 2H), 7.57 (d, J=7.8 Hz, 1H), 7.67 (s, 1H), 7.88 (d, J=9.0 Hz, 2H); MS (ESI): m/z 861 (M+H)⁺.

### EXAMPLE 36

### {[2-Bromo-4-({(4-fluoro-phenyl)-[5-(4-methanesulfonyl-phenyl)-oxazol-2-yl]-amino}methyl)-phenyl]-difluoro-methyl}-phosphonic acid

The title compound was prepared as described in procedure A-E.

¹H NMR (300 MHz, DMSO-d₆) δ 7.26-7.90 (m, 12H), 5.25 (s, 2H), 3.19 (s, 3H); MS (ESI): m/z 630 (M−H)+.

### EXAMPLE 37

### [(2-Bromo-4-{[[5-(4-carbamoyl-phenyl)-oxazol-2-yl]-(4-fluoro-phenyl)-amino]-methyl}-phenyl)-difluoro-methyl]-phosphonic acid

4-(2-Bromo-acetyl)-benzonitrile was prepared from 4-acetyl benzonitrile and bromine using procedure A.

4-(2-Azido-acetyl)-benzonitrile was prepared from 4-(2-bromo-acetyl)-benzonitrile as in procedure B.

4-[2-(4-Fluoro-phenylamino)-oxazol-5-yl]-benzonitrile obtained as an off-white solid using 4-(2-Azido-acetyl)- benzonitrile and fluoro-4-isothiocyanato-benzene according to procedure C.

[(2-Bromo-4-{[[5-(4-cyano-phenyl)-oxazol-2-yl]-(4-fluoro-phenyl) amino]-methyl}-phenyl)-difluoro-methyl]-phosphonic acid diethyl ester was prepared as a light yellow solid from 4-[2-(4-Fluoro-phenylamino)-oxazol-5-yl]-benzonitrile and [(2-bromo-4-bromomethyl-phenyl)-difluoro-methyl]-phosphonic acid diethyl ester as in procedure D. To a solution of [(2-Bromo-4-{[[5-(4-cyano-phenyl)-oxazol-2-yl]-(4-fluoro-phenyl) amino]-methyl}-phenyl)-difluoro-methyl]-phosphonic acid diethyl ester (200 mg, 0.31 mmol) in 3 ml dimethylsulfoxide cooled in ice-bath was added 100 mg potassium carbonate followed by 2 ml hydrogen peroxide (30% v/v in water). The mixture was stirred for 30 minutes and then diluted with ethyl acetate. Washed with 2% sodium dithionite in water, 1N HCl and water. The ethyl acetate solution was dried over anhydrous sodium sulfate, filtered and evaporated. The residue was purified on a silica gel column to give [(2-Bromo-4-{[[5-(4-carbamoyl-phenyl)-oxazol-2-yl]-(4-fluoro-phenyl)-amino]-methyl}-phenyl)-difluoro-methyl]-phosphonic acid diethyl ester. Deprotection of [(2-bromo-4-{[[5-(4-carbamoyl-phenyl)-oxazol-2-yl]-(4-fluoro-phenyl)-amino]-methyl}-phenyl)-difluoro -methyl]-phosphonic acid diethyl ester was done using bistrimethylsilyltrifluoro- acetamide as per procedure E to obtain the title compound [(2-Bromo-4-{[[5-(4-carbamoyl-phenyl)-oxazol-2-yl]-(4-fluoro-phenyl)-amino]-methyl}-phenyl)-difluoro-methyl]-phosphonic acid.

### EXAMPLE 38

### ({2-Bromo-4-[((4-fluoro-phenyl)-{5-[4-(2H-tetrazol-5-yl)-phenyl]-oxazol-²-yl}-amino)-methyl]-phenyl}-difluoro-methyl)-phosphonic acid

[(2-Bromo-4-{[[5-(4-cyano-phenyl)-oxazol-2-yl]-(4-fluoro-phenyl)-amino]-methyl}-phenyl)-difluoro-methyl]-phosphonic acid was prepared by deprotection of [(2-Bromo-4-{[[5-(4-cyano-phenyl)-oxazol-2-yl]-(4-fluoro-phenyl) amino]-methyl}-phenyl)-difluoro-methyl]-phosphonic acid diethyl ester (intermediate in previous compound, SBI-34746) as per procedure E.

To a solution of [(2-Bromo-4-{[[5-(4-cyano-phenyl)-oxazol-2-yl]-(4-fluoro-phenyl)-amino]-methyl}-phenyl)-difluoro-methyl]-phosphonic acid (225 mg, 0.39 mmol) in anhydrous N,N-dimethylformamide (5 ml) was added ammonium chloride (212 mg, 4 mmol) and sodium azide (265 mg, 3.9 mmol). The mixture was heated at 115° C. for 24 h and cooled to room temperature. An additional 112 mg of ammonium chloride and 113 mg of sodium azide were added and heated at 115° C. for 24 hours. The reaction was cooled to room temperature and diluted with ethyl acetate and washed with 1N HCl, organic solution dried over anhydrous sodium sulfate, filtered and concentrated. The crude product was purified on C₁₈ reverse phase column (5% CH₃CN: H₂O to 95% CH₃CN: H₂O) to get 75 mg (31%) of the title product.

### EXAMPLE 39

Assay Methods

A 5× stock of pNPP (p-nitrophenol phosphate) substrate is prepared as 50 mM pNPP in assay buffer. Various tyrosine phosphatase solutions can be prepared as follows:
PTP-1B (purified, 1 mg/mL) as a 1:250 dilution (to a final concentration of 4:g/mL);
TC-PTP (NEB, 1000 units in 100:L) as a 1:50 dilution (to a final concentration of 2 U/10:L (4:g/mL));
CD45 (Calbiochem, 20:g, 400 units in 100: L) as a 1 :50 dilution (to a final concentration of 0.8 U/10:L (4:g/mL));
LAR (NEB, 1000 units in 200:L) as a 1:75 dilution (to a final concentration of 0.7 U/10:L (4:g/mL)); and
PTP-β (UBI, #14-350, 10,000 units, 40:g/571:L) as a 1:17.5 dilution (to a final concentration of 10 U/10:L (4:g/mL));

The compound to be tested is prepared as 1:16.7 and 1:50 dilutions from stock in a total volume of 1 00:M DMSO to give final concentrations of 626 and 200:M. The reaction mixtures are prepared in a 96-well microtiter plate (on ice) as 55:L assay buffer, 5:L of the diluted compound (to a final concentration of 31.3 and 10:M), 20:L of the pNPP substrate solution (to a final concentration of 10 mM) and 20:L PTPase in assay buffer. The reactants are mixed well, the plate placed in a water bath at 30° C. and incubated for 10 minutes. The reaction is then terminated by adding 1 OO:L of 2M K₂CO3 per well, and the absorbance is measured at 405 nm by conventional means.

Unless otherwise indicated, this assay was used to determine activity for the selected compounds whose activity is recorded in Table 1 and Table 2.

Since modifications will be apparent to those of skill in the art, the subject matter claimed herein is intended to be limited only to the scope of the appended claims.

What is claimed is:

1. A compound having the formula: 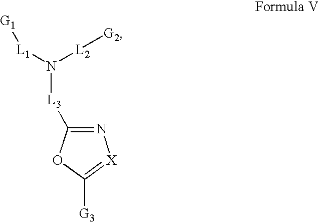
or a pharmaceutically acceptable salt thereof, wherein:
L₁, L₂ , and L₃ are each, independently, a bond or (CH₂), where s is 1-3;
X is CR7 or N, wherein R7 is H or C1-C3 alkyl;
G₁ is H or a phenyl ring, wherein the phenyl ring is optionally substituted with one or more moieties selected from the group consisting of: phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxyalkyl ester, where acyl is C₂-C₇ alkanoyl or C4-C7 cycloalkanoyl, phosphonodifluoromethyl mono-alkoxycarbonyloxyalkyl ester, where alkoxy is C1-C6 or C3-C6 cycloalkoxy, phosphonodifluoromethyl mono-alkoxyalkyl ester, where alkoxy is C15-C22, phosphonodifluoromethyl mono-amide, 2-carboxyethenyl optionally substituted with 1-2 fluorines or methyl groups, carboxymethoxy, carboxy —C2-C4-alkyl optionally further substituted with 1-4 halogen atoms or 1-4 methyl groups, Cl, Br, F, CN, OH, CH₃ , and ethynyl;
G₂ is H, C₁-C₃ alkyl, or a phenyl or pyridyl ring, wherein the alkyl, phenyl or pyridyl group is optionally and independently substituted with 1, 2, or 3 of the following moieties: Cl, F, Br, carboxy, methoxycarbonyl, OCH₃, OCF₃, OCHF₂, C₁-C₃ alkyl, and C₁-C₃-alkylsulfonyl;
G₃ is H, C₁-C₃ alkyl, or a phenyl or pyridyl ring, wherein the phenyl or pyridyl ring is optionally substituted with:
(i) F, Cl, Br, CF₃, OR, methoxycarbonyl, carboxy, (CRR₁)ₙCO₂R, CF₂CO₂R, O(CRR₁)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR₁)CO₂R, S(O)ₘ(CRR₁)CONRR1, SO₂NRR1, C₁-C₃-alkylsulfonyl, CF₂P(═O)(OR)(OR1);
(ii) phenyl, wherein said phenyl is optionally further substituted with F, Cl, Br, CF₃, OR, methoxycarbonyl, carboxy, (CRR1)ₙCO₂R, CH₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, S(O)ₘ(CRR1)CONRR1, SO₂NRR1, C₁-C₃-alkylsulfonyl, or CF₂P(═O)(OR)(OR1);
(iii) phenoxy, wherein said phenoxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, S(O)ₘ(CRR₁)CONRR1, SO₂NRR1, C₁-C₃-alkylsulfonyl, or CF₂P(═O)(OR)(OR1); or
(iv) benzyloxy, wherein said benzyloxy is optionally further substituted with F, Cl, Br, CF₃, OR, methoxycarbonyl, carboxy, (CRR1)ₙCO₂R, CF₂CO₂R, O(CRR1)CO₂R, CH═CHCO₂R, tetrazolyl (Tzl), NRR1, NRC(═O)OR1, OC(═O)NRR1, C(═O)NRR1, NRC(═O)C(═O)OR1, SO₂NRR1, S(O)ₘ(CRR1)CO₂R, S(O)ₘ(CRR₁)CONRR1, SO₂NRR1, C₁-C₃-alkylsulfonyl, or CF₂P(═O)(OR)(OR1);
wherein m=0 to 6 and n=0 to 2;
wherein R and R1 are independently selected from hydrogen, an alkyl group of 1 to 6 carbon atoms, wherein the alkyl group is unsubstituted or mono-, di- or tri-substituted with 1 to 3 substituents selected from the group consisting of Y₁ , Y₂ , and Y₃ , an aryl group, wherein the aryl group is unsubstituted or mono-, di- or tri- substituted with 1 to 3 substituents selected from the group consisting of Y₁, Y₂ , and Y₃ , —OC(R2 R3)OC(═O)R4, and —OC(R2 R3)OC(═O)OR4, or R and R1 are joined to form a 4-8 membered cycloalkyl, cycloalkenyl, cycloalkynyl, or heterocyclic ring;
wherein R2, R3 and R4 are independently selected from (i) and (ii) as follows:
(i) H, C₁-C₇ alkyl, alkenyl of 2 to 6 carbon atoms, wherein the alkenyl group is unsubstituted or mono-, di- or tri-substituted with 1 to 3 substituents selected from the group consisting of Y₁, Y₂ , and Y₃ , alkynyl of 2 to 6 carbon atoms, wherein the alkynyl group is unsubstituted or mono-, di- or tri-substituted with 1 to 3 substituents selected from the group consisting of Y₁, Y₂ , and Y₃ , cycloalkyl of 3 to 8 carbon atoms, wherein the cycloalkyl group is unsubstituted or mono-, di- or tri-substituted with 1 to 3 substituents selected from the group consisting of Y₁, Y₂ , and Y₃ , aryl of 6 to 14 carbon atoms, wherein the aryl group is unsubstituted or mono-, di- or tri-substituted with 1 to 3 substituents selected from the group consisting of Y₁, Y₂ , and Y₃ , linked biaryl or heterobiaryl groups of 10 to 20 atoms featuring two aromatic or heteroaromatic ring systems linked through a single bond, with the ring atoms selected from carbon and heteroatoms, wherein the heteroatoms are selected from oxygen, nitrogen, and sulfur, and wherein the linked biaryl or heterobiaryl group is unsubstituted or mono-, di- or tri-substituted with 1 to 3 substituents selected from the group consisting of Y₁, Y₂ , and Y₃ , aralkyl of 7 to 16 carbon atoms, wherein the aralkyl is unsubstituted or mono-, di- or tri-substituted with 1 to 3 substituents selected from the group consisting of Y₁, Y₂ , and Y₃ , monocyclic-heteroaryl or bicyclic-heteroaryl having 5 to 14 ring atoms with the ring atoms selected from carbon and heteroatoms, wherein the heteroatoms are selected from oxygen, nitrogen, and sulfur, and wherein the monocyclic-heteroaryl or bicyclic heteroaryl group is unsubstituted or mono-, di- or tri-substituted with 1 to 3 substituents selected from the group consisting of Y₁, Y₂ , and Y₃ , and a heteroaralkyl group of 5 to 14 ring atoms with the ring atoms selected from carbon and heteroatoms, wherein the heteroatoms are selected from oxygen, nitrogen, and sulfur, wherein the heteroaralkyl is unsubstituted or substituted on the alkyl chain and which is unsubstituted on the ring or mono-, di- or tri-substituted on the ring with 1 to 3 substituents selected from the group consisting of Y₁, Y₂ , and Y₃ ; or
(ii) R2 and R3, and/or R3 and R4, and/or R2 and R4 are joined to form a 4-8-membered cycloalkyl, cycloalkenyl, cycloalkynyl, or heterocyclic ring, and the other of R2, R3, and R4, when not joined in a ring, is selected as in (i) above;
and wherein Y1, Y2, and Y3 are each, independently, selected from (i) or (ii) as follows:
(i) R5, (CR5 R6)ₙOR5, OH, (CR5 R6)ₙNR5R6, C(=NR5)NR5R6, C(═NOR5)NR5R6, halogen (F, Cl, Br, I), cyano, nitro, CF₃, CF₂CF₃, CH₂CF₃, CH(CF₃)₂, C(OH)(CF₃)₂, OCHCl₂, OCF₃, OCF₂H, OCF₂CF₃, OCH₂CF₃, (CR5R6)ₙOC(═O)NR5R6, (CR5R6)ₙNHC(═O)C(═O)OR5, (CR5R6)ₙNHC(═O)NR5SO₂(Me, CF₃), (CR5R6)ₙNHSO₂(Me, CF₃), (CR5R6)ₙNHSO₂NR5R6, NHSO₂NR5C(═O)(Me, CF₃), (CR5R6)ₙNHC(═O)R5, (CR5R6)ₙNHC(═O)NR5R6, C(═O)OH, (CR5R6)ₙC(═O)OH, C(═O)OR5, C(═O)O(CR5R6)OC(═O)R5, C(═O)O(CR5R6)OC(═O)OR5, C(═O)R5,—(CR5R6)ₙC(═O)R5, (CF₂)ₙC(═O)R5, (CFR5) nC(═O)R5, tetrazolyl (Tzl), (CR5R6)ₙTzl, (CF₂)ₙTzl, (CFR5)ₙTzl, (CR5R6)ₙC(═O)OR5, (CR5R6)ₙC(═O)NH₂, (CR5R6)ₙC(═O)NR5R6, (CR5R6)ₙC(═O)C(═O)OR5, (CR5R6)ₙCH(OR5)C(═O)OR5, (CF₂)ₙC(═O)OH, (CF₂)ₙC(═O)OR5, (CF₂)ₙC(═O)NH₂, (CF₂)ₙC(═O)NR5R6, 017121-025999 (CR5R6)ₙC(═O)C(═O)OR5, (CR5R6)ₙCH(OR5)C(═O)OR5, C(R5)=C(R6), C(═O)OR5, C(R5)=C(R6)-Tzl, (CR5R6)ₙP(═O)(OH)₂, (CR5R6)ₙP(═O)(OR5)(OR6), P(═O)(OR5)[(OCR5R6)OC(═O)R5], P(═O)(OR5)[(OCR5R6)OC(═O)OR5], P(═O) [(OCR5R6)OC(═O)R5)] [(OCR5R6)OC(═O)R5], P(═O)[(OCR5R6)OC(═O)OR5)] [(OCR5R6)OC(═O)OR5], (CR5R6)ₙP(═O)(Me)(OR5), (CR5R6)ₙP(═O)(CF₃)(OR5), (CF₂)ₙP(═O)(OR5)(OR6), (CF₂)ₙP(═O)(Me)(OR5), (CF₂)ₙP(═O)(CF₃)(OR5), (CFR5)ₙP(═O)(OR5)(OR6), CR5═CR5-P(═O)(OR5)(OR6), CR5═CR5-P(═O)(Me)(OR5), CC-P(═O)(OR5)(OR6), (C═O)P(═O)(OR5)(OR6), (C═O)P(═O)(Me)(OR5), (C═O)P(═O)(CF₃)(0 R5), (CR5 OR6)ₙP(═O)(OR5)(OR6), (CR5 OR6)ₙP(═O)(Me)(OR5), (CR5 OR6)ₙP(═O)(CF₃)(OR5), O(CR5R6)ₙC(═O)OR5, O(CF₂)ₙC(═O)OR5, OC(═O)R5, OCH[C(═O)OR5]₂, O(CR5R6),CH[C(═O)OR5]₂, OCF [C(═O)OR5]₂, O(CR5R6)ₙC(═O)C(═O)OR5, O(CF₂)ₙC(═O)C(═O)OR5, O(CR5R6)ₙTzl, O(CF₂)ₙTzl, OCH(Tzl)₂, O(CF₂)ₙP(═O)(OR5)(OR6), O(CF₂)ₙP(═O)(Me)(OR5), O(CF₂)ₙP(═O)(CF₃)(OR5), O(CFR5)ₙP(═O)(OR5)(OR6), O(CFR5)ₙP(═O)(Me)(OR5), O(CFR5)ₙP(═O)(CF₃)(OR5), (CR5R6)ₙP(═O)(OR5)(OR6), O(CR5R6)ₙP(═O)(Me)(OR5), O(CR5R6)ₙP(═O)(CF₃)(OR5), OCF [P(═O)(Me)(OR5)]₂, SO₃ H, —(CR5R6)ₙSO₃ H, S(O), R5, SCF₃, SCHF₂, SO₂ CF₃, SO₂ Ph, (CR5R6)ₙS(O)ₙR5, (CR5R6)nS (0)₂ CF₃, (CR5R6)ₙSO₂ NR5R6, (CR5R6)ₙSO₂ NR5 C(═O)(Me, CF₃), (CF₂)ₙSO₃ H, (CFR5)ₙSO₃ H, and (CF₂)ₙSO₂ NR5R6, wherein n=0-2, and wherein R5 and R6 are each independently H, C1-C6 alkyl, C2-C6 alkenyl, C2-C6 alkynyl, a C3-C8 cycloalkyl ring, or a 5-7 membered heterocyclic ring; or
(ii) Y₁ and Y₂, and/or Y₁ and Y₃ , and/or Y₂ and Y₃ are selected together to be (CR5R6)₂₋₆, —O[C(R8)(R9)]ᵣO— or —O[C(R8)(R9)]ᵣ₊₁—, wherein r is an integer from 1 to 4 and R8 and R9 are independently selected from the group consisting of hydrogen, alkyl of 1 to 12 carbon atoms, aryl of 6 to 14 carbon atoms, heteroaryl of 5 to 14 ring atoms, aralkyl of 7 to 15 carbon atoms, and heteroarylalkyl of 5 to 14 ring atoms, and the other of Y1, Y2, and Y3, when not selected as in (ii), is selected as in (i) above.

2. The compound of claim 1, wherein X is CR7, and wherein R7 is H.

3. The compound of claim 1, wherein L₁ is CH₂.

4. The compound of claim 1, wherein L₃ is a bond.

5. The compound of claim 1, wherein G₁ is said optionally substituted phenyl ring.

6. The compound of claim 1, wherein G₁ is said phenyl ring substituted at the 3 position, the 4 position, or at both the 3 and 4 positions.

7. The compound of claim 1, wherein G₁ is said phenyl ring substituted with one or more of the following moieties: phosphonodifluoromethyl, phosphonodifluoromethyl monoethyl ester, phosphonodifluoromethyl monomethyl ester, phosphonodifluoromethyl diethyl ester, phosphonodifluoromethyl mono-acyloxymethyl ester or phosphonodifluoromethyl mono-acyloxyethyl ester, where acyl is C₂-C₇ alkanoyl or C4-C7 cycloalkanoyl, phosphonodifluoromethyl mono-alkoxycarbonyloxymethyl ester or phosphonodifluoromethyl mono-alkoxycarbonyloxyethyl ester, where alkoxy is C1-C6 or C3-C6 cycloalkoxy, phosphonodifluoromethyl mono-alkoxyalkyl ester, where alkoxy is C15-C22, phosphonodifluoromethyl mono-(alanyl ethyl ester)amide, 2-carboxyethenyl, carboxymethoxy, carboxy-C2-C4-alkyl, Cl, Br, and F.

8. The compound of claim 1, wherein G₁ is selected from the group consisting of: 4-(difluoro-phosphono-methyl)-3-bromo-benzyl, (4-{4-[(diethoxy-phosphoryl)-difluoro-methyl]-3-bromo-phenyl, 3-bromo-4-carboxymethoxy-benzyl, (4-{4-[(ethoxy-hydroxy-phosphoryl)-difluoro-methyl]-3-bromo-benzyl, 3-bromo-4-(2-carboxyvinyl)-benzyl, 4-(carboxy-difluoro-methyl)-benzyl, 4-{[(2,2-dimethyl-propionyloxymethoxy)-hydroxy-phosphoryl]-difluoro-methyl-3-bromo-benzyl, 4-(difluoro-(methoxy-hydroxy-phosphoryl)-methyl-3-bromo-benzyl, 4-(difluoro-phosphono-methyl)-benzyl, 4-carboxybenzyl, 4-(difluoro-phosphono-methyl)-3-chloro-benzyl, 4-(difluroro-(mono-((1-ethoxycarbonyl-1-ethyl)amido)-hydroxy-phosphoryl)methyl)-3-bromobenzyl, 4-(difluoro-(3-hexadecyloxy-propoxy)-hydroxy-phosphoryl)methyl)-3-bromobenzyl and 4-(difluoro-((1-isopropoxycarbonyloxy)ethoxy-hydroxy-phosphoryl)methyl)-3-bromo-benzyl.

9. The compound of claim 1, wherein G₁ is selected from the group consisting of: 4-(difluoro-phosphono-methyl)-3-bromo-phenyl, (4-{4-[(diethoxy-phosphoryl)-difluoro-methyl]-3-bromo-phenyl, 3-bromo-4-carboxymethoxy-phenyl, (4-{4-[(ethoxy-phosphoryl)-difluoro-methyl]-3-bromo-phenyl, 3-bromo-4-(2-carboxyvinyl)-phenyl, 4-(carboxy-difluoro-methyl)-phenyl, 4-{[(2,2-dimethyl-propionyloxymethoxy)-hydroxy-phosphoryl]-difluoro-methyl-3-bromo-phenyl, 4-(difluoro-(methoxy-hydroxy-phosphoryl)-methyl-3-bromo-phenyl, 4-(difluoro-phosphono-methyl)-phenyl, 4-carboxyphenyl, 4-(difluoro-phosphono-methyl)-3-chloro-phenyl, 4-(difluoro-(3-hexadecyloxy-propoxy)-hydroxy-phosphoryl)methyl)-3-bromobenzyl, 4-(difluroro-(mono-((1-ethoxycarbonyl-1-ethyl)amido)-hydroxy-phosphoryl)methyl)-3-bromophenyl and 4-(difluoro-((1-isopropoxycarbonyloxy)ethoxy-hydroxy-phosphoryl)methyl)-3-bromo-phenyl.

10. The compound of claim 1, wherein G₁ is said phenyl ring substituted with phosphonodifluoromethyl.

11. The compound of claim 1, wherein G2 is said phenyl ring substituted at the 3 position, the 4 position, or at both the 3 and 4 positions.

12. The compound of claim 1, wherein G₂ is selected from the group consisting methyl, phenyl, 4-fluorophenyl, 4-chlorophenyl, 3,4-dichlorophenyl, 4-methoxycarbonyl-benzyl, and 4-carboxybenzyl.

13. The compound of claim 1, wherein G₃ is said optionally substituted phenyl ring.

14. The compound of claim 1, wherein G₃ is selected from the group consistingof phenyl, 4-methoxycarbonylphenyl, 4-carboxyphenyl, 4-aminocarbonylphenyl, 4-methylsulfonylphenyl, 4-(4′-methoxycarbonyl-phenoxy)-phenyl, 4-(4′-carboxyphenoxy)phenyl, 3-(α-methoxycarbonylbenzyloxy)phenyl, 3-(2′-methoxycarbonyl-phenoxy)-phenyl, 4-(2′-methoxycarbonyl-phenyl)-phenyl, 3-(α-carboxybenzyloxy)phenyl, 3-(2′-carboxyphenoxy)phenyl, 3-(2′-methoxycarbonyl-phenyl)-phenyl, 3-(2′-carboxy-phenyl)-phenyl, 3-phenoxy-phenyl, 3-(3′-carboxyphenoxy)phenyl, 3-(3′-methoxycarbonylphenoxy)phenyl, 4-(aminocarbonylmethyl)thiophenyl, and 4-(2′-carboxyphenyl)phenyl.

15. The compound of claim 1, selected from:
[(2-Bromo-4-{[(3,4-dichloro-phenyl)-(5-phenyl-oxazol-2-yl)-amino]-methyl}-phenyl)-difluoro-methyl]-phosphonic acid
4-{2-[[3-Bromo-4-(difluoro-phosphono-methyl)-benzyl]-(3,4-dichloro-phenyl)-amino]-oxazol-5-yl}-benzoic acid methyl ester
4-{2-[[3-Bromo-4-(difluoro-phosphono-methyl)-benzyl]-(3,4-dichloro-phenyl)-amino]-oxazol-5-yl}-benzoic acid
4-(2-{[3-Bromo-4-(difluoro-phosphono-methyl)-benzyl]-phenyl-amino}-oxazol-5-yl)-benzoic acid methyl ester
4-(2-{[3-Bromo-4-(difluoro-phosphono-methyl)-benzyl]-phenyl-amino}-oxazol-5-yl)-benzoic acid
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid
4-(4-{2-[ {3-Bromo-4-[(diethoxy-phosphoryl)-difluoro-methyl]-benzyl}-(3,4-dichloro-phenyl)-amino]-oxazol-5-yl}-phenoxy)-benzoic acid methyl ester
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid diethyl ester
[2-Bromo-4-({(3,4-dichloro-phenyl)-[5-(4-methanesulfonyl-phenyl)-oxazol-2-yl]-amino}-methyl)-phenoxy]-acetic acid
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monoethyl ester
3-[2-Bromo-4-({(3,4-dichloro-phenyl)-[5-(4-methanesulfonyl-phenyl)-oxazol-2-yl]-amino}-methyl)-phenyl]-acrylic acid
4-(4-{2-[[3-Bromo-4-(difluoro-phosphono-methyl)-benzyl]-(3,4-dichloro-phenyl)-amino]-oxazol-5-yl}-phenoxy)-benzoic acid methyl ester
[4-({(3,4-Dichloro-phenyl)-[5-(4-methanesulfonyl-phenyl)-oxazol-2-yl]-amino}-methyl)-phenyl]-difluoro-acetic acid
2,2-Dimethyl-propionic acid {[2-bromo-4-({(3,4-dichloro-phenyl)-[5-(4-methanesulfonyl-phenyl)-oxazol-2-yl]-amino}-methyl)-phenyl]-difluoro-methyl}-hydroxy-phosphinoyloxymethyl ester
4-(4-{2-[[3-Bromo-4-(difluoro-phosphono-methyl)-benzyl]-(3,4-dichloro-phenyl)-amino]-oxazol-5-yl}-phenoxy)-benzoic acid
(3-{2-[[3-Bromo-4-(difluoro-phosphono-methyl)-benzyl]-(3,4-dichloro-phenyl)-amino]-oxazol-5-yl}-phenoxy)-phenyl-acetic acid methyl ester
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monomethyl ester
2-(3-{2-[[3-Bromo-4-(difluoro-phosphono-methyl)-benzyl]-(3,4-dichloro-phenyl)-amino]-oxazol-5-yl}-phenoxy)-benzoic acid methyl ester
4′-{2-[[3-Bromo-4-(difluoro-phosphono-methyl)-benzyl]-(3,4-dichloro-phenyl)-amino]-oxazol-5-yl}-biphenyl-2-carboxylic acid methyl ester
(3-{2-[[3-Bromo-4-(difluoro-phosphono-methyl)-benzyl]-(3,4-dichloro-phenyl)-amino]-oxazol-5-yl}-phenoxy)-phenyl-acetic acid
4′-{2-[[3-Bromo-4-(difluoro-phosphono-methyl)-benzyl]-(3,4-dichloro-phenyl)-amino]-oxazol-5-yl}-biphenyl-2-carboxylic acid
2-(3-{2-[[3-Bromo-4-(difluoro-phosphono-methyl)-benzyl]-(3,4-dichloro-phenyl)-amino]-oxazol-5-yl}-phenoxy)-benzoic acid
3′-{2-[[3-Bromo-4-(difluoro-phosphono-methyl)-benzyl]-(3,4-dichloro-phenyl)-amino]-oxazol-5-yl}-biphenyl-2-carboxylic acid methyl ester
3′-{2-[[3-Bromo-4-(difluoro-phosphono-methyl)-benzyl]-(3,4-dichloro-phenyl)-amino]-oxazol-5-yl}-biphenyl-2-carboxylic acid
4-({[4-(Difluoro-phosphono-methyl)-benzyl]-[5-(3-phenoxy-phenyl)-[1,3,4]oxadiazol-2-yl]-amino}-methyl)-benzoic acid methyl ester
4-({[4-(Difluoro-phosphono-methyl)-benzyl]-[5-(3-phenoxy-phenyl)-[1,3 ,4]oxadiazol-2-yl]-amino}-methyl)-benzoic acid
3-(3-{5-[[3- benzyl]-(3,4-dichloro-phenyl)-amino]-[1,3,4]oxadiazol-2-yl}-phenoxy)-benzoic acid
3-[3-(5-{(3,4-Dichloro-phenyl)-[4-(difluoro-phosphono-methyl)-benzyl]-amino}-[1,3,4]oxadiazol-2-yl)-phenoxy]-benzoic acid methyl ester
3-[3-(5-{(3,4-Dichloro-phenyl)-[4-(difluoro-phosphono-methyl)-benzyl]-amino}-[1,3,4]oxadiazol-2-yl)-phenoxy]-benzoic acid
3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid methyl ester
3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid
[(2-Bromo-4-{[(3,4-dichloro-phenyl)-(5-phenyl-oxazol-2-yl)-amino]-methyl}-phenyl)-difluoro-methyl]-phosphonic acid
4-{2-[[3-Bromo-4-(difluoro-phosphono-methyl)-benzyl]-(3,4-dichloro-phenyl)-amino]-oxazol-5-yl}-benzoic acid methyl ester
4-{2-[[3-Bromo-4-(difluoro-phosphono-methyl)-benzyl]-(3,4-dichloro-phenyl)-amino]-oxazol-5-yl}-benzoic acid
4-(2-{[3-Bromo-4-(difluoro-phosphono-methyl)-benzyl]-phenyl-amino}-oxazol-5-yl)-benzoic acid methyl ester
4-(2-{[3-Bromo-4-(difluoro-phosphono-methyl)-benzyl]-phenyl-amino}-oxazol-5-yl)-benzoic acid
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid
4-(4-{2-[ {3-Bromo-4-[(diethoxy-phosphoryl)-difluoro-methyl]-benzyl}-(3,4-dichloro-phenyl)-amino]-oxazol-5-yl}-phenoxy)-benzoic acid methyl ester
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid diethyl ester
[2-Bromo-4-({(3,4-dichloro-phenyl)-[5-(4-methanesulfonyl-phenyl)-oxazol-2-yl]-amino}-methyl)-phenoxy]-acetic acid
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monoethyl ester
3-[2-Bromo-4-({(3,4-dichloro-phenyl)-[5-(4-methanesulfonyl-phenyl)-oxazol-2-yl]-amino}-methyl)-phenyl]-acrylic acid
4-(4-{2-[[3-Bromo-4-(difluoro-phosphono-methyl)-benzyl]-(3,4-dichloro-phenyl)-amino]-oxazol-5-yl}-phenoxy)-benzoic acid methyl ester
[4-({(3,4-Dichloro-phenyl)-[5-(4-methanesulfonyl-phenyl)-oxazol-2-yl]-amino}-methyl)-phenyl]-difluoro-acetic acid
2,2-Dimethyl-propionic acid {[2-bromo-4-({(3,4-dichloro-phenyl)-[5-(4-methanesulfonyl-phenyl)-oxazol-2-yl]-amino}-methyl)-phenyl]-difluoro-methyl}-hydroxy-phosphinoyloxymethyl ester
4-(4-{2-[[3-Bromo-4-(difluoro-phosphono-methyl)-benzyl]-(3,4-dichloro-phenyl)-amino]-oxazol-5-yl}-phenoxy)-benzoic acid
(3-{2-[[3-Bromo-4-(difluoro-phosphono-methyl)-benzyl]-(3,4-dichloro-phenyl)-amino]-oxazol-5-yl}-phenoxy)-phenyl-acetic acid methyl ester
{[2-Bromo-4-({(3,4-dichloro-phenyl)-[5-(4-methanesulfonyl-phenyl)-oxazol-2-yl]-amino}-methyl)-phenyl]-difluoro-methyl}-phosphonic acid monomethyl ester
2-(3-{2-[[3-Bromo-4-(difluoro-phosphono-methyl)-benzyl]-(3,4-dichloro-phenyl)-amino]-oxazol-5-yl}-phenoxy)-benzoic acid methyl ester
4′-{2-[[3-Bromo-4-(difluoro-phosphono-methyl)-benzyl]-(3,4-dichloro-phenyl)-amino]-oxazol-5-yl}-biphenyl-2-carboxylic acid methyl ester
(3-{2-[[3-Bromo-4-(difluoro-phosphono-methyl)-benzyl]-(3,4-dichloro-phenyl)-amino]-oxazol-5-yl}-phenoxy)-phenyl-acetic acid
4′-{2-[[3-Bromo-4-(difluoro-phosphono-methyl)-benzyl]-(3,4-dichloro-phenyl)-amino]-oxazol-5-yl}-biphenyl-2-carboxylic acid
2-(3-{2-[[3-Bromo-4-(difluoro-phosphono-methyl)-benzyl]-(3,4-dichloro-phenyl)-amino]-oxazol-5-yl}-phenoxy)-benzoic acid
3′-{2-[[3-Bromo-4-(difluoro-phosphono-methyl)-benzyl]-(3,4-dichloro-phenyl)-amino]-oxazol-5-yl}-biphenyl-2-carboxylic acid methyl ester
3′-{2-[[3-Bromo-4-(difluoro-phosphono-methyl)-benzyl]-(3,4-dichloro-phenyl)-amino]-oxazol-5-yl}-biphenyl-2-carboxylic acid
4-({[4-(Difluoro-phosphono-methyl)-benzyl]-[5-(3-phenoxy-phenyl)-[ I ,3,4]oxadiazol-2-yl]-amino}-methyl)-benzoic acid methyl ester
4-({[4-(Difluoro-phosphono-methyl)-benzyl]-[5-(3-phenoxy-phenyl)-[1,3 ,4]oxadiazol-2-yl]-amino}-methyl)-benzoic acid
3-(3-{5-[[3- benzyl]-(3,4-dichloro-phenyl)-amino]-[1,3,4]oxadiazol-2-yl}-phenoxy)-benzoic acid
3-[3-(5-{(3,4-Dichloro-phenyl)-[4-(difluoro-phosphono-methyl)-benzyl]-amino}-[1,3,4]oxadiazol-2-yl)-phenoxy]-benzoic acid methyl ester
3-[3-(5-{(3,4-Dichloro-phenyl)-[4-(difluoro-phosphono-methyl)-benzyl]-amino}-[1,3,4]oxadiazol-2-yl)-phenoxy]-benzoic acid
3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid methyl ester; and
3-(3-{5-[[3-Bromo-4-(difluoro-phosphono-methyl)-benzyl]-(3,4-dichloro-phenyl)-amino]-[1,3,4]oxadiazol-2-yl}-phenoxy)-benzoic acid.

16. A pharmaceutical composition comprising the compound of claim I and a pharmaceutically acceptable carrier.

17. The pharmaceutical composition of claim 16 that is formulated for single dosage administration.

18. A method for treating or ameliorating one or more symptoms of diabetes in a mammal, comprising administering a compound of claim 1 to said mammal.

19. A method for treating obesity in a mammal, comprising administering a compound of claim 1 to said mammal.

20. A method for improving insulin sensitivity or leptin sensitivity in a mammal, comprising administering a compound of claim 1 to said mammal.

21. The method of claim 18, further comprising administering insulin, an insulin sensitizer, and/or leptin to said mammal.

22. A method for facilitating the loss of body weight in a mammal, comprising administering a compound of claim 1 to said mammal.

23. A method for treating or ameliorating one or more symptoms of a neurodegenerative disease in a mammal, comprising administering a compound of claim 1 to said mammal.

24. A method for treating or ameliorating one or more symptoms of cancer in a mammal, comprising administering a compound of claim 1 to said mammal.

25. A prodrug of a compound of claim 1, wherein the prodrug has the formula ArCF₂P(O)(OH)(OCH(H/Me)OC(═O)OiPr, ArCF₂P(O)[(OCH(H/Me)OC(═O)OiPr]₂, ArCF₂P(O)(OH)(OCH(H/Me)OC(═O)tBu, or ArCF₂P(O)[(OCH(H/Me)OC(═O)tBu]₂.

26. A prodrug of a compound of claim 1 that is a mono- or bis-amidate prodrug, a mono- or di-lipid ester prodrug, a mono- or di-alpha-acyloxyalkyl ester or amide prodrug, a cytochrome P450 3 A activated prodrug, a cyclic diester prodrug, a cyclic monoester monoamide prodrug, a cyclic diamide prodrug, or a carbohydrate prodrug.

27. A prodrug of a compound of claim 1, wherein the prodrug has the formula ROCH₂CHR′CH₂O—P(O)(OH)CF₂Ar or (ROCH₂CHR′CH₂O)₂—P(O)CF₂Ar, where R is C₁₄₋₂₀-n-alkyl and R′ is H, OH or OMe.
